SEQUENCE LISTING (90 sequences)

SEQ ID NO: 1 - protein, 4 aa, Artificial sequence
  Synthesized
  MISC_FEATURE (1)..(1): Xaa is Glu, Asp, Asn, or Gln
  MISC_FEATURE (2)..(2): Xaa is Ser or Thr
  MISC_FEATURE (3)..(3): Xaa is Asp, Glu, Gln, or Asn
  MISC_FEATURE (4)..(4): Xaa is Val or Leu
XXXX

SEQ ID NO: 2 - protein, 4 aa, Artificial sequence
  Synthesized
  MISC_FEATURE (1)..(1): Xaa is Glu, Gln, Ala, or an analog thereof
  MISC_FEATURE (2)..(2): Xaa is Thr or Ser
  MISC_FEATURE (3)..(3): Xaa is Ala, Gln, Asp, Asn, N-Me-Ala, N-Me-Gln, N-Me-Asp, N-Me-Asn, or an analog thereof
XXXV

SEQ ID NO: 3 - protein, 20 aa, Artificial sequence
  Synthesized
YGRKKRRQRRRKLSSIESDV

SEQ ID NO: 4 - protein, 15 aa, Artificial sequence
  Synthesized
RRQRRRKLSSIESDV

SEQ ID NO: 5 - protein, 14 aa, Artificial sequence
  Synthesized
RQRRRKLSSIESDV

SEQ ID NO: 6 - protein, 13 aa, Artificial sequence
  Synthesized
QRRRKLSSIESDV

SEQ ID NO: 7 - protein, 11 aa, Artificial sequence
  Synthesized
RRKLSSIESDV

SEQ ID NO: 8 - protein, 10 aa, Artificial sequence
  Synthesized
RKLSSIESDV

SEQ ID NO: 9 - protein, 9 aa, Artificial sequence
  Synthesized
KLSSIESDV

SEQ ID NO: 10 - protein, 8 aa, Artificial sequence
  Synthesized
LSSIESDV

SEQ ID NO: 11 - protein, 9 aa, Artificial sequence
  Synthesized
RKKRRQRRR

SEQ ID NO: 12 - protein, 4 aa, Artificial sequence
  Synthesized
ESDV

SEQ ID NO: 13 - protein, 4 aa, Artificial sequence
  Synthesized
ESEV

SEQ ID NO: 14 - protein, 4 aa, Artificial sequence
  Synthesized
ETDV

SEQ ID NO: 15 - protein, 4 aa, Artificial sequence
  Synthesized
ETAV

SEQ ID NO: 16 - protein, 4 aa, Artificial sequence
  Synthesized
ETEV

SEQ ID NO: 17 - protein, 4 aa, Artificial sequence
  Synthesized
DTDV

SEQ ID NO: 18 - protein, 4 aa, Artificial sequence
  Synthesized
DTEV

SEQ ID NO: 19 - protein, 5 aa, Artificial sequence
  Synthesized
  MISC_FEATURE (2)..(2): Xaa is Glu, Asp, Asn, or Gln
  MISC_FEATURE (3)..(3): Xaa is Ser or Thr
  MISC_FEATURE (4)..(4): Xaa is Asp, Glu, Gln, or Asn
  MISC_FEATURE (5)..(5): Xaa is Val or Leu
IXXXX

SEQ ID NO: 20 - protein, 5 aa, Artificial sequence
  Synthesized
IESDV

SEQ ID NO: 21 - protein, 5 aa, Artificial sequence
  Synthesized
IESEV

SEQ ID NO: 22 - protein, 5 aa, Artificial sequence
  Synthesized
IETDV

SEQ ID NO: 23 - protein, 5 aa, Artificial sequence
  Synthesized
IETAV

SEQ ID NO: 24 - protein, 5 aa, Artificial sequence
  Synthesized
IETEV

SEQ ID NO: 25 - protein, 5 aa, Artificial sequence
  Synthesized
IDTDV

SEQ ID NO: 26 - protein, 5 aa, Artificial sequence
  Synthesized
IDTEV

SEQ ID NO: 27 - protein, 4 aa, Artificial sequence
  Synthesized
  MISC_FEATURE (1)..(1): Xaa is Glu, Asp, Gln, Ala, or an analog thereof
  MISC_FEATURE (2)..(2): Xaa is Thr or Ser
  MISC_FEATURE (3)..(3): Xaa is Ala, Gln, Asp, Glu, Asn, N-Me-Ala, N-Me-Gln, N-Me-Asp, N-Me-Glu, N-Me-Asn, or an analog thereof
XXXV

SEQ ID NO: 28 - protein, 5 aa, Artificial sequence
  Synthesized
  MISC_FEATURE (2)..(2): Xaa is Glu, Gln, Ala, or an analog thereof
  MISC_FEATURE (3)..(3): Xaa is Thr or Ser
  MISC_FEATURE (4)..(4): Xaa is Ala, Gln, Asp, Asn, N-Me-Ala, N-Me-Gln, N-Me-Asp, N-Me-Asn, or an analog thereof
IXXXV

SEQ ID NO: 29 - protein, 5 aa, Artificial sequence
  Synthesized
  MISC_FEATURE (2)..(2): Xaa is Glu, Gln, Ala, or Asp, or an analog
  MISC_FEATURE (3)..(3): Xaa is Thr or Ser
  MISC_FEATURE (4)..(4): Xaa is Ala, Gln, Asp, Glu, Asn, N-Me-Ala, N-Me-Gln, N-Me-Asp, N-Me-Glu, N-Me-Asn, or an analog thereof
IXXXV

SEQ ID NO: 30 - protein, 9 aa, Artificial sequence
  Synthesized
KLSSIETDV

SEQ ID NO: 31 - protein, 14 aa, Artificial sequence
  Synthesized
RLSGMNEVLSFRWL

SEQ ID NO: 32 - protein, 10 aa, Artificial sequence
  Synthesized
GRKKRRQRRR

SEQ ID NO: 33 - protein, 5 aa, Artificial sequence
  Synthesized
GSSSS

SEQ ID NO: 34 - protein, 5 aa, Artificial sequence
  Synthesized
TGEKP

SEQ ID NO: 35 - protein, 8 aa, Artificial sequence
  Synthesized
GGRRGGGS

SEQ ID NO: 36 - protein, 9 aa, Artificial sequence
  Synthesized
LRQRDGERP

SEQ ID NO: 37 - protein, 13 aa, Artificial sequence
  Synthesized
YGRKKRRQRRRPQ

SEQ ID NO: 38 - protein, 12 aa, Artificial sequence
  Synthesized
GRKKRRQRRRPQ

SEQ ID NO: 39 - protein, 11 aa, Artificial sequence
  Synthesized
GRKKRRQRRRP

SEQ ID NO: 40 - protein, 11 aa, Artificial sequence
  Synthesized
  MISC_FEATURE (1)..(1): Xaa is an amino acid other than Tyr
XGRKKRRQRRR

SEQ ID NO: 41 - protein, 11 aa, Artificial sequence
  Synthesized
FGRKKRRQRRR

SEQ ID NO: 42 - protein, 10 aa, Artificial sequence
  Synthesized
RRRQRRKKRG

SEQ ID NO: 43 - protein, 11 aa, Artificial sequence
  Synthesized
RRRQRRKKRGY

SEQ ID NO: 44 - protein, 10 aa, Artificial sequence
  Synthesized
GKKKKKQKKK

SEQ ID NO: 45 - protein, 10 aa, Artificial sequence
  Synthesized
GAKKRRQRRR

SEQ ID NO: 46 - protein, 9 aa, Artificial sequence
  Synthesized
AKKRRQRRR

SEQ ID NO: 47 - protein, 10 aa, Artificial sequence
  Synthesized
GRKARRQRRR

SEQ ID NO: 48 - protein, 9 aa, Artificial sequence
  Synthesized
RKARRQRRR

SEQ ID NO: 49 - protein, 10 aa, Artificial sequence
  Synthesized
GRKKARQRRR

SEQ ID NO: 50 - protein, 9 aa, Artificial sequence
  Synthesized
RKKARQRRR

SEQ ID NO: 51 - protein, 10 aa, Artificial sequence
  Synthesized
GRKKRRQARR

SEQ ID NO: 52 - protein, 9 aa, Artificial sequence
  Synthesized
RKKRRQARR

SEQ ID NO: 53 - protein, 10 aa, Artificial sequence
  Synthesized
GRKKRRQRAR

SEQ ID NO: 54 - protein, 9 aa, Artificial sequence
  Synthesized
RKKRRQRAR

SEQ ID NO: 55 - protein, 11 aa, Artificial sequence
  Synthesized
RRPRRPRRPRR

SEQ ID NO: 56 - protein, 11 aa, Artificial sequence
  Synthesized
RRARRARRARR

SEQ ID NO: 57 - protein, 10 aa, Artificial sequence
  Synthesized
RRRARRRARR

SEQ ID NO: 58 - protein, 10 aa, Artificial sequence
  Synthesized
RRRPRRRPRR

SEQ ID NO: 59 - protein, 8 aa, Artificial sequence
  Synthesized
RRPRRPRR

SEQ ID NO: 60 - protein, 8 aa, Artificial sequence
  Synthesized
RRARRARR

SEQ ID NO: 61 - protein, 20 aa, Artificial sequence
  Synthesized
YGRKKRRQRRRKLSSIETDV

SEQ ID NO: 62 - protein, 20 aa, Artificial sequence
  Synthesized
  MISC_FEATURE (1)..(15): D-amino acids
YGRKKRRQRRRKLSSIESDV

SEQ ID NO: 63 - protein, 20 aa, Artificial sequence
  Synthesized
  MISC_FEATURE (1)..(16): D-amino acids
YGRKKRRQRRRKLSSIESDV

SEQ ID NO: 64 - protein, 20 aa, Artificial sequence
  Synthesized
  MISC_FEATURE (1)..(14): D-amino acids
YGRKKRRQRRRKLSSIESDV

SEQ ID NO: 65 - protein, 20 aa, Artificial sequence
  Synthesized
  MISC_FEATURE (1)..(13): D-amino acids
YGRKKRRQRRRKLSSIESDV

SEQ ID NO: 66 - protein, 19 aa, Artificial sequence
  Synthesized
  MISC_FEATURE (1)..(14): D-amino acids
YGRKKRRQRRRKSSIESDV

SEQ ID NO: 67 - protein, 18 aa, Artificial sequence
  Synthesized
  MISC_FEATURE (1)..(13): D-amino acids
YGRKKRRQRRRKSIESDV

SEQ ID NO: 68 - protein, 17 aa, Artificial sequence
  Synthesized
  MISC_FEATURE (1)..(12): D-amino acids
YGRKKRRQRRRKIESDV

SEQ ID NO: 69 - protein, 20 aa, Artificial sequence
  Synthesized
  MISC_FEATURE (1)..(15): D-amino acids
YGRKKRRQRRRKLSSIETDV

SEQ ID NO: 70 - protein, 20 aa, Artificial sequence
  Synthesized
  MISC_FEATURE (1)..(16): D-amino acids
YGRKKRRQRRRKLSSIETDV

SEQ ID NO: 71 - protein, 20 aa, Artificial sequence
  Synthesized
  MISC_FEATURE (1)..(14): D-amino acids
YGRKKRRQRRRKLSSIETDV

SEQ ID NO: 72 - protein, 20 aa, Artificial sequence
  Synthesized
  MISC_FEATURE (1)..(13): D-amino acids
YGRKKRRQRRRKLSSIETDV

SEQ ID NO: 73 - protein, 19 aa, Artificial sequence
  Synthesized
  MISC_FEATURE (1)..(14): D-amino acids
YGRKKRRQRRRKSSIETDV

SEQ ID NO: 74 - protein, 18 aa, Artificial sequence
  Synthesized
  MISC_FEATURE (1)..(13): D-amino acids
YGRKKRRQRRRKSIETDV

SEQ ID NO: 75 - protein, 17 aa, Artificial sequence
  Synthesized
  MISC_FEATURE (1)..(12): D-amino acids
YGRKKRRQRRRKIETDV

SEQ ID NO: 76 - protein, 16 aa, Artificial sequence
  Synthesized
  MISC_FEATURE (1)..(11): D-amino acids
YGRKKRRQRRRIESDV

SEQ ID NO: 77 - protein, 16 aa, Artificial sequence
  Synthesized
  MISC_FEATURE (1)..(11): D-amino acids
YGRKKRRQRRRIETDV

SEQ ID NO: 78 - protein, 20 aa, Artificial sequence
  Synthesized
  MISC_FEATURE (1)..(15): D-amino acids
YGRKKRRQRRRKLSSIESDV

SEQ ID NO: 79 - protein, 20 aa, Artificial sequence
  Synthesized
  MISC_FEATURE (1)..(20): D-amino acids
VDSEISSLKRRRQRRKKRGY

SEQ ID NO: 80 - protein, 9 aa, Artificial sequence
  Synthesized
  MISC_FEATURE (1)..(2): D-amino acids
KLSSIESDV

SEQ ID NO: 81 - protein, 8 aa, Artificial sequence
  Synthesized
  MISC_FEATURE (1)..(1): D-amino acid
LSSIESDV

SEQ ID NO: 82 - protein, 7 aa, Artificial sequence
  Synthesized
SSIESDV

SEQ ID NO: 83 - protein, 6 aa, Artificial sequence
  Synthesized
SIESDV

SEQ ID NO: 84 - protein, 9 aa, Artificial sequence
  Synthesized
  MISC_FEATURE (1)..(2): D-amino acids
KLSSIETDV

SEQ ID NO: 85 - protein, 8 aa, Artificial sequence
  Synthesized
  MISC_FEATURE (1)..(1): D-amino acid
LSSIETDV

SEQ ID NO: 86 - protein, 8 aa, Artificial sequence
  Synthesized
LSSIETDV

SEQ ID NO: 87 - protein, 7 aa, Artificial sequence
  Synthesized
SSIETDV

SEQ ID NO: 88 - protein, 6 aa, Artificial sequence
  Synthesized
SIETDV

SEQ ID NO: 89 - protein, 20 aa, Artificial sequence
  Synthesized
  MISC_FEATURE (1)..(11): D-amino acids
YGRKKRRQRRRKLSSIESDV

SEQ ID NO: 90 - protein, 9 aa, Artificial sequence
  Synthesized
KLSSIEADA

(19) United States
(12) Patent Application Publication
(10) Pub. No.: US 2023/0139826 A1
(43) Pub. Date: May 4, 2023

(54) COMBINATION TREATMENT OF STROKE WITH PLASMIN-CLEAVABLE PSD-95 INHIBITOR AND REPERFUSION

(71) Applicant: NONO INC., Toronto (CA)

(72) Inventors: Michael TYMIANSKI, Brockville (CA); Jonathan David GARMAN, Thornhill (CA)

(21) Appl. No.: 17/800,523

(22) PCT Filed: Feb. 19, 2021
(86) PCT No.: PCT/IB2021/051405

§ 371 Date: Aug. 17, 2022

Related U.S. Application Data
(60) Provisional application No. 62/978,759, filed on Feb. 19, 2020.
(60) Provisional application No. 62/978,792, filed on Feb. 19, 2020.

(51) Int. Cl. A61K 38/10; A61P 9/10

(52) U.S. Cl. CPC A61K 38/10; A61P 9/10

(57) ABSTRACT

The peptide inhibitor of PSD-95, Tat-NR2B9c, is cleaved by the serum protease, plasmin, inducible by thrombolytic agents. Conversely, Tat-NR2B9c has no detrimental effect on the activity of a thrombolytic agent. Inactivation of Tat-NR2B9c by thrombolytic agents can be reduced or avoided by several approaches including spacing the administration of the respective agents to avoid substantial overlap in plasma residence between Tat-NR2B9c and plasmin, using mechanical instead of thrombolytic reperfusion or using active agent that inhibits PSD-95 not subject to cleavage by plasmin, e.g., D-amino acid variants of Tat-NR2B9c.

### CROSS-REFERENCE TO RELATED APPLICATIONS

This application claims priority from U.S. 62/978,759 and U.S. 62/978,792, each filed Feb. 19, 2020, each incorporated by reference in its entirety for all purposes.

### SEQUENCE LISTING

This application includes sequences disclosed in a txt filed named 552735SEQLST.TXT, of 22,109 bytes, created Feb. 17, 2021, which is incorporated by reference.

### BACKGROUND

Tat-NR2B9c (also known as NA-1 or nerinetide) is an agent that inhibits PSD-95, thus disrupting binding to N-methyl-D-aspartate receptors (NMDARs) and neuronal nitric oxide synthases (nNOS) and reducing excitoxicity induced by cerebral ischemia. Treatment reduces infarction size and functional deficits in models of cerebral injury and neurodegenerative diseases. Tat-NR2B9c has undergone a successful phase II trial (see WO 2010144721 and Aarts et al., Science 298, 846-850 (2002), Hill et al., Lancet Neurol. 11:942-950 (2012)) and a successful Phase 3 trial (Hill et al, Lancet 395:878-887 (2020)).

### SUMMARY OF THE CLAIMED INVENTION

The invention provides a method of treating a population of subjects having or at risk of ischemia comprising administering to the subjects an active agent that inhibits PSD-95, cleavable by plasmin, and reperfusion. The population of subjects includes subjects administered the active agent that inhibits PSD-95 and mechanical reperfusion or a vasodilator agent or a hypertensive agent to effect reperfusion; and/or subjects administered the active agent that inhibits PSD-95 and a thrombolytic agent to effect reperfusion, wherein the active agent that inhibits PSD-95 is administered at least 10 minutes before the thrombolytic agent, and the population of subjects lacks subjects in which a thrombolytic agent is administered less than 3 hours before or less than 10 minutes after administering the active agent that inhibits PSD-95.

Optionally, the subjects have ischemic stroke. Optionally, the population lacks subjects in which the thrombolytic agent is administered less than four hours before the active agent that inhibits PSD-95 or less than 10 minutes after the active agent that inhibits PSD-95. Optionally, the population lacks subjects in which the thrombolytic agent is administered less than eight hours before the active agent that inhibits PSD-95 and less than 10 minutes after administering the active agent that inhibits PSD-95. Optionally, the population lacks subjects in which the thrombolytic agent is administered before the active agent that inhibits PSD-95 or less than ten minutes after administering the active agent that inhibits PSD-95. Optionally, the population lacks subjects in which the thrombolytic agent is administered before the PSD-95 inhibitor or less than 20 minutes after administering the active agent that inhibits PSD-95. Optionally, the population lacks subjects in which the thrombolytic agent is administered before the active agent that inhibits PSD-95 or less than 30 minutes after administering the active agent that inhibits PSD-95. Optionally, the population lacks subjects in which the thrombolytic agent is administered before the active agent that inhibits PSD-95 or less than 60 minutes after administering the active agent that inhibits PSD-95. Optionally, the population of subjects includes subjects administered the active agent that inhibits PSD-95 and mechanical reperfusion without receiving a thrombolytic agent.

Optionally, the population of treated subjects consists of: (a) subjects administered the active agent that inhibits PSD-95 and mechanical reperfusion, a vasodilator agent or a hypertensive agent without a thrombolytic agent; and (b) subjects administered the active agent that inhibits PSD-95 and a thrombolytic agent, wherein the thrombolytic agent is administered at least 10, 20, 30, 60, or 120 minutes after the active agent that inhibits PSD-95. Optionally, at least some of the subjects according to item (b) also are administered mechanical reperfusion. Optionally, the population includes subjects in which the thrombolytic agent is administered more than 3 or 4.5 hours after onset of stroke when the subjects were determined to be eligible for treatment with the thrombolytic agent less than 3 hours after onset of stroke. Optionally, the population includes at least 100 subjects. Optionally, the population includes subjects in which the active agent that inhibits PSD-95 is administered over a ten minute period and the thrombolytic agents is administered at least 30 minutes from the start of administering the active agent. Optionally, the active agent is a peptide of all L-amino acids. Optionally, the active agent is nerinetide.

The invention further provides a method of treating a population of subjects receiving endovascular thrombectomy for ischemic stroke comprising administering both an active agent that inhibits PSD-95 cleavable by plasmin and a thrombolytic agent to some of the subjects, wherein the active agent that inhibits PSD-95 is administered at least 10, 20, 30, 60 or 120 minutes before the thrombolytic agent, and administering the active agent that inhibits PSD-95 or the thrombolytic agent but not both to other subjects. Optionally, the subjects receiving the active agent that inhibits PSD-95 and thrombolytic agent do so before the subjects receive endovascular thrombectomy. Optionally, the subjects receiving the active agent that inhibits PSD-95 or thrombolytic agent but not both do before the subjects receive endovascular thrombectomy. Optionally, in the subjects receiving both the active agent that inhibits PSD-95 and thrombolytic agent, the active agent that inhibits PSD-95 is administered at least 10 minutes before the thrombolytic agent, and the active agent that inhibits PSD-95 or the thrombolytic agent but not both is administered to the other subjects.

The invention further provides a method of treating a population of subjects having or at risk of ischemia, comprising administering to the subjects an active agent that inhibits PSD-95, and a thrombolytic agent, wherein the population of subjects includes: subjects administered a first active agent that inhibits PSD-95 cleavable by plasmin and a thrombolytic agent, wherein the first active agent that inhibits PSD-95 is administered at an interval selected from at least 10, 20, 30, 60 or 120 minutes before the thrombolytic agent; and subjects administered a second active agent that inhibits PSD-95 resistant to cleavage by plasmin and a thrombolytic agent, wherein the thrombolytic agent is administered before or within the interval after the active agent that inhibits PSD-95.

The invention further provides a method of treating a subject suspected of having ischemic stroke, comprising: determining eligibility of the subject for treatment with a thrombolytic agent; administering an active agent that inhibits PSD-95, cleavable by plasmin; and at least 10, 20, 30, 60 or 120 minutes thereafter administering the thrombolytic agent. Optionally, the active agent that inhibits PSD-95 is administered over a ten minute period and the thrombolytic agent is administered at least 20 minutes from the start of administering the active agent. Optionally, the active agent is a peptide of all L-amino acids, optionally nerinetide. Optionally, the imaging determines presence of ischemic stroke and absence of cerebral hemorrhage. Optionally, eligibility is determined less than 3 hours after onset of stroke and the thrombolytic agent is administered more than 3 hours after onset of ischemic stroke. Optionally, eligibility is determine less than 4.5 hours after onset of ischemic stroke and the thrombolytic agent is administered more than 4.5 hours after onset of ischemic stroke. Optionally, eligibility is determined less than 3 hours after onset of ischemic stroke and the thrombolytic agent is administered more than 4.5 hours after onset of ischemic stroke.

In any of the above methods, the active agent that inhibits PSD-95 can comprise a peptide comprising [E/D/N/Q]-[S/T]-[D/E/Q/N]-[V/L] (SEQ ID NO:1) at the C-terminus or X₁-[T/S]-X₂-V (SEQ ID NO:2) at the C-terminus, wherein [T/S]are alternative amino acids, X₁ is selected from among E, Q, and A, or an analogue thereof, X₂ is selected from among A, Q, D, N, N-Me-A, N-Me-Q, N-Me-D, and N-Me-N or an analog thereof, and an internalized peptide linked to the N-terminus of the peptide. Optionally, the active agent that inhibits PSD-95 linked to the internalization peptide is Tat-NR2B9c (nerinetide). Optionally, the thrombolytic agent is tPA.

The invention further provides a method of treating a subject who has had a stroke with a plasmin-sensitive active agent that inhibits PSD-95, i.e., cleavable by plasmin, whereby the plasmin-sensitive inhibitor is administered at least 10 minutes before a thrombolytic agent, or administered at least 2, 3, 4 or more hours after administration of a thrombolytic agent, or administered without a thrombolytic agent. Optionally, the active agent that inhibits PSD-95 is administered over a ten minute period and the thrombolytic agent is administered at least 20 minutes from the start of administering the active agent. Optionally, the active agent is a peptide of all L-amino acids. Optionally, the active agent is nerinetide.

The invention further provides a method of minimizing degradation of a plasmin-sensitive active agent that inhibits PSD-95 (i.e., cleavable by plasmin) by a thrombolytic agent, comprising: administering the active agent that inhibits PSD-95 at least 10 minutes before the thrombolytic agent, or administering the active agent that inhibits PSD-95 at least 2, 3, 4 or more hours after administration of the thrombolytic agent, or administering the active agent that inhibits PSD-95 without the thrombolytic agent, or administering the active agent that inhibits PSD-95 by intranasal or intrathecal administration. Optionally, the active agent that inhibits PSD-95 is administered over a ten minute period and the thrombolytic agent is administered at least 20 minutes from the start of administering the active agent. Optionally, the active agent is a peptide of all L-amino acids. Optionally, the active agent is nerinetide.

The invention further provides a method of treating ischemic stroke, comprising administering to a subject having ischemic stroke and active agent that inhibits PSD-95, cleavable by plasmin, and 20-40 minutes after initiating administration of the active agent administering a thrombolytic agent. Optionally, the active agent that inhibits PSD-95 is inhibited over a period of ten minutes and the thrombolytic agent is administered 20-30 minutes after initiating administration of the active agent.

### BRIEF DESCRIPTIONS OF THE FIGURES

FIG. 1: Plasma levels of nerinetide with and without alteplase administration.

FIG. 2A: Horizontal stacked bar graphs showing the primary outcome distribution on the modified Rankin Scale by nerinetide treatment group. Bars are labelled with proportions.

FIG. 2B: Horizontal stacked bar graphs showing the primary outcome distribution on the modified Rankin Scale by nerinetide treatment group according to usual care alteplase treatment. Bars are labelled with proportions.

FIG. 3: Forest plots of nerinetide treatment effect in pre-specified subgroups. Comparisons are unadjusted for multiplicity. Effect sizes, adjusted for the same variables as the primary analysis (alteplase, endovascular approach, age, sex, NIHSS score, ASPECTS, occlusion location, site), are shown by randomization strata and then according to additional pre-specified sub-groups. Two pre-specified sub-groups are not included in this plot: (1) onset to treatment time <=4 h or >4 h because this was redundant with the similar grouping using a 6-hour time threshold; (2) weight >105-120 kg vs. 40-105 kg because so few patients fell into the high weight category that modelling became unstable. There is significant overlap between onset-to-treatment time >6 hours and the no alteplase stratum because in usual care patients in later time windows are not treated with intravenous alteplase.

FIGS. 4A-E. Nerinetide is cleaved by plasmin. (A) LC/MS spectrum of nerinetide after incubation with plasmin in PBS. 10 uL aliquots of nerinetide (18 mg/mL) and plasmin (1 mg/mL) were incubated in 500 uL tubes of phosphate-buffered saline at 37 C for 5 min and the reaction stopped by cooling to −80 C until tested. The various peaks correspond to the indicated fragments. Insert: Predicted trypsin cleavage sites and actual cleavage sites. (B, C) In-vitro effect of rt-PA on nerinetide content in rat (B) and human (C) plasma. Nerinetide was spiked into the plasma samples at t=0 at a concentration of 65 ug/ml, whereas alteplase (rt-PA) was administered as a 60 min infusion at the indicated concentration (D, E) In-vivo effect of the simultaneous administration of nerinetide and rt-PA on Cmax (D) and AUC (E) in the rat. The nerinetide bolus and alteplase (60 min infusion) were started simultaneously through two separate intravenous lines. Symbols represent mean±SD. Significant differences (in B), (in C) and (in D) are indicated with an asterisk (*) when compared to nerinetide alone group (repeated measures two-way ANOVA with a post hoc Sidak's multiple comparisons test, *P<0.01). Significant difference from nerinetide plus rt-PA (5.4 mg/kg) (in E) are indicated with an asterisk when compared to nerinetide alone group (one-way ANOVA post hoc Tukey's correction for multiple comparisons test, *P<0.01) Sequence identifiers for sequences in FIG. 4A are nerinetide YGRKKRRQRRRKLSSIESDV (SEQ ID NO:3). YGRKKRRQRRRKLSSIESDV (SEQ ID NO:3) (Full-length NA-1, undigested), RRQRRRKLSSIESDV (SEQ ID NO:4), RQRRRKLSSIESDV (SEQ ID NO:5), QRRRKLSSIESDV (SEQ ID NO:6), RRKLSSIESDV (SEQ ID NO:7), RKLSSIESDV (SEQ ID NO:8), KLSSIESDV (SEQ ID NO:9), LSSIESDV (SEQ ID NO:10).

FIGS. 5A-D. Dose separation between nerinetide administration and reperfusion with rt-PA resolves the nullification of the treatment benefit of nerinetide. Nerinetide (7.6 mg/kg) was administered as an intravenous bolus injection either 30 minutes before, or simultaneously with, the onset of a 60-minute infusion of rt-PA (5.4 mg/kg with a 10% bolus followed by 90% over 60 minutes). (A). Experimental timeline. BP=blood pressure. TTC=staining with triphenyl tetrazolium chloride. (B). Hemispheric Infarct volume measurements 24 hours after eMCAo. (C). Percentage of hemispheric brain swelling 24 hours after eMCAo and (D). Neurological scores 24 hours after eMCAo. Treatments were administered intravenously at the times indicated in (A). Bars represent mean±SD, with all individual data points plotted. Significant differences (in B) and (in C) are indicated with an asterisk when compared to the control group/nerinetide alone or with a number sign when compare to the thrombolytic agent (one-way ANOVA post hoc Tukey's correction for multiple comparisons test, *P<0.01 or #P<0.01, respectively) N=12-15 animals/group. Significant differences (in D) are indicated with an asterisk when compared to the control group/nerinetide alone or with a number sign when compare to the thrombolytic agent (Kruskal-Wallis analysis of variance on ranks with a post-hoc Dunn's correction for multiple comparisons test, *P<0.01 or #P<0.01, respectively).

FIGS. 6A-F. D-Tat-L-2B9c has the same target affinity as nerinetide, but is insensitive to cleavage by thrombolytic agents. (A). Nerinetide and D-Tat-L-2B9c have similar binding affinities for the PSD-95 PDZ2 domain. Direct ELISA of the indicated biotinylated peptides to the PDZ2 domain of PSD-95. Nerinetide EC50=0.093 uM. D-Tat-L-2B9c EC50=0.151 uM. Symbols indicate the mean±SD of triplicate experiments. All interactions were titrated multiple times and showed consistent results. (B). Time course of nerinetide (65 ug/ml) or D-TAT-L-2B9c (65 ug/ml) content in PBS during a challenge with rt-PA (135 ug/ml) or plasmin (10 ug/mL). C,D. Time course of nerinetide or D-TAT-L-2B9c content in rat plasma (C) and human plasma (D) during a challenge with rt-PA (135 ug/ml). E,F. Time course of nerinetide or D-TAT-L-2B9c content in rat plasma (E) and human plasma (F) during a challenge with tenecteplase (TNK; 37.5 ug/ml or 6.25 ug/ml, respectively). Significant differences from nerinetide+plasmin (in B), nerinetide+rt-PA (in C,D) and nerinetide+TNK (in E,F) are indicated with an asterisk when compared to the control group/nerinetide alone. (repeated measures two-way ANOVA with a post hoc Sidak's multiple comparisons test, *P<0.01). Symbols are means±SD.

FIGS. 7A-C. Nerinetide and D-TAT-L-2B9c have similar pharmacokinetic profiles. (A). Time course of intravenous bolus administrations of nerinetide (7.6 mg/kg) and D-TAT-L-2B9c (7.6 mg/kg) in the rat. Symbols represent mean±SD. The asterisk (*) represents statistical significance when compared to placebo or control, *P<0.01 by Two-way repeated measures ANOVA with a post hoc Sidak's multiple comparisons test. (B). Area under the concentration—time curve from time zero to 60 min. *P<0.05 by an unpaired student-t test. (C). Comparison of the indicated pharmacokinetic parameters (Cmax=maximum concentration, Tmax=time at which Cmax is reached, T1/2=half-life, AUC (0-last)=area under the curve to last measurement, AUC (0-inf)=AUC extrapolated to infinity, CI=clearance.

FIGS. 8A-D. Concurrent administration of D-Tat-L-2B9c and rt-PA 1 hour after stroke onset reduces infarct volume in animals subjected to eMCAO. A. Experimental timeline. BP=blood pressure. TTC=staining with 2,3,5-tryphenil tetrazolium chloride. B. Infarct volumes, C. Hemispheric swelling and D. Neurological scores 24 hours after eMCAo. D-Tat-L-2B9c and nerinetide were administered intravenously as a bolus injection 60 minutes after eMCAo. Bars represent mean±SD shown, with all individual data points plotted. Significant differences (in B) and (in C) are indicated with an asterisk when compared to the control group/nerinetide alone or with a number sign when compare to the thrombolytic agent (one-way ANOVA post hoc Tukey's correction for multiple comparisons test, *P<0.01 or #P<0.01, respectively) N=10-17 animals/group. Significant differences (in D) are indicated with an asterisk when compared to the control group/nerinetide alone (Kruskal-Wallis analysis of variance on ranks with a post-hoc Dunn's correction for multiple comparisons test, *P<0.01). E. Representative coronal brain slices from the indicated groups stained with 2,3,5-tryphenil tetrazolium chloride (TTC) for infarct volume and hemispheric swelling assessments.

FIG. 9: Plasma levels of nerinetide after administration to healthy humans.

FIGS. 10A-C: Administering alteplase 10 min after the end of 10 min nerinetide infusion substantially reduces cleavage of nerinetide. FIG. 10A, plasma concentration of nerinetide, FIG. 10B, area under curve and FIG. 10C changes of pharmacological parameters.

FIGS. 11A-B: nerinetide is effective over a dosage range of at least 0.025-25 mg/kg in a rat tMCAo model in (A) reducing infarction size and (B) reducing neurologic deficit.

### DEFINITIONS

A “pharmaceutical formulation” or composition is a preparation that permits an active agent to be effective, and lacks additional components which are toxic to the subjects to which the formulation would be administered.

Use of upper case one letter amino acid codes can refer to either D or L amino acids unless the context indicates otherwise. Lower case single letter codes are used to indicate D amino acids. Glycine does not have D and L forms and thus can be represented in either upper or lower case interchangeably.

Numeric values such as concentrations or pH's are given within a tolerance reflecting the accuracy with which the value can be measured. Unless the context requires otherwise, fractional values are rounded to the nearest integer. Unless the context requires otherwise, recitation of a range of values means that any integer or subrange within the range can be used.

The terms “disease” and “condition” are used synonymously to indicate any disruption or interruption of normal structure or function in a subject.

Indicated dosages should be understood as including the margin of error inherent in the accuracy with which dosages can be measured in a typical hospital setting.

The terms “isolated” or “purified” means that the object species (e.g., a peptide) has been purified from contaminants that are present in a sample, such as a sample obtained from natural sources that contain the object species. If an object species is isolated or purified it is the predominant macromolecular (e.g., polypeptide) species present in a sample (i.e., on a molar basis it is more abundant than any other individual species in the composition), and preferably the object species comprises at least about 50 percent (on a molar basis) of all macromolecular species present. Generally, an isolated, purified or substantially pure composition comprises more than 80 to 90 percent of all macromolecular species present in a composition. Most preferably, the object species is purified to essential homogeneity (i.e., contaminant species cannot be detected in the composition by conventional detection methods), wherein the composition consists essentially of a single macromolecular species. The term isolated or purified does not necessarily exclude the presence of other components intended to act in combination with an isolated species. For example, an internalization peptide can be described as isolated notwithstanding that it is linked to an active peptide.

A “peptidomimetic” refers to a synthetic chemical compound which has substantially the same structural and/or functional characteristics of a peptide consisting of natural amino acids. The peptidomimetic can contain entirely synthetic, non-natural analogues of amino acids, or can be a chimeric molecule of partly natural peptide amino acids and partly non-natural analogs of amino acids. The peptidomimetic can also incorporate any amount of natural amino acid conservative substitutions as long as such substitutions also do not substantially alter the mimetic's structure and/or inhibitory or binding activity. Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. In a peptidomimetic of a chimeric peptide comprising an active peptide and an internalization peptide, either the active moiety or the internalization moiety or both can be a peptidomimetic.

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

Excitotoxicity is the pathological process by which neurons and surrounding cells are damaged and killed by the overactivation of receptors for the excitatory neurotransmitter glutamate, such as the NMDA receptors, e.g., NMDA receptors bearing the NMDAR 2B subunit.

The term “subject” includes humans and veterinary animals, such as mammals, as well as laboratory animal models, such as mice or rats used in preclinical studies.

A tat peptide means a peptide comprising or consisting of RKKRRQRRR (SEQ ID NO:11), in which no more than 5 residues are deleted, substituted or inserted within the sequence, which retains the capacity to facilitate uptake of a linked peptide or other agent into cells. Preferably any amino acid changes are conservative substitutions. Preferably, any substitutions, deletions or internal insertions in the aggregate leave the peptide with a net cationic charge, preferably similar to that of the above sequence. Such can be accomplished for example, by not substituting any R or K residues, or retaining the same total of R and K residues. The amino acids of a tat peptide can be derivatized with biotin or similar molecule to reduce an inflammatory response.

Co-administration of pharmacological agents means that the agents are administered sufficiently close in time for detectable amounts of the agents to present in the plasma simultaneously and/or the agents exert a treatment effect on the same episode of disease or the agents act co-operatively, or synergistically in treating the same episode of disease. For example, an anti-inflammatory agent acts cooperatively with an agent including a tat peptide when the two agents are administered sufficiently proximately in time that the anti-inflammatory agent can inhibit an anti-inflammatory response inducible by the internalization peptide.

Statistically significant refers to a p-value that is <0.05, preferably <0.01 and most preferably <0.001.

An episode of a disease means a period when signs and/or symptoms of the disease are present interspersed by flanked by longer periods in which the signs and/or symptoms or absent or present to a lesser extent.

If administration of a drug is not instantaneous, intervals are calculated from or to the initial point of its administration, unless explicitly stated otherwise.

The term “NMDA receptor,” or “NMDAR,” refers to a membrane associated protein that is known to interact with NMDA including the various subunit forms described below. Such receptors can be human or non-human (e.g., mouse, rat, rabbit, monkey).

### DETAILED DESCRIPTION

I. General

The invention is based in part on the observation that the peptide inhibitor of PSD-95, Tat-NR2B9c, and related peptides are cleaved by the serum protease, plasmin, which is induced by thrombolytic agents, such as tPA. If Tat-NR2B9c and a thrombolytic agent are administered together or sufficiently proximal in time to result in substantial overlap of plasma residence between Tat-NR2B9c and plasmin induced by the thrombolytic agent, then cleavage of Tat-NR2B9c can occur, reducing or eliminating its therapeutic effect. Conversely, Tat-NR2B9c has no detrimental effect on the activity of a thrombolytic agent. Inactivation of Tat-NR2B9c by thrombolytic agents can be reduced or avoided by several approaches including spacing the administration of the respective agents to avoid substantial overlap in plasma residence between Tat-NR2B9c and plasmin, using mechanical instead of thrombolytic reperfusion or using active agent that inhibits PSD-95 not subject to cleavage by plasmin, e.g., D-amino acid variants of Tat-NR2B9c.

II. Active Agents

Active agents of the invention specifically bind to PSD-95 (e.g., Stathakism, Genomics 44(1):71-82 (1997)) so as to inhibit its binding to NMDA Receptor 2 subunits including NMDAR2B (e.g., GenBank ID 4099612) and/or NOS (e.g., neuronal or nNOS Swiss-Prot P29475). Preferred peptides inhibit the human forms of PSD-95 NMDAR 2B and NOS for use in a human subject. However, inhibition can also be shown from species variants of the proteins. Such agents can include a PSD-95 peptide inhibitor and an internalization peptide to facilitate passage of the PSD-95 peptide inhibitor across cell membranes and the blood brain barrier. Such agents include an above normal representation of basic residues R and K. When the agents are formed of conventional L amino acids, the overrepresentation of R and K residues renders them particularly susceptible to plasmin cleavage at sites between and R and K residue and the proximate residue on the C-terminal side. Plasmin-sensitivity of nerinetide or other active agents can be demonstrated as in the Examples.

Some peptide inhibitors have an amino acid sequence comprising [E/D/N/Q]-[S/T]-[D/E/Q/N]-[V/L] (SEQ ID NO:1) at their C-terminus. Exemplary peptides comprise: ESDV (SEQ ID NO:12), ESEV (SEQ ID NO:13), ETDV (SEQ ID NO:14), ETAV (SEQ ID NO:15), ETEV (SEQ ID NO:16), DTDV (SEQ ID NO:17), and DTEV (SEQ ID NO:18) as the C-terminal amino acids. Some peptides have an amino acid sequence comprising [I]-[E/D/N/Q]-[S/T]-[D/E/Q/N]-[V/L] (SEQ ID NO:19) at their C-terminus. Exemplary peptides comprise: IESDV (SEQ ID NO:20), IESEV(SEQ ID NO:21), IETDV (SEQ ID NO:22), IETAV (SEQ ID NO:23), IETEV (SEQ ID NO:24), IDTDV (SEQ ID NO:25), and IDTEV (SEQ ID NO:26) as the C-terminal amino acids. Some inhibitor peptides having an amino acid sequence comprising [E/D/N/Q]-[S/T]-[D/E/Q/N]-[V/L] (SEQ ID NO:1) at their C-terminus or X₁-[T/S]-X₂V (SEQ ID NO:2) at the C-terminus, wherein [T/S] are alternative amino acids, X₁ is selected from among E, Q, and A, or an analogue thereof, X₂ is selected from among A, Q, D, N, N-Me-A, N-Me-Q, N-Me-D, and N-Me-N or an analog thereof (see Bach, J. Med. Chem. 51, 6450-6459 (2008) and WO 2010/004003). Some inhibitor peptides having an amino acid sequence comprising X₃-[T/S]-X₄-V (SEQ ID NO:27) at the C-terminus, wherein [T/S] are alternative amino acids, X₃ is selected from among E, D, Q, and A, or an analogue thereof, X₄ is selected from among A, Q, D, E, N, N-Me-A, N-Me-Q, N-Me-D, N-Me-E, and N-Me-N or an analog thereof. Optionally the peptide is N-alkylated in the P3 position (third amino acid from C-terminus, i.e. position occupied by [T/S]). The peptide can be N-alkylated with a cyclohexane or aromatic substituent, and further comprises a spacer group between the substituent and the terminal amino group of the peptide or peptide analogue, wherein the spacer is an alkyl group, preferably selected from among methylene, ethylene, propylene and butylene. The aromatic substituent can be a naphthalen-2-yl moiety or an aromatic ring substituted with one or two halogen and/or alkyl group. Some inhibitor peptides having an amino acid sequence comprising I-X₁-[T/S]-X₂-V (SEQ ID NO:28) at the C-terminus, wherein [T/S] are alternative amino acids, X₁ is selected from among E, Q, and A, or an analogue thereof, X₂ is selected from among A, Q, D, N, N-Me-A, N-Me-Q, N-Me-D, and N-Me-N or an analog thereof. Some inhibitor peptides having an amino acid sequence comprising I-X₃-[T/S]-T₄V (SEQ ID NO:29) at the C-terminus, wherein [T/S] are alternative amino acids, X₃ is selected from among E, Q, A, or D or an analogue thereof, X₄ is selected from among A, Q, D, E, N, N-Me-A, N-Me-Q, N-Me-D, N-Me-E, and N-Me-N or an analog thereof. Exemplary inhibitor peptides have sequences IESDV (SEQ ID NO:20), IETDV (SEQ ID NO:22), KLSSIESDV (SEQ ID NO:9), and KLSSIETDV (SEQ ID NO:30). Inhibitor peptides usually have 3-25 amino acids (without an internalization peptide), peptide lengths of 5-10 amino acids, and particularly 9 amino acids (also without an internalization peptide) are preferred.

Internalization peptides are a well-known class of relatively short peptides that allow many cellular or viral proteins to traverse membranes. They can also promote passage of linked peptides across cell membranes or the blood brain barrier. Internalization peptides, also known as cell membrane transduction peptides, protein transduction domains, brain shuttles or cell penetrating peptides can have e.g., 5-30 amino acids. Such peptides typically have a cationic charge from an above normal representation (relative to proteins in general) of arginine and/or lysine residues that is believed to facilitate their passage across membranes. Some such peptides have at least 5, 6, 7 or 8 arginine and/or lysine residues. Examples include the antennapedia protein (Bonfanti, Cancer Res. 57, 1442-6 (1997)) (and variants thereof), the tat protein of human immunodeficiency virus, the protein VP22, the product of the UL49 gene of herpes simplex virus type 1, Penetratin, SynB1 and 3, Transportan, Amphipathic, gp41NLS, polyArg, and several plant and bacterial protein toxins, such as ricin, abrin, modeccin, diphtheria toxin, cholera toxin, anthrax toxin, heat labile toxins, and Pseudomonas aeruginosa exotoxin A (ETA). Other examples are described in the following references (Temsamani, Drug Discovery Today, 9(23):1012-1019, 2004; De Coupade, Biochem J., 390:407-418, 2005; Saalik Bioconjugate Chem. 15: 1246-1253, 2004; Zhao, Medicinal Research Reviews 24(1):1-12, 2004; Deshayes, Cellular and Molecular Life Sciences 62:1839-49, 2005); Gao, ACS Chem. Biol. 2011, 6, 484-491, SG3 (RLSGMNEVLSFRWL (SEQ ID NO:31)), Stalmans, PLoS ONE 2013, 8(8) e71752, 1-11 and supplement; Figueiredo et al., IUBMB Life 66, 182-194 (2014); Copolovici et al., ACS Nano, 8, 1972-94 (2014); Lukanowski Biotech J. 8, 918-930 (2013); Stockwell, Chem. Biol. Drug Des. 83, 507-520 (2014); Stanzl et al. Accounts. Chem. Res/46, 2944-2954 (2013); Oller-Salvia et al., Chemical Society Reviews 45: 10.1039/c6cs00076b (2016); Behzad Jafari et al., (2019) Expert Opinion on Drug Delivery, 16:6, 583-605 (2019) (all incorporated by reference). Still other strategies use additional methods or compositions to enhance delivery of cargo molecules such as the PSD-95 inhibitors to the brain (Dong, Theranostics 8(6): 1481-1493 (2018).

A preferred internalization peptide is tat from the HIV virus. A tat peptide reported in previous work comprises or consists of the standard amino acid sequence YGRKKRRQRRR (SEQ ID NO:2) found in HIV Tat protein. RKKRRQRRR (SEQ ID NO:11) and GRKKRRQRRR (SEQ ID NO:32) can also be used. If additional residues flanking such a tat motif are present (beside the inhibitor peptide) the residues can be for example natural amino acids flanking this segment from a tat protein, spacer or linker amino acids of a kind typically used to join two peptide domains, e.g., gly (ser)₄ (SEQ ID NO:33), TGEKP (SEQ ID NO:34), GGRRGGGS (SEQ ID NO:35), or LRQRDGERP (SEQ ID NO:36) (see, e.g., Tang et al. (1996), J. Biol. Chem. 271, 15682-15686; Hennecke et al. (1998), Protein Eng. 11, 405-410)), or can be any other amino acids that do not significantly reduce capacity to confer uptake of the variant without the flanking residues. Preferably, the number of flanking amino acids other than an active peptide does not exceed ten on either side of YGRKKRRQRRR (SEQ ID NO:2). However, preferably, no flanking amino acids are present. One suitable tat peptide comprising additional amino acid residues flanking the C-terminus of YGRKKRRQRRR (SEQ ID NO:2) or other inhibitor peptide is YGRKKRRQRRRPQ (SEQ ID NO:37). Other tat peptides that can be used include GRKKRRQRRRPQ (SEQ ID NO:38) and GRKKRRQRRRP (SEQ ID NO:39).

Variants of the above tat peptide having reduced capacity to bind to N-type calcium channels are described by WO2008/109010. Such variants can comprise or consist of an amino acid sequence XGRKKRRQRRR (SEQ ID NO:40), in which X is an amino acid other than Y or can comprise or consist of an amino acid sequence GRKKRRQRRR (SEQ ID NO:32). A preferred tat peptide has the N-terminal Y residue substituted with F. Thus, a tat peptide comprising or consisting of FGRKKRRQRRR (SEQ ID NO:41) is preferred. Another preferred variant tat peptide comprises or consists of GRKKRRQRRR (SEQ ID NO:32). Another preferred tat peptide comprises or consists of RRRQRRKKRG (SEQ ID NO:42) or RRRQRRKKRGY (SEQ ID NO:43). Other tat derived peptides that facilitate uptake of an inhibitor peptide without inhibiting N-type calcium channels include those presented below.

(SEQ ID NO: 41)
X-FGRKKRRQRRR (F-Tat)
(SEQ ID NO: 44)
X-GKKKKKQKKK
(SEQ ID NO: 11)
X-RKKRRQRRR
(SEQ ID NO: 45)
X-GAKKRRQRRR
(SEQ ID NO: 46)
X-AKKRRQRRR
(SEQ ID NO: 47)
X-GRKARRQRRR
(SEQ ID NO: 48)
X-RKARRQRRR
(SEQ ID NO: 49)
X-GRKKARQRRR
(SEQ ID NO: 50)
X-RKKARQRRR
(SEQ ID NO: 51)
X-GRKKRRQARR
(SEQ ID NO: 52)
X-RKKRRQARR
(SEQ ID NO: 53)
X-GRKKRRQRAR
(SEQ ID NO: 54)
X-RKKRRQRAR
(SEQ ID NO: 55)
X-RRPRRPRRPRR
(SEQ ID NO: 56)
X-RRARRARRARR
(SEQ ID NO: 57)
X-RRRARRRARR
(SEQ ID NO: 58)
X-RRRPRRRPRR
(SEQ ID NO: 59)
X-RRPRRPRR
(SEQ ID NO: 60)
X-RRARRARR

X can represent a free amino terminus, one or more amino acids, or a conjugated moiety.

A preferred active agent is Tat-NR2B9c, also known as NA-1 or nerinetide, having the amino acid sequence YGRKKRRQRRRKLSSIESDV (SEQ ID NO:3). Another preferred agent is YGRKKRRQRRRKLSSIETDV (SEQ ID NO:61). All of the amino acids of nerinetide are L-amino acids. Such can also be the case for any of the active agents disclosed above. Thus, nerinetide and other active agents formed of L-amino acids are susceptible to plasmin cleavage.

Some active agents include D-amino acids to reduce or eliminate plasmin-mediated cleavage of a peptide. In such agents, at least the four C-terminal residues of the inhibitor peptide and preferably the five C-terminal residues of the inhibitor peptide are L amino acids, and at least one of the remaining residues in the inhibitor peptide and internalization peptide is a D residue. Positions for inclusion of D residues can be selected such that D residues appear immediately after (i.e., on the C-terminal side) of any basic residue (i.e., arginine or lysine). Plasmin acts by cleaving the peptide bond on the C-terminal side of such basic residues. Inclusion of D residues flanking sites of cleavage, particularly on the C-terminal side of basic residues reduces or eliminates peptide cleavage. Any or all of residues on the C-terminal side of basic residues can be D residues. Any basic residues can also be D amino acids.

Some active agents include at least one D-amino acid in both the internalization peptide and inhibitor peptide. Some active agents include D-amino acids at each position of the internalization peptide. Some active agents include D-amino acids at each position of the inhibitor peptide except the four or five C-terminal residues, which are L-amino acids. Some inhibitor peptides include D-amino acids at each position of the internalization peptide, and each position of the inhibitor peptide except the last four or five C-terminal amino acid residues, which are L-amino acids.

Tat-NR2B9c has the amino acid sequence YGRKKRRQRRRKLSSIESDV (SEQ ID NO:3). Some active agents are variants of this sequence in which ESDV (SEQ ID NO:12) or IESDV (SEQ ID NO:20) are L-amino acids and at least one of the remaining amino acids is a D-amino acid. In some active agents at least the L or K residue at the eighth and ninth position from the C-terminus, or both, is or are D residues. In some active agents, at least one of the R, R, Q, R, R residues occupying the 6^{th}, 7^{th}, 8^{th}, 10^{th,} and 11^{th} positions from the N-terminus is a D residue. In some active agents all of these residues are D-residues. In some active agents, each of residues 4-8 and 10-13 residues are D-amino acids. In some active agents, each of residues 4-13 or 3-13 are D-amino acids. In some active agents, each of the eleven residues of the internalization peptide is a D-amino acid. Some exemplary active agents include ygrkkrrqrrrklssIESDV (SEQ ID NO:62), ygrkkrrqrrrklssIESDV (SEQ ID NO:63), ygrkkrrqrrrklsSIESDV (SEQ ID NO:64), ygrkkrrqrrrkISSIESDV (SEQ ID NO:65), ygrkkrrqrrrkssIESDV (SEQ ID NO:66), ygrkkrrqrrrksIESDV (SEQ ID NO:67), and ygrkkrrqrrrkIETDV (SEQ ID NO:68). Other active agents include variants of the above sequences in which the S at the third position from the C-terminal is replaced with T: ygrkkrrqrrrklssIETDV (SEQ ID NO:69), ygrkkrrqrrrklssIETDV (SEQ ID NO:70), ygrkkrrqrrrklsSIETDV (SEQ ID NO:71), ygrkkrrqrrrkISSIETDV (SEQ ID NO:72), ygrkkrrqrrrkssIETDV (SEQ ID NO:73), ygrkkrrqrrrksIETDV (SEQ ID NO:74), and ygrkkrrqrrrkIETDV (SEQ ID NO:75). Active agents include ygrkkrrqrrrIESDV (SEQ ID NO:76) (D-Tat-L-2B5c) and ygrkkrrqrrrIETDV (SEQ ID NO:77).

The invention also includes an active agent comprising an internalization peptide linked, e.g., as a fusion peptide, to an inhibitor peptide, which inhibits PSD-95 binding to NOS, wherein the internalization peptide has an amino acid sequence comprising YGRKKRRQRRR (SEQ ID NO:2), GRKKRRQRRR (SEQ ID NO:32), or RKKRRQRRR (SEQ ID NO:11) and the inhibitor peptide has a sequence comprising KLSSIESDV (SEQ ID NO:9), or a variant thereof with up to 1, 2, 3, 4, or 5 substitutions or deletions total in the internalization peptide and inhibitor peptide. In such active agents at least the four or five C-terminal amino acids of the inhibitor peptide are L-amino acids, and a contiguous segment of amino acids including all of the R and K residues and the residue immediately C-terminal to the most C-terminal R or K residue are D-amino acids. Thus, in a peptide having the sequence YGRKKRRQRRRKLSSIESDV (SEQ ID NO:3), a contiguous segment from the first R to the L residue are D-amino acids.

One example of permitted substitutions is provided by the motif [E/D/N/Q]-[S/T]-[D/E/Q/N]-[V/L] (SEQ ID NO:1) at the C-terminus of the inhibitor peptide. For example, the third amino acid from the C-terminus can be S or T. Preferably each of the five C-terminal amino acids of the inhibitor peptide are L-amino acids. Preferably every other amino acid is a D-amino acid as in the active agent ygrkkrrqrrrklssIESDV (SEQ ID NO:78), wherein the lower case letter are D-amino acids and the upper case letters are L-amino acids.

Preferred active agents with D-amino acids have enhanced stability in rat or human plasma (e.g., by half-life) compared with Tat-NR2B9c or an otherwise identical all L-active agent. Stability can be measured as in the examples. Preferred active have enhanced plasmin resistance compared with Tat-NR2B9c or an otherwise identical all L active agent. Plasmin resistance can be measured as in the examples. Active agents preferably bind to PSD-95 within 1.5-fold, 2-fold, 3 fold or 5-fold of Tat-NR2B9c (all L) or an otherwise identical all L peptide or have indistinguishable binding within experimental error. Preferred active agents compete for binding with Tat-NR2B9c or a peptide containing the last 15-20 amino acids of a NMDA Receptor subunit 2 sequence that contains the PDZ binding domain, for binding to PSD-95 (e.g., a ten-fold excess of active agent reduces Tat-NR2B9c binding) by at least 10%, 25% or 50%. Competition provides an indication that the active agent binds to the same or overlapping binding site as Tat-NR2B9c. Possession of the same or overlapping binding sites can also be shown by alanine mutagenesis of PSD-95. If mutagenesis of the same or overlapping set of residues reduces binding of an active agent and Tat-NR2B9c, then the active agent and TAT-NR2B9c bind to the same or overlapping sites on PSD-95.

Active agents of the invention can contain modified amino acid residues for example, residues that are N-alkylated. N-terminal alkyl modifications can include e.g., N-Methyl, N-Ethyl, N-Propyl, N-Butyl, N-Cyclohexylmethyl, N-Cyclyhexylethyl, N-Benzyl, N-Phenylethyl, N-phenylpropyl, N-(3, 4-Dichlorophenyl)propyl, N-(3,4-Difluorophenyl)propyl, and N-(Naphthalene-2-yl)ethyl). Active agents can also include retro peptides. A retro peptide has a reverse amino acid sequence. Peptidomimetics also include retro inverso peptides in which the order of amino acids is reversed from so the originally C-terminal amino acid appears at the N-terminus and D-amino acids are used in place of L-amino (e.g., acids vdseisslkrrrqrrkkrgy (SEQ ID NO:79), also known as RI-NA-1).

Appropriate pharmacological activity of peptides, peptidomimetics or other agent can be confirmed if desired, using previously described rat models of stroke before testing in the primate and clinical trials described in the present application. Peptides or peptidomimetics can also be screened for capacity to inhibit interactions between PSD-95 and NMDAR 2B using assays described in e.g., US 20050059597, which is incorporated by reference. Useful peptides or other agents typically have IC50 values of less than 50 μM, 25 μM, 10 μM, 0.1 μM or 0.01 μM in such an assay. Preferred peptides or other agents typically have an IC50 value of between 0.001-1 μM, and more preferably 0.001-0.05, 0.05-0.5 or 0.05 to 0.1 μM. When a peptide or other agent is characterized as inhibiting binding of one interaction, e.g., PSD-95 interaction to NMDAR2B, such description does not exclude that the peptide or agent also inhibits another interaction, for example, inhibition of PSD-95 binding to nNOS.

Peptides such as those just described can optionally be derivatized (e.g., acetylated, phosphorylated, myristoylated, geranylated, pegylated and/or glycosylated) to improve the binding affinity of the inhibitor, to improve the ability of the inhibitor to be transported across a cell membrane or to improve stability. As a specific example, for inhibitors in which the third residue from the C-terminus is S or T, this residue can be phosphorylated before use of the peptide.

Internalization peptides can be attached to inhibitor peptides by conventional methods. For example, the agents can be joined to internalization peptides by chemical linkage, for instance via a coupling or conjugating agent. Numerous such agents are commercially available and are reviewed by S. S. Wong, Chemistry of Protein Conjugation and Cross-Linking, CRC Press (1991). Some examples of cross-linking reagents include J-succinimidyl 3-(2-pyridyldithio) propionate (SPDP) or N,N′-(1,3-phenylene) bismaleimide; N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents include p,p′-difluoro-m, m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

A linker, e.g., a polyethylene glycol linker, can be used to dimerize the active moiety of the peptide or the peptidomimetic to enhance its affinity and selectivity towards proteins containing tandem PDZ domains. See e.g., Bach et al., (2009) Angew. Chem. Int. Ed. 48:9685-9689 and WO 2010/004003. A PL motif-containing peptide is preferably dimerized via joining the N-termini of two such molecules, leaving the C-termini free. Bach further reports that a pentamer peptide IESDV (SEQ ID NO:20) from the C-terminus of NMDAR 2B was effective in inhibiting binding of NMDAR 2B to PSD-95. IETDV (SEQ ID NO:22) can also be used instead of IESDV (SEQ ID NO:20). Optionally, about 2-10 copies of a PEG can be joined in tandem as a linker. Optionally, the linker can also be attached to an internalization peptide or lipidated to enhance cellular uptake. Examples of illustrative dimeric inhibitors are shown below (see Bach et al., PNAS 109 (2012) 3317-3322). Any of the PSD-95 inhibitors disclosed herein can be used instead of IETDV (SEQ ID NO:22), and any internalization peptide or lipidating moiety can be used instead of tat. Other linkers to that shown can also be used.

Internalization peptides can also be linked to inhibitor peptide as fusion peptides, preferably with the C-terminus of the internalization peptide linked to the N-terminus of the inhibitor peptide leaving the inhibitor peptide with a free C-terminus.

Instead of or as well as linking a peptide to an internalization peptide, such a peptide can be linked to a lipid (lipidation) to increase hydrophobicity of the conjugate relative to the peptide alone and thereby facilitate passage of the linked peptide across cell membranes and/or across the brain barrier. Lipidation is preferably performed on the N-terminal amino acid but can also be performed on internal amino acids, provided the ability of the peptide to inhibit interaction between PSD-95 and NMDAR 2B is not reduced by more than 50%. Preferably, lipidation is performed on an amino acid other than one of the five most C-terminal amino acids. Lipids are organic molecules more soluble in ether than water and include fatty acids, glycerides and sterols. Suitable forms of lipidation include myristoylation, palmitoylation or attachment of other fatty acids preferably with a chain length of 10-20 carbons, such as lauric acid and stearic acid, as well as geranylation, geranylgeranylation, and isoprenylation. Lipidations of a type occurring in posttranslational modification of natural proteins are preferred. Lipidation with a fatty acid via formation of an amide bond to the alpha-amino group of the N-terminal amino acid of the peptide is also preferred. Lipidation can be by peptide synthesis including a prelipidated amino acid, be performed enzymatically in vitro or by recombinant expression, by chemical crosslinking or chemical derivatization of the peptide. Amino acids modified by myristoylation and other lipid modifications are commercially available. Use of a lipid instead of an internalization peptide reduces the number of K and R residues providing sites of plasmin cleavage. Some exemplary lipidated molecules include KLSSIESDV (SEQ ID NO:9), kISSIESDV (SEQ ID NO:80), ISSIESDV (SEQ ID NO:81), LSSIESDV (SEQ ID NO:10), SSIESDV (SEQ ID NO:82), SIESDV (SEQ ID NO:83), IESDV (SEQ ID NO:20), KLSSIETDV (SEQ ID NO:29), kISSIETDV (SEQ ID NO:84), ISSIETDV (SEQ ID NO:85), LSSIETDV (SEQ ID NO:86), SSIETDV (SEQ ID NO:87), SIETDV (SEQ ID NO:88), IETDV (SEQ ID NO:22) with lipidation preferably at the N-terminus.

Inhibitor peptides, optionally fused to internalization peptides, can be synthesized by solid phase synthesis or recombinant methods. Peptidomimetics can be synthesized using a variety of procedures and methodologies described in the scientific and patent literature, e.g., Organic Syntheses Collective Volumes, Gilman et al. (Eds) John Wiley & Sons, Inc., NY, al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

III. Salts

Peptides of the type described above are typically made by solid state synthesis. Because solid state synthesis uses trifluoroacetate (TFA) to remove protecting groups or remove peptides from a resin, peptides are typically initially produced as trifloroacetate salts. The trifluoroacetate can be replaced with another anion by for example, binding the peptide to a solid support, such as a column, washing the column to remove the existing counterion, equilibrating the column with a solution containing the new counterion and then eluting the peptide, e.g., by introducing a hydrophobic solvent such as acetonitrile into the column. Replacement of trifluoroacetate with acetate can be done with an acetate wash as the last step before peptide is eluted in an otherwise conventional solid state synthesis. Replacing trifluoroacetate or acetate with chloride can be done with a wash with ammonium chloride followed by elution. Use of a hydrophobic support is preferred and preparative reverse phase HPLC is particularly preferred for the ion exchange. Trifluoroacetate can be replaced with chloride directly or can first be replaced by acetate and then the acetate replaced by chloride.

Counterions, whether trifluoroacetate, acetate or chloride, bind to positively charged atoms on Tat-NR2B9c and D-variants thereof, particularly the N-terminal amino group and amino side chains arginine and lysine residues. Although practice of the invention, it is not dependent on understanding the exact stoichiometry of peptide to anion in a salt of Tat-NR2B9c and its D-variants, it is believed that up to about 9 counterion molecules are present per molecule of salt.

Although replacement of one counterion by another takes place efficiently, the purity of the final counterion may be less than 100%. Thus, reference to a chloride salt of Tat-NR2B9c or its D-variants described herein means that in a preparation of the salt, chloride is the predominant anion by weight (or moles) over all other anions present in the aggregate in the salt. In other words, chloride constitutes greater than 50% and preferably greater than 75%, 95%, 99%, 99.5% or 99.9% by weight or moles of the all anions present in the salt or formulation. In such a salt or formulation prepared from the salt, acetate and trifluoroacetate in combination and individually constitutes less than 50%, 25%, 5%, 1%, 0.5% or 0.1 of the anions in the salt or formulation by weight or moles.

IV. Formulations

Active agents can be incorporated into liquid formulation or lyophilized formulations. A liquid formulation can include a buffer, salt and water. A preferred buffer is sodium phosphate. A preferred salt is sodium chloride. The pH can be e.g., pH7.0 or about physiological.

Lyophilized formulations can be prepared from a prelyophilized formulation comprising an active agent, a buffer, a bulking agent and water. Other components, such as cryo or lyopreservatives, a tonicity agent pharmaceutically acceptable carriers and the like may or may be present. A preferred buffer is histidine. A preferred bulking agent is trehalose. Trehalose also serves as a cryo and lyo-preservative. An exemplary prelyophilized formulation comprises the active agent, histidine (10-100 mM, 15-100 mM 15-80 mM, 40-60 mM or 15-60 mM, for example, 20 mM or optionally 50 mM, or 20-50 mM)) and trehalose (50-200 mM, preferably 80-160 mM, 100-140 mM, more preferably 120 mM). The pH is 5.5 to 7.5, more preferably, 6-7, more preferably 6.5. The concentration of active agent is 20-200 mg/ml, preferably 50-150 mg/ml, more preferably 70-120 mg/ml or 90 mg/ml. Thus, an exemplary prelyophilized formulation is 20 mM histidine, 120 mM trehalose, and 90 mg/ml chloride salt of active agent. Optionally an acetylation scavenger, such as lysine can be included, as described in U.S. Pat. No. 10,206,878, to further reduce any residual acetate or trifluoroacetate in the formulation.

After lyophilization, lyophilized formulations have a low-water content, preferably from about 0%-5% water, more preferably below 2.5% water by weight. Lyophilized formulations can be stored in a freezer (e.g., −20 or −70° C.), in a refrigerator (0-40° C.) or at room temperature (20-25° C.).

Active agents can be reconstituted in an aqueous solution, preferably water for injection or optionally normal saline (0.8-1.0% saline and preferably 0.9% saline). Reconstitution can be to the same or a smaller or larger volume than the prelyophilized formulation. Preferably, the volume is larger post-reconstitution than before (e.g., 3-6 times larger). For example, a prelyophilization volume of 3-5 ml can be reconstituted as a volume of 10 mL, 12 mL, 13.5 ml, 15 mL or 20 mL or 10-20 mL among others. After reconstitution, the concentration of histidine is preferably 2-20 mM, e.g., 2-7 mM, 4.0-6.5 mM, 4.5 mM or 6 mM; the concentration of trehalose is preferably 15-45 mM or 20-40 mM or 25-27 mM or 35-37 mM. The concentration of lysine is preferably 100-300 mM, e.g., 150-250 mM, 150-170 mM or 210-220 mM. The active agent is preferably at a concentration of 10-30 mg/ml, for example 15-30, 18-20, 20 mg/ml of active agent or 25-30, 26-28 or 27 mg/mL active agent. An exemplary formulation after reconstitution has 4-5 mM histidine, 26-27 mM trehalose, 150-170 mM lysine and 20 mg/ml active agent (with concentrations rounded to the nearest integer). A second exemplary formulation after reconstitution has 5-7 mM histidine, 35-37 mM trehalose, 210-220 mM lysine and 26-28 mg/ml active agent (with concentrations rounded to the nearest integer). The reconstituted formulation can be further diluted before administration such as by adding into a fluid bag containing normal saline.

V. Conditions

The present methods are useful for treating conditions resulting from ischemia, particularly ischemia of the CNS, and more particularly ischemic stroke, such as acute ischemic stroke. Treatment with a thrombolytic agent or mechanical reperfusion acts to remove a blockage in a blood vessel causing ischemia. Treatment with active agents inhibiting PSD-95 acts to reduce damaging effects of ischemia.

A stroke is a condition resulting from impaired blood flow in the CNS regardless of cause. Potential causes include embolism, hemorrhage and thrombosis. Some neuronal cells die immediately as a result of impaired blood flow. These cells release their component molecules including glutamate, which in turn activates NMDA receptors, which raise intracellular calcium levels, and intracellular enzyme levels leading to further neuronal cell death (the excitotoxicity cascade). The death of CNS tissue is referred to as infarction. Infarction Volume (i.e., the volume of dead neuronal cells resulting from stroke in the brain) can be used as an indicator of the extent of pathological damage resulting from stroke. The symptomatic effect depends both on the volume of an infarction and where in the brain it is located. Disability index can be used as a measure of symptomatic damage, such as the Rankin Stroke Outcome Scale (Rankin, Scott Med J; 2:200-15 (1957)) and the Barthel Index. The Rankin Scale is based on assessing directly the global conditions of a subject as follows.
0: No symptoms at all
1: No significant disability despite symptoms; able to carry out all usual duties and activities.
2: Slight disability; unable to carry out all previous activities but able to look after own affairs without assistance.
3: Moderate disability requiring some help, but able to walk without assistance
4: Moderate to severe disability; unable to walk without assistance and unable to attend to own bodily needs without assistance.
5: Severe disability; bedridden, incontinent, and requiring constant nursing care and attention.

The Barthel Index is based on a series of questions about the subject's ability to carry out 10 basic activities of daily living resulting in a score between 0 and 100, a lower score indicating more disability (Mahoney et al, Maryland State Medical Journal 14:56-61 (1965)).

Alternatively stroke severity/outcomes can be measured using the NIH stroke scale, available at world wide web ninds.nih.gov/doctors/NIH Stroke ScaleJBooklet.pdf.

The scale is based on the ability of a subject to carry out 11 groups of functions that include assessments of the subject's level of consciousness, motor, sensory and language functions.

An ischemic stroke refers more specifically to a type of stroke that caused by blockage of blood flow to the brain. The underlying condition for this type of blockage is most commonly the development of fatty deposits lining the vessel walls. This condition is called atherosclerosis. These fatty deposits can cause two types of obstruction. Cerebral thrombosis refers to a thrombus (blood clot) that develops at the clogged part of the vessel “Cerebral embolism” refers generally to a blood clot that forms at another location in the circulatory system, usually the heart and large arteries of the upper chest and neck. A portion of the blood clot then breaks loose, enters the bloodstream and travels through the brain's blood vessels until it reaches vessels too small to let it pass. A second important cause of embolism is an irregular heartbeat, known as arterial fibrillation. It creates conditions in which clots can form in the heart, dislodge and travel to the brain. Additional potential causes of ischemic stroke are hemorrhage, thrombosis, dissection of an artery or vein, a cardiac arrest, shock of any cause including hemorrhage, and iatrogenic causes such as direct surgical injury to brain blood vessels or vessels leading to the brain or cardiac surgery. Ischemic stroke accounts for about 83 percent of all cases of stroke.

Transient ischemic attacks (TIAs) are minor or warning strokes. In a TIA, conditions indicative of an ischemic stroke are present and the typical stroke warning signs develop. However, the obstruction (blood clot) occurs for a short time and tends to resolve itself through normal mechanisms. Patients undergoing heart surgery are at particular risk of transient cerebral ischemic attack.

Hemorrhagic stroke accounts for about 17 percent of stroke cases. It results from a weakened vessel that ruptures and bleeds into the surrounding brain. The blood accumulates and compresses the surrounding brain tissue. The two general types of hemorrhagic strokes are intracerebral hemorrhage and subarachnoid hemorrhage. Hemorrhagic stroke result from rupture of a weakened blood vessel ruptures. Potential causes of rupture from a weakened blood vessel include a hypertensive hemorrhage, in which high blood pressure causes a rupture of a blood vessel, or another underlying cause of weakened blood vessels such as a ruptured brain vascular malformation including a brain aneurysm, arteriovenous malformation (AVM) or cavernous malformation. Hemorrhagic strokes can also arise from a hemorrhagic transformation of an ischemic stroke which weakens the blood vessels in the infarct, or a hemorrhage from primary or metastatic tumors in the CNS which contain abnormally weak blood vessels. Hemorrhagic stroke can also arise from iatrogenic causes such as direct surgical injury to a brain blood vessel. An aneurysm is a ballooning of a weakened region of a blood vessel. If left untreated, the aneurysm continues to weaken until it ruptures and bleeds into the brain. An arteriovenous malformation (AVM) is a cluster of abnormally formed blood vessels. A cavernous malformation is a venous abnormality that can cause a hemorrhage from weakened venous structures. Any one of these vessels can rupture, also causing bleeding into the brain. Hemorrhagic stroke can also result from physical trauma. Hemorrhagic stroke in one part of the brain can lead to ischemic stroke in another through shortage of blood lost in the hemorrhagic stroke.

One subject class amenable to treatments are subjects undergoing a surgical procedure that involves or may involve a blood vessel supplying the brain, or otherwise on the brain or CNS. Some examples are subjects undergoing cardiopulmonary bypass, carotid stenting, diagnostic angiography of the brain or coronary arteries of the aortic arch, vascular surgical procedures and neurosurgical procedures. Additional examples of such subjects are discussed in section IV above. Patients with a brain aneurysm are particularly suitable. Such subjects can be treated by a variety of surgical procedures including clipping the aneurysm to shut off blood, or performing endovascular surgery to block the aneurysm with small coils or introduce a stent into a blood vessel from which an aneurysm emerges, or inserting a microcatheter. Endovascular procedures are less invasive than clipping an aneurysm and are associated with a better subject outcome but the outcome still includes a high incidence of small infarctions. Such subjects can be treated with an inhibitor of PSD95 interaction with NMDAR 2B and particularly the active agents described above. The timing of administration relative to performing surgery can be as described above for the clinical trial.

Another class of subjects is those with ischemic strokes who are candidates for endovascular thrombectomy to remove the clot, such as the ESCAPE-NA1 trial (NCT02930018). Drug can be administered before or after the surgery to remove the clot, and is expected to improve outcome from both the stroke itself and any potential iatrogenic strokes associated with the procedures as discussed supra. Another example is those who have been diagnosed with a potential stroke without the use of imaging criteria and receive treatment within hours of the stroke, preferably within the first 3 hours but optionally the first 6, 9 or 12 hour after stroke onset (similar to NCT02315443).

VI. Co-Administration of Active Agents Inhibiting PSD-95 with Reperfusion

Plaques and blood clots (also known as emboli) causing ischemia can be dissolved, removed or bypassed by both pharmacological and physical means. The dissolving, removal of plaques and blood clots and consequent restoration of blood flow is referred to as reperfusion. One class of agents acts by thrombolysis. Thrombolytic agents work by promoting production of plasmin from plasminogen. Plasmin clears cross-linked fibrin mesh (the backbone of a clot), making the clot soluble and subject to further proteolysis by other enzymes, and restores blood flow in occluded blood vessels. Examples of thrombolytic agents include tissue plasminogen activator t-PA, alteplase (ACTIVASE®), reteplase (RETAVASE®), tenecteplase (TNKase®), anistreplase (EMINASE®), streptokinase (KABIKINASE®, STREPTASE®), and urokinase (ABBOKINASE®).

Another class of drugs that can be used for reperfusion is vasodilators. These drugs act by relaxing and opening up blood vessels thus allowing blood to flow around an obstruction. Some examples of types of vasodilator agents include alpha-adrenoceptor antagonists (alpha-blockers), Angiotensin receptor blockers (ARBs), β₂-adrenoceptor agonists, calcium-channel blockers (CCBs), centrally acting sympatholytics, direct acting vasodilators, endothelin receptor antagonists, ganglionic blockers, nitrodilators, phosphodiesterase inhibitors, potassium-channel openers, and renin inhibitors.

Another class of drugs that can be used for reperfusion is hypertensive agents (i.e., drugs raising blood pressure), such as epinephrine, phenylephrine, pseudoephedrine, norepinephrine; norephedrine; terbutaline; salbutamol; and methylephedrine. Increased perfusion pressure can increase flow of blood around an obstruction.

Mechanical methods of reperfusion include angioplasty, catheterization, and artery bypass graft surgery, stenting, embolectomy, endarterectomy or endovascular thrombectomy. Such procedures restore plaque flow by mechanical removal of a plaque, holding a blood vessel open, so blood can flow around a plaque or bypassing a plaque.

Other mechanical methods of reperfusion include use of a device that diverts blood flow from other areas of the body to the brain. An example is a catheter partially occluding the aorta, such as the CoAxia NeuroFlo™ catheter device, which has recently been subjected to a randomized trial and may get FDA approval for stroke treatment. This device has been used on subjects presenting with stroke up to 14 hours after onset of ischemia.

The present methods provide regimes for administering both reperfusion and an active agent inhibiting PSD-95, such that they can both contribute to treatment. Such regimes avoid administering an active agent inhibiting PSD-95 sensitive to plasmin cleavage (e.g., all L-amino acids) and a thrombolytic agent sufficiently proximal in time that there is substantial co-residency in the plasma of both the active agent that inhibits PSD-95 and plasmin induced by the thrombolytic agent resulting in cleavage of the active agent that inhibits PSD-95 and reduced or eliminated activity of the active agent that inhibits PSD-95. Although in much of the description that follows Tat-NR2B9c is referred to as an exemplary, the same methods should be understood as referring to other active agents inhibiting PSD-95 as described herein.

Tat-NR2B9c has a plasma half-life in human plasma of about ten minutes. This does not mean that Tat-NR2B9c is normally half-degraded after ten minutes in plasma, but rather than Tat-NR2B9c is moved out of the plasma with a half-life of ten minutes. Alteplase (a recombinant form of tPA) has a half-life in human plasma of only about five minutes. But more significant for present purposes is the half-life of plasmin, which is induced by alteplase and other thrombolytic agents and is responsible for cleavage of Tat-NR2B9c. Plasmin has been reported to have a half-life in human plasma of about 4-8 hr.

It follows from the respective half-lives of Tat-NR2B9c and plasmin that interaction between the two can be avoided by administering Tat-NR2B9c at least one plasma half-life of Tat-NR2B9c (i.e., about ten minutes) before administering the thrombolytic agent. A greater interval of 2 or 3 half-lives, such that Tat-NR2B9c is administered at least 20 or 30 minutes before a thrombolytic agent still further reduces co-residency in the plasma and consequent potential for inactivation of Tat-NR2B9c and the thrombolytic agent. Administering Tat-NR2B9c even further in advance of a thrombolytic agent, such as at least 45 min, 1 hr, 2 hr, 3 hr, 5 hr reduces potential for inactivation of Tat-NR2B9c still further. For administration of Tat-NR2B9c over a 10 min period as is typical, a period of 20 minutes from the start of Tat-NR2B9c administration is equivalent to 10 minutes from the end of Tat-NR2B9c administration and a period 30 minutes from the start of Tat-NR2B9c administration is equivalent to 20 minutes from the end.

A plasmin-sensitive active agent inhibiting PSD-95 and a thrombolytic agent should not be administered together either as a single composition or co-administered at the same time as separate compositions.

If a thrombolytic agent is administered first then sufficient time should be allowed to elapse before administering an active agent inhibiting PSD-95, which is sensitive to plasmin cleavage, that the plasma concentration of plasmin induced by the thrombolytic agent has significantly reduced. For example, the interval, can be at least 3 hr, 4 hr, 8 hr, 12 hr or 24 hours.

Mechanical methods of reperfusion or reperfusion induced by classes of drugs other than thrombolytic agents can be performed at any time with respect to administration of an active agent inhibiting PSD-95 without any inactivation of the active agent occurring. Such is also the case for administration of D-variants of active agents inhibiting PSD-95 resistant to plasmin cleavage. Cleavage of an active agent inhibiting PSD-95 can also be reduced by administering it by a route that allows it to reach the brain without passing through the blood, for example, non-intravenous, such as by intranasal or intrathecal administration.

In subjects with or suspected of having ischemia, who have not yet received any treatment, and in which the relative order of treatments can be controlled, it is usually preferable to treat with an active agent inhibiting PSD-95 first and then wait a suitable interval as discussed above to administer a thrombolytic agent notwithstanding conventional wisdom in the field that thrombolytic agents should be administered as soon as possible to mitigate on-going death of neuronal cells, and at least before 3 hours or 4.5 hour after onset of stroke. The interval between administering an active agent inhibiting PSD-95 and a thrombolytic agent can be used for performing additional testing to confirm presence of ischemic stroke and eliminate presence or risk of hemorrhagic stroke or other hemorrhage for which administration of a thrombolytic agent would be counter-indicated. Prior administration of the active agent inhibiting PSD-95 also had the advantage of prolonging the window in which the thrombolytic agent is likely to be effective after onset of ischemia. In the absence of an active agent inhibiting PSD-95 the window is only about 3-4.5 hr but it can be prolonged by an active agent inhibiting PSD-95 at least 5, 6, 9, 12 or 24 hours.

Even if it has already been determined that a subject has ischemic stroke and is eligible for treatment with a thrombolytic agent (e.g., lack of hemorrhage), then it is still preferable to administer an active agent that inhibits PSD-95 and is sensitive to plasmin-cleavage at an interval of at least 10, 20, 30, 40, 50, 60, 120, or 180 minutes before the thrombolytic agent even if this means the thrombolytic agent is administered after the 3 or 4.5 hour time point beyond which conventional wisdom would consider it ineffective.

If, however, waiting to administer reperfusion is considered to present an unacceptable risk of reducing its efficacy, reperfusion can be effected by mechanical reperfusion or with a class of drugs other than thrombolytic agents, such as vasodilators or hypertensive agents.

In subjects with ischemia, who have already received a thrombolytic agent, then there should be a suitable interval of at least about 3 hr as discussed above before administering a an active agent inhibiting PSD-95 subject to cleavage by plasmin. Alternatively, if this interval is not deemed acceptable due to e.g., deterioration of a subject's condition that would occur during the interval, then an active agent inhibiting PSD-95 resistant to plasmin cleavage can be used.

Thus a population of subjects undergoing treatment for ischemia receiving both an active agent inhibiting PSD-95 and reperfusion can include individuals receiving different forms of treatment. Such a population can represent for example subjects treated by the same physician or by the same institution. Such a population can include at least 10, 50, 100 or 500 subjects. Some subjects in such a population receive an active agent inhibiting PSD-95 and mechanical reperfusion or treatment with a vasodilator or hypertensive agent to effect reperfusion. Such forms of reperfusion can be performed in any sequence with administration of the active agent inhibiting PSD-95. Some subjects in the population receive an active agent inhibiting PSD-95 sensitive to plasmin cleavage and a thrombolytic agent, wherein the active agent inhibiting PSD-95 is administered at least 10, 20, 30, 40, 50, 60, 120 or 180 minutes before the thrombolytic agent. No subjects in such a population receive a thrombolytic agent less than 3 hours before or less than 10, 20, 30, 40, 50, 60, 120 or 180 minutes after they receive an active agent inhibiting PSD-95. Some populations have no subjects in which the thrombolytic agent is administered before the active agent inhibiting PSD-95. Some populations lack subjects in which the thrombolytic agent is administered less than 30 minutes after the administration of the active agent inhibiting inhibitor. Some populations include subjects administered the active agent inhibiting PSD-95 and mechanical reperfusion without receiving a thrombolytic agent. Some populations consist of (a) subjects administered the active agent inhibiting PSD-95 and mechanical reperfusion without a thrombolytic agent; and (b) subjects administered the active agent inhibiting PSD-95 and a thrombolytic agent, wherein the thrombolytic agent is administered at least 10 minutes after the active agent inhibiting PSD-95. Optionally at least some of the subjects of (b) also are administered mechanical reperfusion.

Alternatively if both an active agent inhibiting PSD-95 sensitive to plasmin cleavage and a different active agent inhibiting PSD-95 resistant to plasmin cleavage are available a population of individuals having or at risk of ischemia can include subjects administered a first active agent inhibiting PSD-95 cleavable by plasmin and a thrombolytic agent, wherein the first active agent inhibiting PSD-95 is administered an interval of at least 10, 20, 30, 40, 50, 60, 120 or 180 minutes before the thrombolytic agent; and subjects administered a second active agent inhibiting PSD-95 resistant to cleavage by plasmin and a thrombolytic agent, wherein the thrombolytic agent is administered before or within the interval after the second active agent that inhibits PSD-95.

Both treatment with an active agent and reperfusion therapy independently have ability to reduce infarction size and functional deficits due to ischemia. When used in combination according to the present methods, the reduction in infarction size and/or functional deficits is preferably greater than that from use of either agent or procedure alone administered under a comparable regime other than for the combination (i.e., co-operative). More preferably, the reduction in infarction side and/or functional deficits is at least additive or preferably more than additive (i.e., synergistic) of reductions achieved by the agents (or reperfusion procedure) alone under a comparable regime except for the combination. In some regimes, the reperfusion therapy is effective in reducing infarction size and/or functional times at a time post onset of ischemia (e.g., more than 4.5 hr) when it would be ineffective but for the concurrent or prior administration of the active agent inhibiting PSD-95. Put another way, when a subject is administered an active agent and reperfusion therapy, the reperfusion therapy is preferably at least as effective as it would be if administered at an earlier time without the active agent. Thus, the active agent effectively increases the efficacy of the reperfusion therapy by reducing one or more damaging effects of ischemia before or as reperfusion therapy takes effects. The active agent can thus compensate for delay in administering the reperfusion therapy whether the delay be from delay in the subject recognizing the danger of his or her initial symptoms delays in transporting a subject to a hospital or other medical institution or delays in performing diagnostic procedures to establish presence of ischemia and/or absence of hemorrhage or unacceptable risk thereof. Statistically significant combined effects of an active agent and reperfusion therapy including additive or synergistic effects can be demonstrated between populations in a clinical trial or between populations of animal models in preclinical work.

VII. Effective Regimes of Administration

An active agent is administered in an amount, frequency and route of administration effective to cure, reduce or inhibit further deterioration of at least one sign or symptom of a disease in a subject having the disease being treated. A therapeutically effective amount (before administration) or therapeutically effective plasma concentration after administration means an amount or level of active agent sufficient significantly to cure, reduce or inhibit further deterioration of at least one sign or symptom of the disease or condition to be treated in a population of subjects (or animal models) suffering from the disease treated with an agent of the invention relative to the damage in a control population of subjects (or animal models) suffering from that disease or condition who are not treated with the agent. The amount or level is also considered therapeutically effective if an individual treated subject achieves an outcome more favorable than the mean outcome in a control population of comparable subjects not treated by methods of the invention. A therapeutically effective regime involves the administration of a therapeutically effective dose at a frequency and route of administration needed to achieve the intended purpose.

For a subject suffering from stroke or other ischemic condition, the active agent is administered in a regime comprising an amount frequency and route of administration effective to reduce the damaging effects of stroke or other ischemic condition. When the condition requiring treatment is stroke, the outcome can be determined by infarction volume or disability index, and a dosage is considered therapeutically effective if an individual treated subject shows a disability of two or less on the Rankin scale and 75 or more on the Barthel scale, or if a population of treated subjects shows a significantly improved (i.e., less disability) distribution of scores on a disability scale than a comparable untreated population, see Lees et al., N. Engl. J. Med. 2006; 354:588-600. A single dose of agent can be sufficient for treatment of stroke.

The invention also provides methods and formulations for the prophylaxis of a disorder in a subject at risk of that disorder. Usually such a subject has an increased likelihood of developing the disorder (e.g., a condition, illness, disorder or disease) relative to a control population. The control population for instance can comprise one or more individuals selected at random from the general population (e.g., matched by age, gender, race and/or ethnicity) who have not been diagnosed or have a family history of the disorder. A subject can be considered at risk for a disorder if a “risk factor” associated with that disorder is found to be associated with that subject. A risk factor can include any activity, trait, event or property associated with a given disorder, for example, through statistical or epidemiological studies on a population of subjects. A subject can thus be classified as being at risk for a disorder even if studies identifying the underlying risk factors did not include the subject specifically. For example, a subject undergoing heart surgery is at risk of transient cerebral ischemic attack because the frequency of transient cerebral ischemic attack is increased in a population of subjects who have undergone heart surgery as compared to a population of subjects who have not.

Other common risk factors for stroke include age, family history, gender, prior incidence of stroke, transient ischemic attack or heart attack, high blood pressure, smoking, diabetes, carotid or other artery disease, atrial fibrillation, other heart diseases such as heart disease, heart failure, dilated cardiomyopathy, heart valve disease and/or congenital heart defects; high blood cholesterol, and diets high in saturated fat, trans fat or cholesterol.

In prophylaxis, an active agent or procedure is administered to a subject at risk of a disease but not yet having the disease in an amount, frequency and route sufficient to prevent, delay or inhibit development of at least one sign or symptom of the disease. A prophylactically effective amount before administration or plasma level after administration means an amount or level of agent sufficient significantly to prevent, inhibit or delay at least one sign or symptom of the disease in a population of subjects (or animal models) at risk of the disease relative treated with the agent compared to a control population of subjects (or animal models) at risk of the disease not treated with an active agent of the invention. The amount or level is also considered prophylactically effective if an individual treated subject achieves an outcome more favorable than the mean outcome in a control population of comparable subjects not treated by methods of the invention. A prophylactically effective regime involves the administration of a prophylactically effective dose at a frequency and route of administration needed to achieve the intended purpose. For prophylaxis of stroke in a subject at imminent risk of stroke (e.g., a subject undergoing heart surgery), a single dose of agent is usually sufficient.

Depending on the agent, administration can be parenteral, intravenous, intrapulmonary, nasal, oral, subcutaneous, intra-arterial, intracranial, intrathecal, intraperitoneal, topical, intranasal or intramuscular.

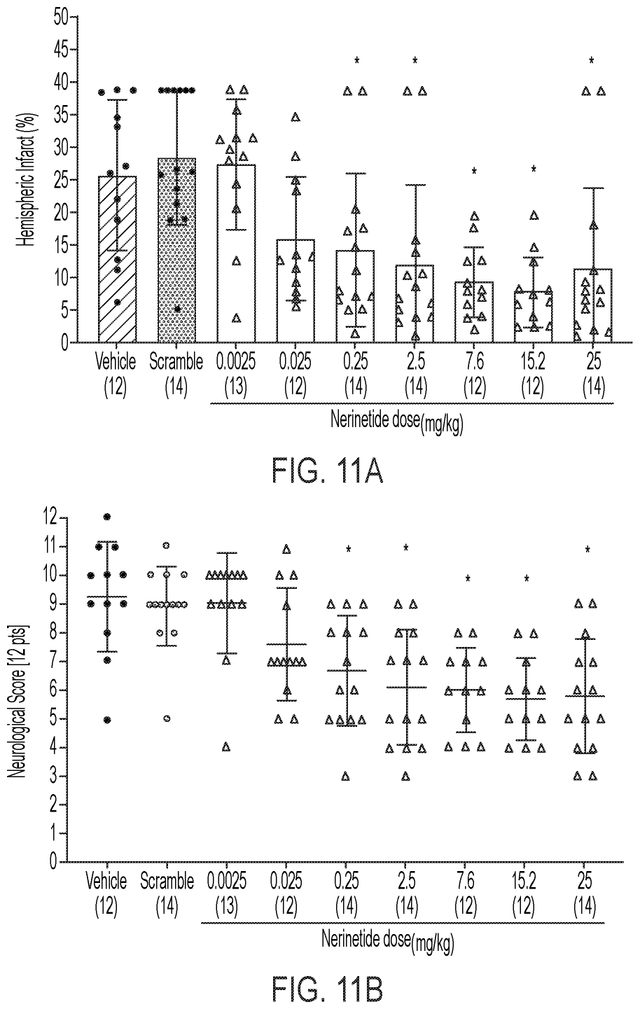

For intravenous administration, the claimed agents can be administered without anti-inflammatory e.g., up to 3 mg/kg, 0.1-3 mg/kg, 2-3 mg/kg or 2.6 mg/kg, or at higher dosages, e.g., at least 5, 10, 15, 20 or 25 mg/kg with an anti-inflammatory (see FIGS. 11A, B showing efficacy over a range of at least 0.25 mg/kg to 25 mg/kg). For routes such as subcutaneous, intranasal, intrapulmonary or intramuscular, the dose can be up to 10, 15, 20 or 25 mg/kg with or without an anti-inflammatory. The need for an-inflammatory at higher doses can alternatively be reduced or eliminated by administration of the active agent over a longer time period (e.g., administration in less than 1 minute, 1-10 minutes, and greater than ten minutes constitute alternative regimes in which for constant dosage histamine release and need for an anti-inflammatory is reduced or eliminated with increased time period).

The active agents can be administered as a single dose or as a multi-dose regime. A single dose regime can be used for treatment of an acute condition, such as acute ischemic stroke, to reduce infarction and cognitive deficits. Such a dose can be administered before onset of the condition if the timing of the condition is predictable such as with a subject undergoing neurovascular surgery, or within a window after the condition has developed (e.g., up to 1, 3, 6 or 12 hours later).

A multi-dose regime can be designed to maintain the active agent at a detectable level in the plasma over a prolonged period of time, such as at least 1, 3, 5 or 10 days, or at least a month, three months, six months or indefinitely. For example, the active agents can be administered every hour, 2, 3, 4, 6, or 12 times per day, daily, every other day, weekly and so forth. Such a regime can reduce initial deficits from an acute condition as for single dose administration and thereafter promote recovery from such deficits as still develop. Such a regime can also be used for treating chronic conditions, such as Alzheimer's and Parkinson's disease. Active agents are sometimes incorporated into a controlled release formulation for use in a multi-dose regime. Alternatively, multiple smaller doses could be administered over a shorter period to achieve neuroprotection without triggering histamine release, or given as a slow infusion if administered intravenously.

Active agents can be prepared with carriers that protect the compound against rapid elimination from the body, such as controlled formulations or coatings. Such carriers (also known as modified, delayed, extended or sustained release or gastric retention dosage forms, such as the DEPOMED GR™ system in which agents are encapsulated by polymers that swell in the stomach and are retained for about eight hours, sufficient for daily dosing of many drugs). Controlled release systems include microencapsulated delivery systems, implants and biodegradable, biocompatible polymers such as collagen, ethylene vinyl acetate, polyanhydrides, polyglycolic acid, polyorthoesters, polylactic acid, matrix controlled release devices, osmotic controlled release devices, multiparticulate controlled release devices, ion-exchange resins, enteric coatings, multilayered coatings, microspheres, nanoparticles, liposomes, and combinations thereof. The release rate of an active agent can also be modified by varying the particle size of the active agent: Examples of modified release include, e.g., those described in U.S. Pat. Nos. 3,845,770; 3,916,899; 3,536,809; 3,598,123; 4,008,719; 5,674,533; 5,059,595; 5,591,767; 5, 120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6, 113,943; 6, 197,350; 6,248,363; 6,264,970; 6,267,981; 6,376,461; 6,419,961; 6,589,548; 6,613,358; and 6,699,500.

VIII. Co-Administration with Anti-Inflammatories

Depending on the dose and route of administration the active agents of the invention can induce an inflammatory response characterized by mast cell degranulation and release of histamine and its sequelae. For example, dosages of at least 3 mg/kg are associated with histamine release for IV administration, and at least 10 mg/kg for other routes.

A wide variety of anti-inflammatory agents are readily available to inhibit one or more aspects of the of the inflammatory response. A preferred class of anti-inflammatory agent is mast cell degranulation inhibitors. This class of compounds includes cromolyn (5,5′-(2-hydroxypropane-1,3-diyl)bis(oxy)bis(4-oxo-4H-chromene-2-carboxylic acid) (also known as cromoglycate), and 2-carboxylatochromon-5′-yl-2-hydroxypropane derivatives such as bis(acetoxymethyl), disodium cromoglycate, nedocromil (9-ethyl-4,6-dioxo-10-propyl-6,9-dihydro-4H-pyrano[3,2-g]quinoline-2,8-di-carboxylic acid) and tranilast (2-{[(2E)-3-(3,4-dimethoxyphenyl)prop-2-enoyl]amino}), and lodoxamide (2-[2-chloro-5-cyano-3-(oxaloamino)anilino]-2-oxoacetic acid). Reference to a specific compound includes pharmaceutically acceptable salts of the compound Cromolyn is readily available in formulations for nasal, oral, inhaled or intravenous administration. Although practice of the invention is not dependent on an understanding of mechanism, it is believed that these agents act at an early stage of inflammatory response induced by an internalization peptide and are thus most effective at inhibiting development of its sequelae including a transient reduction in blood pressure. Other classes of anti-inflammatory agent discussed below serve to inhibit one or more downstream events resulting from mast cell degranulation, such as inhibiting histamine from binding to an H1 or H2 receptor, but may not inhibit all sequelae of mast cell degranulation or may require higher dosages or use in combinations to do so. Table 4 below summarizes the names, chemical formulate and FDA status of several mast cell degranulation inhibitors that can be used with the invention.

TABLE 4

| Drug Name | Alternative Names | Chemical Formula | FDA status |
|---|---|---|---|
| Azelastine | Astelin, Optivar | 4-[(4-chlorophenyl) | Approved |
| | | methyl]-2- | |
| | | (1-methylazepan-4- | |
| | | yl)phthalazin-1-one | |
| Bepotastine | Bepotastine | 4-[4-[(4- | Approved |
| | besilate, | chlorophenyl)- | |
| | Betotastine | pyridin-2- | |
| | besilate, | ylmethoxy] | |
| | TAU-284DS, | piperidin-1- | |
| | bepotastine | yl]butanoic acid | |
| Chlorzoxazone | Biomioran, EZE- | 5-chloro-3H-1,3- | Approved |
| | DS, Escoflex, | benzoxazol-2- | |
| | Flexazone, Mioran, | one | |
| | Miotran, | | |
| | Myoflexin, | | |
| | Myoflexine, | | |
| | Neoflex, | | |
| | Paraflex, Parafon | | |
| | Forte Dsc, | | |
| | Pathorysin, | | |
| | Relaxazone, | | |
| | Remular, | | |
| | Remular-S, | | |
| | Solaxin, | | |
| | Strifon Forte | | |
| | Dsc, Usaf Ma-10 | | |
| Cromolyn | Cromoglycate, | 5-[3-(2-carboxy-4- | Approved |
| | Chromoglicate, | oxochromen-6- | |
| | Chromoglicic | yl)oxy-2- | |
| | Acid, Aarane, | hydroxypropoxy]- | |
| | Alercom, Alerion, | 4- | |
| | Allergocrom, | oxochromene- | |
| | ApoCromolyn, | 2-carboxylic | |
| | Children't | acid | |
| | Nasalcrom, | | |
| | Colimune, Crolom, | | |
| | Cromolyn | | |
| | Nasal Solution, | | |
| | Cromoptic, | | |
| | Cromovet, Fivent, | | |
| | Gastrocrom, | | |
| | Gastrofrenal, | | |
| | GenCromoglycate, | | |
| | Inostral, Intal, | | |
| | Intal, Inhaler, | | |
| | Intal, Syncroner, | | |
| | Introl, Irtan, | | |
| | Lomudal, | | |
| | Lomupren, | | |
| | Lomusol, | | |
| | Lomuspray, | | |
| | Nalcrom, | | |
| | Nalcron, | | |
| | Nasalcrom, Nasmil, | | |
| | Opticrom, | | |
| | Opticron, | | |
| | Rynacrom, | | |
| | Sofro, Vistacrom, | | |
| | Vividrin | | |
| Epinastine | Elestat | C16H15N3, CAS | Approved |
| | | 80012-43-7 | |
| Isoproterenol | Aerolone, | 4-[1-hydroxy-2- | Approved |
| | Aleudrin, | (propan-2- | |
| | Aleudrine, | ylamino)ethyl] | |
| | Aludrin, | benzene-1,2- | |
| | Aludrine, | diol | |
| | Asiprenol, | | |
| | Asmalar, | | |
| | Assiprenol, | | |
| | Bellasthman, | | |
| | Bronkephrine, | | |
| | Euspiran, Isadrine, | | |
| | Isonorene, | | |
| | Isonorin, Isorenin, | | |
| | Isuprel, Isuprel | | |
| | Mistometer, | | |
| | Isupren, | | |
| | Medihaler-Iso, | | |
| | NeoEpinine, | | |
| | Neodrenal, | | |
| | Norisodrine, m | | |
| | Norisodrine, | | |
| | Aerotrol, | | |
| | Novodrin, | | |
| | Proternol, | | |
| | Respifral, | | |
| | Saventrine, | | |
| | Vapo-Iso | | |
| Ketotifen | Zaditor | C19H19NOS, | Approved |
| | | CAS 34580-14-8 | |
| Lodoxamide | Alomide | N,N′-(2-chloro- | Approved |
| (lodoxamide | | 5-cyano-m- | |
| tromethamine) | | phenylene) | |
| | | dioxamic acid | |
| | | tromethamine salt | |
| Nedocromil | Alocril, | 9-ethyl-4,6- | Approved |
| | Nedocromil | dioxo-10- | |
| | [USAN:BAN: | propylpyrano | |
| | INN],Tilade | [5,6-g]quinoline- | |
| | | 2,8-dicarboxylic | |
| | | acid | |
| Olopatadine | Olopatadine | 2-[(11Z)-11-(3- | Approved |
| | Hydrochloride | dimethylamino- | |
| | Patanol | propylidene)- | |
| | | 6H-benzo[c][2] | |
| | | benzoxepin-2- | |
| | | yl]acetic acid | |
| Pemirolast | Alamast | 9-methyl-3- | Approved |
| | | (2H-tetrazol-5- | |
| | | yl)pyrido[2,1-b] | |
| | | pyrimidin-4-one | |
| Pirbuterol | Maxair | 6-[2-(tert- | Approved |
| | | butylamino)-1- | |
| | | hydroxyethyl]-2- | |
| | | (hydroxymethyl) | |
| | | pyridin-3-ol | |

Another class of anti-inflammatory agent is anti-histamine compounds. Such agents inhibit the interaction of histamine with its receptors thereby inhibiting the resulting sequelae of inflammation noted above. Many anti-histamines are commercially available, some over the counter. Examples of anti-histamines are azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazines or terfenadine. Anti-histamines vary in their capacity to block anti-histamine in the CNS as well as peripheral receptors, with second and third generation anti-histamines having selectivity for peripheral receptors. Acrivastine, Astemizole, Cetirizine, Loratadine, Mizolastine, Levocetirizine, Desloratadine, and Fexofenadine are examples of second and third generation anti-histamines. Anti-histamines are widely available in oral and topical formulations. Some other anti-histamines that can be used are summarized in Table 5 below.

TABLE 5

| Drug | Alternative | Chemical | FDA |
|---|---|---|---|
| Name | Names | Formula | status |
| Ketotifen | Ketotifen, | C19H19NOS | Approved |
| fumarate | Zaditor | | |
| Mequitazine | Butix, | 10-(1- | Approved |
| | Instotal, | azabicyclo | |
| | Kitazemin, | [2.2.2]octan- | |
| | Metaplexan, | 8-ylmethyl) | |
| | Mircol, Primalan, | phenothiazine | |
| | Vigigan, | | |
| | Virginan, | | |
| | Zesulan | | |
| Dexbrom- | Ilvan | (3S)-3-(4- | Approved |
| pheniramine | | bromophenyl)- | |
| | | N,N- | |
| | | dimethyl-3- | |
| | | pyridin-2- | |
| | | ylpropan- | |
| | | 1-amine | |
| Methdilazine | Bristaline, | 10-[(1-methyl- | Approved |
| | Dilosyn, | pyrrolidin-3- | |
| | Disyncram, | yl)methyl] | |
| | Disyncran, | phenothiazine | |
| | Tacaryl, Tacaryl | | |
| | hydrochloride, | | |
| | Tacazyl, Tacryl | | |
| Chlor- | Aller-Chlor, | 3-(4-chloro- | Approved |
| pheniramine | Allergican, | phenyl)-N,N- | |
| | Allergisan, | dimethyl-3- | |
| | Antagonate, | pyridin-2- | |
| | Chlo- | ylpropan- | |
| | Amine, Chlor- | 1-amine | |
| | Trimeton, Chlor- | | |
| | Trimeton | | |
| | Allergy, | | |
| | Chlor-Trimeton | | |
| | Repetabs, Chlor- | | |
| | Tripolon, | | |
| | Chlorate, | | |
| | Chloropiril, | | |
| | Cloropiril, | | |
| | Efidac 24 | | |
| | Chlorpheniramine | | |
| | Maleate, Gen- | | |
| | Allerate, Haynon, | | |
| | Histadur, | | |
| | Kloromin, | | |
| | Mylaramine, | | |
| | Novo-Pheniram, | | |
| | Pediacare Allergy | | |
| | Formula, | | |
| | Phenetron, | | |
| | Piriton, | | |
| | Polaramine, | | |
| | Polaronil, | | |
| | Pyridamal | | |
| | 100, Telachlor, | | |
| | Teldrin | | |
| Bromo- | Bromfed, | 3-(4-bromo- | Approved |
| pheniramine | Bromfenex, | phenyl)-N,N- | |
| | Dimetane, | dimethyl-3- | |
| | Veltane | pyridin-2- | |
| | | ylpropan- | |
| | | 1-amine | |
| Terbutaline | Brethaire, | 5-[2-(tert- | Approved |
| | Brethine, Brican, | butylamino)-1- | |
| | Bricanyl, Bricar, | hydroxyethyl] | |
| | Bricaril, Bricyn | benzene-1,3-diol | |
| pimecrolimus | Elidel | (3S,4R,5S,8R, | Approved |
| | | 9E,12S,14S,15R, | as topical, |
| | | 16S,18R,19R, | Investi- |
| | | 26aS)-3-{(E)-2- | gational |
| | | [(1R,3R,4S)- | as oral |
| | | 4-Chloro-3- | |
| | | methoxy- | |
| | | cyclohexyl]-1- | |
| | | methylvinyl}- | |
| | | 8-ethyl- | |
| | | 5,6,8,11,12, | |
| | | 13,14,15,16,17, | |
| | | 18,19,24,25,26,26a- | |
| | | hexadecahydro-5, | |
| | | 19-dihydroxy-14, | |
| | | 16-dimethoxy- | |
| | | 4,10,12,18- | |
| | | tetramethyl- | |
| | | 15,19-epoxy- | |
| | | 3H-pyrido[2,1- | |
| | | c][1,4]oxaaza- | |
| | | cyclotricosine- | |
| | | 1,7,20,21 | |
| | | (4H,23H)-tetrone | |

Another class of anti-inflammatory agent useful in inhibiting the inflammatory response is corticosteroids. These compounds are transcriptional regulators and are powerful inhibitors of the inflammatory symptoms set in motion by release of histamine and other compounds resulting from mast cell degranulation. Examples of corticosteroids are Cortisone, Hydrocortisone (Cortef), Prednisone (Deltasone, Meticorten, Orasone), Prednisolone (Delta-Cortef, Pediapred, Prelone), Triamcinolone (Aristocort, Kenacort), Methylprednisolone (Medrol), Dexamethasone (Decadron, Dexone, Hexadrol), and Betamethasone (Celestone). Corticosteriods are widely available in oral, intravenous and topical formulations.

Nonsteroidal anti-inflammatory drugs (NSAIDs) can also be used. Such drugs include aspirin compounds (acetylsalicylates), non-aspirin salicylates, diclofenac, diflunisal, etodolac, fenoprofen, flurbiprofen, ibuprofen, indomethacin, ketoprofen, meclofenamate, naproxen, naproxen sodium, phenylbutazone, sulindac, and tometin. However, the anti-inflammatory effects of such drugs are less effective than those of anti-histamines or corticosteroids. Stronger anti-inflammatory drugs such as azathioprine, cyclophosphamide, leukeran, and cyclosporine can also be used but are not preferred because they are slower acting and/or associated with side effects. Biologic anti-inflammatory agents, such as Tysabri® or Humira®, can also be used but are not preferred for the same reasons.

Different classes of drugs can be used in combinations in inhibiting an inflammatory response. A preferred combination is a mast cell degranulation inhibitor and an anti-histamine.

In methods in which a PSD-95 inhibitor linked to an internalization peptide is administered with an anti-inflammatory agent, the two entities are administered sufficiently proximal in time that the anti-inflammatory agent can inhibit an inflammatory response inducible by the internalization peptide. The anti-inflammatory agent can be administered before, at the same time as or after the active agent. The preferred time depends in part on the pharmacokinetics and pharmacodynamics of the anti-inflammatory agent. The anti-inflammatory agent can be administered at an interval before the active agent such that the anti-inflammatory agent is near maximum serum concentration at the time the active agent is administered. Typically, the anti-inflammatory agent is administered between 6 hours before the active agent and one hour after. For example, the anti-inflammatory agent can be administered between 1 hour before and 30 min after the active agent. Preferably the anti-inflammatory agent is administered between 30 minutes before and 15 minutes after the active agent, and more preferably within 15 minutes before and the same time as the active agent. In some methods, the anti-inflammatory agent is administered before the active agent within a period of 15, 10 or 5 minutes before the active agent is administered. In some methods, the anti-inflammatory agent is administered 1-15, 1-10 or 1-5 minutes before the active agent.

When an anti-inflammatory agent is said to be able to inhibit the inflammatory response of an inhibitor peptide linked to an internalization peptide what is meant is that the two are administered sufficiently proximate in time that the anti-inflammatory agent would inhibit an inflammatory response inducible by the inhibitor peptide linked to the internalization peptide if such a response occurs in a particular subject, and does not necessarily imply that such a response occurs in that subject. Some subjects are treated with a dose of an inhibitor peptide linked to an internalization peptide that is associated with an inflammatory response in a statistically significant number of subjects in a controlled clinical or nonclinical trial. It can reasonably be assumed that a significant proportion of such subjects although not necessarily all develop an anti-inflammatory response to the internalization peptide linked to the internalization peptide. In some subjects, signs or symptoms of an inflammatory response to the inhibitor peptide linked to the internalization peptide are detected or detectable.

In clinical treatment of an individual subject, it is not usually possible to compare the inflammatory response from an inhibitor peptide linked to an internalization peptide in the presence and absence of an anti-inflammatory agent. However, it can reasonably be concluded that the anti-inflammatory agent inhibits an anti-inflammatory response inducible by the peptide if significant inhibition is seen under the same or similar conditions of co-administration in a controlled clinical or pre-clinical trial. The results in the subject (e.g., blood pressure, heart rate, hives) can also be compared with the typical results of a control group in a clinical trial as an indicator of whether inhibition occurred in the individual subject. Usually, the anti-inflammatory agent is present at a detectable serum concentration at some point within the time period of one hour after administration of the pharmacologic agent. The pharmacokinetics of many anti-inflammatory agents is widely known and the relative timing of administration of the anti-inflammatory agent can be adjusted accordingly. The anti-inflammatory agent is usually administered peripherally, i.e., segregated by the blood brain barrier from the brain. For example, the anti-inflammatory agent can be administered orally, nasally, intravenously or topically depending on the agent in question. If the anti-inflammatory agent is administered at the same time as the pharmacologic agent, the two can be administered as a combined formulation or separately.

In some methods, the anti-inflammatory agent is one that does not cross the blood brain barrier when administered orally or intravenously at least in sufficient amounts to exert a detectable pharmacological activity in the brain. Such an agent can inhibit mast cell degranulation and its sequelae resulting from administration of the active agent in the periphery without itself exerting any detectable therapeutic effects in the brain. In some methods, the anti-inflammatory agent is administered without any co-treatment to increase permeability of the blood brain barrier or to derivatize or formulate the anti-inflammatory agent so as to increase its ability to cross the blood brain barrier. However, in other methods, the anti-inflammatory agent, by its nature, derivatization, formulation or route of administration, may by entering the brain or otherwise influencing inflammation in the brain, exert a dual effect in suppressing mast-cell degranulation and/or its sequelae in the periphery due to an internalization peptide and inhibiting inflammation in the brain. Strbian et al., WO 04/071531 have reported that a mast cell degranulation inhibitor, cromoglycate, administered i.c.v. but not intravenously has direct activity in inhibiting infarctions in an animal model.

In some methods, the subject is not also treated with the same anti-inflammatory agent co-administered with the active agent in the day, week or month preceding and/or following co-administration with active agent. In some methods, if the subject is otherwise being treated with the same anti-inflammatory agent co-administered with the active agent in a recurring regime (e.g., same amount, route of delivery, frequency of dosing, timing of day of dosing), the co-administration of the anti-inflammatory agent with the active agent does not comport with the recurring regime in any or all of amount, route of delivery, frequency of dosing or time of day of dosing. In some methods, the subject is not known to be suffering from an inflammatory disease or condition requiring administration of the anti-inflammatory agent co-administered with the active agent in the present methods. In some methods, the subject is not suffering from asthma or allergic disease treatable with a mast cell degranulation inhibitor. In some methods, the anti-inflammatory agent and active agent are each administered once and only once within a window as defined above, per episode of disease, an episode being a relatively short period in which symptoms of disease are present flanked by longer periods in which symptoms are absent or reduced.

The anti-inflammatory agent is administered in a regime of an amount, frequency and route effective to inhibit an inflammatory response to an internalization peptide under conditions in which such an inflammatory response is known to occur in the absence of the anti-inflammatory. An inflammatory response is inhibited if there is any reduction in signs or symptoms of inflammation as a result of the anti-inflammatory agent. Symptoms of the inflammatory response can include redness, rash such as hives, heat, swelling, pain, tingling sensation, itchiness, nausea, rash, dry mouth, numbness, airway congestion. The inflammatory response can also be monitored by measuring signs such as blood pressure, or heart rate. Alternatively, the inflammatory response can be assessed by measuring plasma concentration of histamine or other compounds released by mast cell degranulation. The presence of elevated levels of histamine or other compounds released by mast cell degranulation, reduced blood pressure, skin rash such as hives, or reduced heart rate are indicators of mass cell degranulation. As a practical matter, the doses, regimes and routes of administration of most of the anti-inflammatory agents discussed above are available in the Physicians' Desk Reference and/or from the manufacturers, and such anti-inflammatories can be used in the present methods consistent with such general guidance.

Although the invention has been described in detail for purposes of clarity of understanding, certain modifications may be practiced within the scope of the appended claims. All publications, accession numbers, and patent documents cited in this application are hereby incorporated by reference in their entirety for all purposes to the same extent as if each were so individually denoted. To the extent more than one sequence is associated with an accession number at different times, the sequences associated with the accession number as of the effective filing date of this application is meant. The effective filing date is the date of the earliest priority application disclosing the accession number in question. Unless otherwise apparent from the context any element, embodiment, step, feature or aspect of the invention can be performed in combination with any other.

### EXAMPLES

### Example 1

We sought to determine whether treatment with nerinetide, with or without usual care with intravenous alteplase, would improve outcomes for subjects with ischemic stroke due to large vessel occlusion with potentially salvageable brain determined by imaging criteria, in the setting of rapid reperfusion now attainable by endovascular thrombectomy (EVT).

### Methods

### Study Design

ESCAPE-NA1 was a multicenter, randomized, double-blinded, placebo-controlled, parallel group, single-dose study to determine the efficacy and safety of intravenous nerinetide in patients with acute ischemic stroke who were selected to undergo thrombectomy. Patients were randomized in a 1:1 ratio to receive a single, 2.6 mg/kg (up to a maximum dose of 270 mg) intravenous dose of nerinetide or saline placebo delivered over 10+1 minutes. Nerinetide and placebo were prepared as colorless solutions in numbered, refrigerated vials.

### Randomisation and Masking

Randomization in a 1:1 ratio to nerinetide or placebo occurred using a real-time, dynamic, Internet-based, stratified randomized minimization procedure. Stratification occurred on the use of intravenous alteplase (yes/no) and declared initial thrombectomy device (stent-retriever or aspiration device). The choice to stratify was based upon the possibility of drug-drug or drug-device interactions. Randomized minimization occurring within strata aimed to achieve distribution balance with regard to age, sex, baseline National Institutes of Health Stroke Scale (NIHSS) score (range, 0 to 42, with higher scores indicating greater stroke severity), site of arterial occlusion, baseline Alberta Stroke Program Early Computed Tomography Score (ASPECTS; range, 0 to 10, with 1 point subtracted for any evidence of early ischemic change in each defined region on the CT scan) and clinical site.

### Participants

Eligible patients were adults aged 18 or greater with a disabling ischemic stroke at the time of randomization (baseline NIHSS>5), who had been functioning independently in the community (Barthel Index score>90 [range, 0 to 100, with higher scores indicating a greater ability to complete activities of daily living])¹ before the stroke. Enrollment occurred up to 12 hours after the onset of stroke symptoms (last-seen-well time). Non-contrast CT and multiphase CTA were performed at the thrombectomy center to identify patients with a confirmed proximal intracranial artery occlusion, defined as the intracranial internal carotid artery or the first segment of the middle cerebral artery or both. Patients had a small-to-moderate ischemic core (defined as ASPECTS of 5 to 10, range: 0-10; Alberta Stroke Program Early CT Score; aspectsinstroke.com; lower score suggests greater extent of acute ischemic changes) and moderate-to-good collateral circulation (aspectsinstroke.com/collateral-scoring), defined as the filling of 50% or more of the middle-cerebral artery pial arterial circulation on CTA.

### Procedures

After qualifying imaging, patients were treated with rapid EVT using currently available devices. Some patients received intravenous alteplase according to usual care following national or regional guidelines, before or during EVT, at a primary hospital prior to transfer or at the endovascular center. Interpretation of treatment guidelines was at the discretion of the treating team. Patients treated with alteplase more than 4.5 hours from stroke onset were not excluded from the trial for this reason alone. Patients had to meet inclusion and exclusion criteria at the EVT hospital. Patients received trial drug, as a single dose of 2.6 mg/kg to a maximum dose of 270 mg, based upon estimated or actual weight (if known) using a dedicated intravenous line. The trial drug was administered as soon as possible after randomization.

Time targets were imaging to randomization 30 minutes, imaging to study drug administration ≤60 minutes, and imaging to arterial access/puncture ≤60 minutes. Targets from imaging to reperfusion were 90^{th} percentile ≤90 minutes and a median at ≤75 minutes. In general, the order of events was imaging to determine eligibility for EVT treatment, study randomization, administration of alteplase (in some patients), administration of nerinetide, and performance of EVT.

### Clinical Assessments and Outcomes

All patients had standard assessments of demographic characteristics, medical history, laboratory values and stroke severity (NIHSS score). In some patients, up to 6 consecutive blood samples were drawn following dosing for pharmacokinetic analysis of nerinetide levels.

The primary outcome was good outcome as defined by a score of 0-2 on the modified Rankin scale (mRS) (range, 0 [no symptoms] to 6 [death]) for the assessment of neurologic functional disability² assessed in person or, if an in-person visit was not possible, by telephone, at 90 days after randomization by personnel certified in the scoring of the mRS. Secondary efficacy outcomes were neurological outcome as defined by the NIHSS of 0-2, functional independence in activities of daily living as defined by a Barthel Index score of ≥95, excellent functional outcome as defined by a score of 0-1 on the mRS and mortality rates. Tertiary outcomes included assessments of stroke volumes on 24-hour imaging (MR or CT brain). Prespecified safety outcomes were all serious adverse events and mortality. Imaging interpretation was conducted at a central core laboratory and clinical data were verified by independent monitors. Infarct volumes were measured by summation of manual planimetric demarcation of infarct on axial imaging (636/1099 (57.9%) on CT and 463/1099 (42.1%) on MRI).

### Statistical Analysis

The trial was designed to have 80% power to detect an 8.7% absolute difference between the proportion of patients achieving a mRS 0-2 at 90 days post-randomization in the nerinetide and placebo groups. Because we used randomised minimization, a post-hoc permutation test was used with 5000 simulations and confirmed the integrity of the randomization process which produced covariate balance between treatment groups. The sample size used a 2-sided alpha level 0.05 and accounted for a single interim analysis when 600 patients completed their 90-day follow-up accounting for alpha spending using an O′Brien-Fleming boundary (Z=2.784, p=0.003).

The primary analysis was conducted on the intention-to-treat (ITT) population, and was an adjusted estimate of effect size including treatment and the stratification variables of intravenous alteplase and declared initial endovascular approach, and the baseline covariates of age, sex, baseline NIHSS score, baseline ASPECTS, occlusion location and clinical site. We report risk ratios derived using multivariable Poisson regression with the Huber-White robust variance estimator. This allows direct comparison with the unadjusted estimates of effect and provides a more intuitively understood representation of the treatment effect size. A hierarchical approach was used to control for multiple comparisons, starting with the primary outcome and proceeding to secondary outcomes in the following order: shift analysis of 90-day mRS under proportional odds model across the mRS scale, NIHSS 0-2 vs. 3 or greater at 90 days, BI at 95-100 vs. 0-90, mortality rate at 90 days, and the proportion of subjects with mRS score of 0-1 at day 90. All outcomes at and following the demonstration of no difference with a two-sided p>0.05 were considered exploratory and not adjusted for multiplicity. Exploratory analyses for heterogeneity of treatment effect, to evaluate drug-drug and drug-device interactions, were performed on the two stratification variables of alteplase use and declared initial endovascular device choice. Exploratory analyses on 11 additional sub-groups of interest identified a priori in the statistical analysis plan were performed. Infarct volumes showed a skewed distribution and were reported as the median and interquartile range; infarct volumes by treatment group were compared using a t-test on cubic root transformed volumes. A Cox proportional hazards model provided an adjusted hazard ratio of the relative time to death by treatment assignment.

Analyses were conducted on the intent-to-treat (ITT) population, defined as all patients randomized into the trial, regardless of treatment received. Deceased patients were included in the ITT population with a mRS score of 6, a Barthel Index of 0 and NIHSS of 42. Missing primary outcomes (n=9) were imputed as the worst possible score, counted as poor outcome (mRS 3-6 dichotomy) and for mortality analysis, imputed as deaths. All analyses were performed with the use of SAS software, (v9.4, SAS Institute) or STATA (v16.0).

### Findings

### Patients

Between Mar. 1, 2017 and Aug. 12, 2019, 1105 patients were enrolled with 549 assigned to receive nerinetide and 556 to receive placebo. Primary outcome data were missing for 9 patients (0.81%; lost to follow-up: 2; withdrawal of consent: 7). These patients were considered non-responders. Baseline characteristics were similar in the two groups (Table 1).

Of 1105 enrolled patients, 4 (0.4%; 2 in each group) patients did not receive any study drug and 25 (2.3%; 14 placebo, 11 nerinetide) received the correct drug but incorrect volume or duration. There were no crossovers. All patients underwent attempted EVT; 8 did not have selective cerebral angiography completed; 1 withdrew consent prior to EVT. Usual care treatment with intravenous alteplase occurred in 330 (60.1%) in the nerinetide patients and 329 (59.2%) in the placebo patients. The declared first device was a stent retriever in 850 (76.9%) patients equally divided between nerinetide and placebo patients. The overall workflow (imaging to randomization, imaging to study drug, study drug to reperfusion) and quality of reperfusion (on the expanded Thrombolysis in Cerebral Ischemia (eTICI) scale) were similar in both arms (Table 1), with the exception of longer onset to treatment times in the no alteplase stratum. The onset of stroke to randomization time was 160-537 min (mean 275 min), 142-541 min (mean 270 min), 112-228 min (mean 161 min) and 109-240 min (mean 152 min) in the no alteplase placebo, no alteplase nerinetide, alteplase placebo and alteplase nerinetide strata respectively. In other words, the no alteplase strata were treated with nerinetide about two hours later post-onset of stroke than the alteplase strata. In a condition characterized by the adage time means brain, the no alteplase strata represents a much more difficult subset of patients to treat than the alteplase strata.

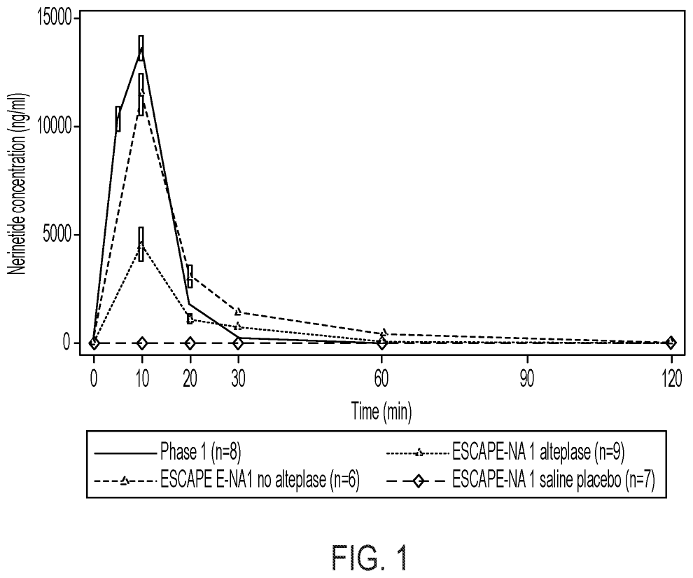

Nerinetide plasma levels were obtained from 22 subjects in ESCPAE-NA1 and previously acquired data from 8 healthy volunteer subjects receiving a single dose of 2.6 mg/kg nerinetide intravenously. Time 0 is a preinfusion time-point. Among ESCAPE-NA-1 patients who received alteplase there was a reduction in nerinetide plasma concentration compared to patients who did not receive alteplase and to historical non-stroke patients not receiving alteplase. The bars represent standard error of the mean. FIG. 1 shows in the absence of alteplase, nerinetide reached peak levels after ten minutes and declined to background by about 120 minutes. In the presence of alteplase, nerinetide's maximum level was reduced by more than 50% with decline to background level by 60 minutes. AUC was similarly reduced. (p=0.0119, mixed effects linear regression).

### Outcomes

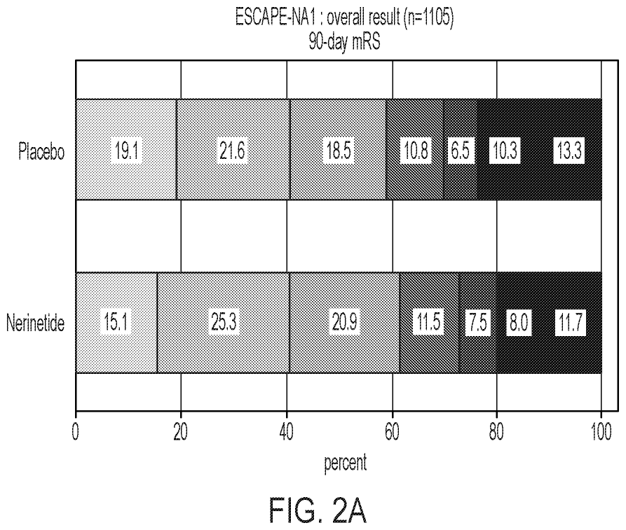

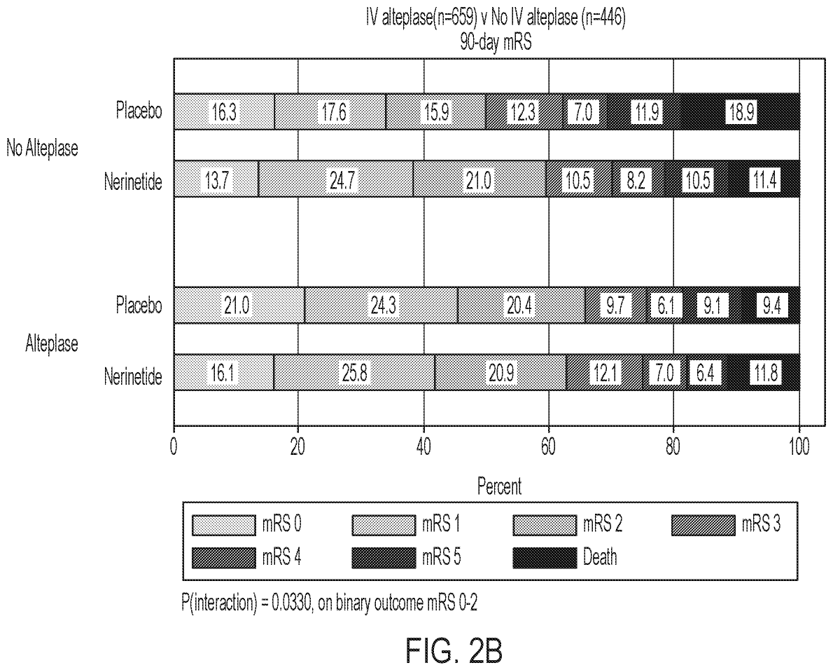

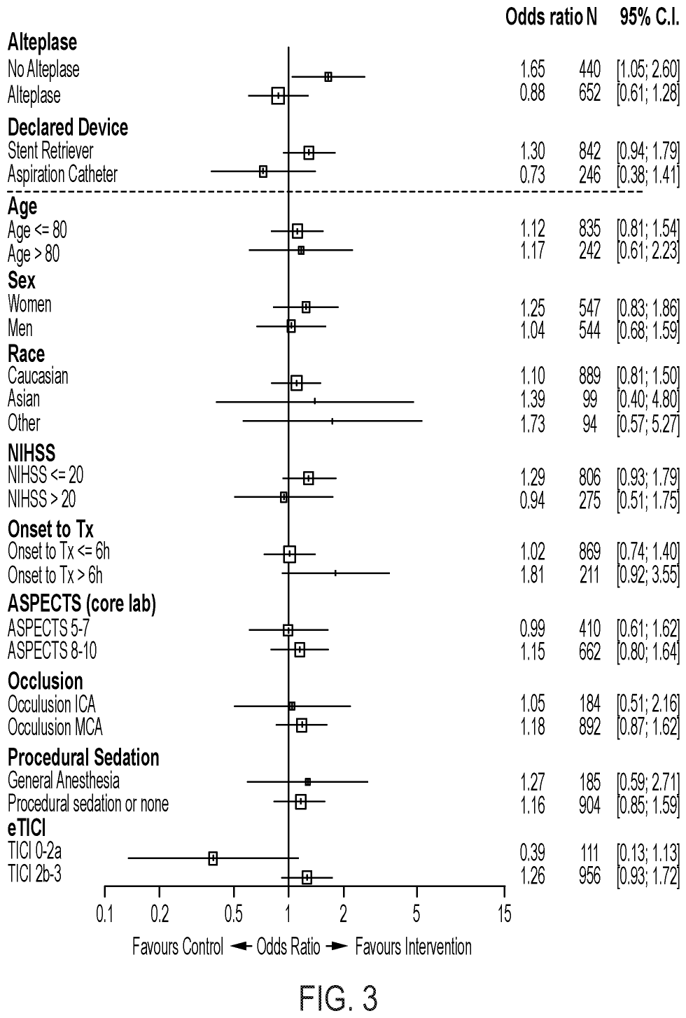

The primary outcome of the proportion of patients achieving a mRS 0-2 at 90 days was 61.4% in nerinetide and 59.2% in placebo (adj RR=1.04; CI₉₅ 0.96-1.14; p=0.350). Secondary outcomes are shown in Table 2A and exploratory subgroups in FIGS. 2A, B and FIG. 3.

Participant characteristics were well balanced within each of the device and alteplase strata except that subjects not receiving alteplase had longer average times from stroke onset to randomization. This was because subjects receiving alteplase were generally enrolled within the window dictated by alteplase treatment guidelines (treatment window of <4.5 hours from last known well), whereas those not receiving alteplase were enrolled over the full 12 hour enrollment window permitted by the protocol. There was no evidence of treatment effect modification by declared choice of first endovascular device. By contrast, there was evidence of treatment effect modification by usual care intravenous alteplase use (Table 2B, FIG. 2B).

In the stratum that did not receive alteplase, 59.3% of patients receiving nerinetide as compared to 49.8% receiving placebo achieved an mRS 0-2 (adj RR 1.18, CI₉₅ 1.01-1.38). There was a 7.5% absolute risk reduction in mortality at 90 days. This resulted in an approximate halving of the hazard of death (adj HR 0.56, CI₉₅ 0.35-0.95). In the stratum that received alteplase, the proportions of patients achieving an mRS 0-2 were similar (62.7% nerinetide vs. 65.7% placebo (adj RR 0.97, CI₉₅ 0.87-1.08). The observed treatment effect modification by alteplase is supported by reductions in peak plasma nerinetide levels in the alteplase stratum (FIG. 1). Other pre-specified exploratory sub-groups of interest showed no evidence of differential treatment effect (FIG. 3).

Median infarct volumes in the nerinetide group were 26.0 (iqr 6.6-101.5) ml and 23.7 (iqr 6.4-78.9) ml in the placebo group. There were no differences in infarct volume between nerinetide and placebo groups by declared endovascular device strata. In the alteplase stratum, there was no difference in median infarct volume (21.1 vs. 22.7 ml) between treatment groups. In the no alteplase stratum, there was a reduction in median infarct volumes in the nerinetide group (39.2 vs. 26.7 ml) (Table 2B).

### Safety

The safety population included all patients who received any amount of study drug (n=1101). There were no differences in important safety outcomes. (Table 3).

### Interpretation

In the no alteplase stratum, nerinetide was associated with improved outcomes, and in the alteplase stratum there was no observed benefit with the absolute risk difference slightly (non-significantly) favoring placebo.

The observation of effect modification by alteplase on nerinetide was unexpected. Available data from pre-clinical animal studies suggested that when nerinetide was administered after alteplase, the treatment effect of nerinetide was preserved. The large magnitude of the effect of alteplase on nerinetide treatment response in humans was not predicted. The finding can be explained by drug-drug interaction between alteplase and nerinetide nullifying the treatment effect of nerinetide in alteplase stratum and a 9.4% absolute benefit (Number-needed-to-treat of 10-11 patients) in the no-alteplase stratum. This lack of effectiveness of nerinetide in the alteplase stratum is biologically plausible. Nerinetide does not affect the activity of alteplase³. However, nerinetide has amino-acid sequences cleaved by plasmin, a serine protease generated from circulating plasminogen by tissue-plasminogen activators (such as alteplase) and is cleaved by alteplase in animals. The lack of benefit of nerinetide in the alteplase stratum is likely due to enzymatic cleavage of nerinetide by plasmin leading to subtherapeutic concentrations of nerinetide, as supported by the pharmacokinetic data from a subset of trial participants. Because cleavage of nerinetide is an indirect effect of alteplase, the duration of time between alteplase infusion and nerinetide administration may be less important as compared with the duration of activity and ongoing generation of plasmin. The improvement in clinical outcomes, reduction in mortality and reduction in infarct volumes in the no alteplase stratum combined with the pharmacokinetic observations provide compelling evidence that the clinical observation of effect modification is not a chance finding.

Patients in the alteplase stratum were generally enrolled within the therapeutic window of alteplase (up to 4.5 hours from stroke onset), whereas those in the no-alteplase stratum were enrolled throughout the 12-hour stroke onset-to-randomization window of the trial. In general, there was collinearity between the use of alteplase with time; the no-alteplase stratum was much more likely to include patients with longer onset-to-randomization time.

Equal numbers of serious adverse events occurred in both nerinetide and placebo groups. At high doses in animals, nerinetide causes a transient elevation of circulating histamine thought to be due to a non-immune mediated mast-cell degranulation similar to that caused by highly charged cationic molecules like protamine and vancomycin. This could cause adverse histamine-triggered reactions such as hypotension, flushing, urticaria, and pruritis. There were no significant differences in rates of adverse events in patients treated with nerinetide compared with placebo. However, there were numerically more instances of transient hypotension, pneumonia and congestive cardiac failure with the drug compared to placebo. Among the no alteplase stratum, the nerinetide group had numerically less than instances of stroke progression, recurrent stroke, and hemorrhagic transformation compared to placebo.

TABLE 1
Baseline Characteristics

| | | Placebo | Nerinetide |
|---|---|---|---|
| | | (N = 556) | (N = 549) |
| Demographics | | | |
| Age (y) | | 70.3 | 71.5 |
| | | (60.4-80.1) | (61.1-79.7) |
| Sex Female (%) | | 281 (50.5%) | 268 (48.8%) |
| Race* | Caucasian | 453 (81.5%) | 436 (79.4%) |
| | Asian | 52 (9.4%) | 55 (10.0%) |
| Medical History* | | | |
| Hypertension | | 396 (71.4%) | 378 (68.9%) |
| Non-smoker (lifelong) | | 280 (50.7%) | 285 (52.1%) |
| Hyperlipidemia | | 260 (46.9%) | 254 (46.3%) |
| Atrial fibrillation | | 192 (34.6%) | 195 (35.5%) |
| Ischaemic heart disease | | 130 (23.4%) | 122 (22.3%) |
| Diabetes | | 107 (19.3%) | 111 (20.2%) |
| Congestive heart failure | | 65 (11.7%) | 72 (13.1%) |
| Any past stroke | | 76 (13.7%) | 81 (14.8%) |
| Peripheral vascular disease | | 28 (5.1%) | 31 (5.7%) |
| Chronic renal failure | | 28 (5.1%) | 35 (6.4%) |
| Recent major surgery | | 21 (3.8%) | 18 (3.3%) |
| Clinical Factors | | | |
| Witnessed stroke onset§ | | 309 (55.9%) | 319 (58.2%)* |
| Stroke-on-awakening§ | | 84 (15.1%) | 92 (16.8%) |
| Right hemisphere stroke | | 301 (54.2%) | 280 (51.0%) |
| NIHSS | | 17 | 17 |
| | | (13-21) | (12-21) |
| Systolic blood pressure (mm Hg) | | 146.6 | 146 |
| | | (130-163) | (131-165) |
| Glucose (mM) | | 6.7 | 6.7 |
| | | (5.9-7.8) | (5.9-8.0) |
| ECG showing atrial | | 133 (25.4%) | 131 (25.7%) |
| fibrillation at baseline | | | |
| ASPECTS (core lab determined)** | | 8 | 8 |
| | | (7-9) | (7-9) |
| ASPECTS 8-10 (site determined at | | 403 (72.5%) | 397 (72.3%) |
| randomization) | | | |
| Occlusion site ICA (site determined at | | 103 (18.5%) | 110 (20.0%) |
| randomization) | | | |
| Collaterals-good (site determined at | | 344 (62.6%) | 355 (65.4%) |
| randomization) | | | |
| Treatment & Workflow | | | |
| Alteplase treatment | | 329 (59.2%) | 330 (60.1%) |
| Interhospital transfer to EVT hospital | | 235 (42.3%) | 228 (41.5%) |
| General anesthesia use | | 97 (17.5%) | 95 (17.4%) |
| Onset-to-randomization time (min) | | 188 | 186 |
| | | (122-311) | (120-309) |
| Door-to-arterial access/puncture (min) | | 58 | 60 |
| | | (42-83) | (41.5-84) |
| Study drug start-to-reperfusion (min) | | 23 | 21 |
| | | (8-42) | (8-40) |
| eTICI (core lab determined) | 2b/2c/3 | 480 (87.0%) | 476 (87.2%) |
| | 2c/3 | 259 (46.9%) | 247 (45.2%) |

*N = 546 (3 with missing data);
**N = 1090 due to missing or unscoreable imaging;
§. In the situation where the stroke was not witnessed, stroke onset was defined as the last seen well time. This often meant the time the patient went to bed in the case of stroke on awakening.
All values displayed as median (iqr) or n (%)
NIHSS = National Institutes of Health Stroke Scale;
ECG = electrocardiogram;
ASPECTS = Alberta Stroke Program Early CT Score;
ICA = internal carotid artery;
EVT = endovascular thrombectomy;
eTICI = expanded Thrombolysis In Cerebral Ischemia

TABLE 2A
Overall Outcomes
Adjusted Outcomes (Pre-specified Primary Analysis)

| Primary Outcome | Risk ratio (95% confidence interval) | | |
|---|---|---|---|
| mRS 0-2 | 1.04 (0.96-1.14) | | |
| Secondary Outcomes | | | |
| NIHSS 0-2 | 1.01 (0.92-1.11) | | |
| BI 95-100 | 1.03 (0.94-1.12) | | |
| Mortality | 0.84 (0.63-1.13) | | |
| mRS 0-1 | 0.98 (0.85-1.12) | | |
| Infarct volume | −0.29 (−0.87 to 0.30)** | | |
| (cubic root | | | |
| transformation; | | | |
| mean, ml^{1/3})** | | | |
| Unadjusted Effect size | Placebo | Nerinetide | |
| Primary Outcome | (n = 556) | (n = 549) | RR (CI₉₅) |
| mRS 0-2 | 329 (59.2%) | 337 (61.4%) | 1.04 (0.94-1.14) |
| Secondary Outcomes | | | |
| NIHSS 0-2 | 320 (57.6%) | 320 (58.3%) | 1.01 (0.92-1.12) |
| BI 95-100 | 335 (60.3%) | 341 (62.1%) | 1.03 (0.94-1.13) |
| Mortality§ | 80 (14.4%) | 67 (12.2%) | 0.85 (0.63-1.15) |
| mRS 0-1 | 226 (40.6%) | 222 (40.4%) | 0.99 (0.86-1.15) |
| Infarct volume | 26.0 | 23.7 | −2.3* |
| (median, iqr; ml) | (6.6-101.5) | (6.4-78.9) | |

*Absolute volume difference of medians.
**The beta coefficient represents the adjusted reduction in cubic root volume (ml^{1/3}) with nerinetide (NA-1) compared to control. N = 1099 due to missing or unmeasurable volumes on imaging. The mean volumes were 73.1 ml (placebo) and 71.1 ml (nerinetide).
§Without imputation of 9 patients with missing outcomes to death, there are 74/550 (13.5%) deaths in the placebo group and 64/546 (11.7%) deaths in the nerinetide group; RR 0.87 (CI₉₅ 0.64-1.19)
mRS = modified Rankin Scale;
NIHSS = National Institutes of Health Stroke Scale;
BI = modified Barthel Index;
RR = risk ratio;
CI₉₅ = 95% confidence interval
Notes:
Risk ratios are derived using multivariable Poisson regression with the Huber-White robust variance estimator. This approach differs from our SAP (which stated that we would report odds ratios from a multivariable logistic regression) because it was recommended at the time of peer review by both a reviewer and by the editor. The proportional odds assumption was not satisfied (Score test) and therefore the common odds ratio for ‘shift’ across the modified Rankin Scale is not reported. Adjustment for: age (y), sex, baseline NIHSS score, ASPECTS score read by the core lab, occlusion location as MCA vs. ICA, declared endovascular approach and site.

TABLE 2B
Outcomes by Alteplase

| | No Alteplase | Alteplase |
|---|---|---|
| Adjusted Outcomes (Pre- | (n = 446) | (n = 659) |
| specified Primary Analysis) | Risk ratio (95% confidence interval) | |
| Primary Outcome | | |
| mRS 0-2 | 1.18 (1.01-1.38) | 0.97 (0.87-1.08) |
| Secondary Outcomes | | |
| NIHSS 0-2 | 1.14 (0.97-1.34) | 0.92 (0.82-1.04) |
| BI 95-100 | 1.14 (0.97-1.34) | 0.97 (0.88-1.08) |
| Mortality | 0.66 (0.44-0.99) | 1.08 (0.70-1.66) |
| mRS 0-1 | 1.04 (0.82-1.31) | 0.91 (0.78-1.08) |
| Infarct volume (cubic | −0.98 (−1.91 to −0.05)** | 0.20 (−0.56 to 0.95)** |
| root transformation; | | |
| mean, ml^{1/3})** | | |

| Unadjusted Effect | Placebo | | Nerinetide | | RR | Placebo | Nerinetide | RR |
|---|---|---|---|---|---|---|---|---|
| size | (n = 227) | | (n = 219) | | (CI₉₅) | (n = 329) | (n = 330) | (CI₉₅) |
| Primary Outcome | | | | | | | | |
| mRS 0-2 | 113 | (49.8%) | 130 | (59.3%) | 1.19 (1.01-1.41) | 216 (65.7%) | 207 (62.7%) | 0.96 (0.85-1.07) |
| Secondary Outcomes | | | | | | | | |
| NIHSS 0-2 | 113 | (49.8%) | 129 | (58.9%) | 1.18 (1.00-1.40) | 207 (62.9%) | 191 (57.9%) | 0.92 (0.81-1.04) |
| BI 95-100 | 114 | (50.2%) | 128 | (58.4%) | 1.16 (0.98-1.38) | 221 (67.2%) | 213 (64.5%) | 0.96 (0.86-1.07) |
| Mortality§ | 46 | (20.3%) | 28 | (12.8%) | 0.63 (0.41-0.97) | 34 (10.3%) | 39 (11.8%) | 1.14 (0.74-1.76) |
| mRS 0-1 | 77 | (33.9%) | 84 | (38.4%) | 1.13 (0.88-1.45) | 149 (45.2%) | 138 (41.8%) | 0.92 (0.77-1.10) |
| Infarct volume | 39.2 | (9.2-132.9) | 26.7 | (6.3-88.0) | −12.5* | 21.1 | 22.7 | 1.6* |
| (median, ml) | | | | | | | | |

*Absolute volume difference of medians.
**The beta coefficient represents the reduction in cubic root volume (ml^{1/3}) with nerinetide (NA-1) compared to control. Effect modification of alteplase on nerinetide for the infarct volume outcome, p_{interaction} = 0.0400. In no alteplase group, the mean volumes were 87.2 ml (placebo) and 67.8 ml (nerinetide). In the alteplase group, the mean volumes were 63.3 ml (placebo) and 73.3 ml (nerinetide)
§Without imputation of 9 patients with missing outcomes to death: (1) No Alteplase stratum - there are 43/224 (19.2%) deaths in the placebo group and 25/216 (11.6%) deaths in the nerinetide group; RR 0.60 (CI₉₅ 0.38-0.95); (2) Alteplase stratum - there are 31/326 (9.5%) deaths in the placebo group and 39/330 (11.8%) deaths in the nerinetide group; RR 1.24 (CI₉₅ 0.80-1.94)
Notes:
Effect modification of alteplase on nerinetide for the mRS 0-2 outcome, p_{interaction} = 0.0330. Missing data for binary outcomes imputed with the worst possible score (No alteplase stratum, 3 in control, 3 in nerinetide; alteplase stratum, 3 in control, 0 in nerinetide). Risk ratios are derived using multivariable Poisson regression with the Huber-White robust variance estimator. This approach differs from our SAP (which stated that we would report odds ratios from a multivariable logistic regression) because it was recommended at the time of peer review by both a reviewer and by the editor. The proportional odds assumption was not satisfied (Score test) and therefore the common odds ratio for ‘shift’ across the modified Rankin Scale is not reported. Adjustment for: age (y), sex, baseline NIHSS score, ASPECTS score read by the core lab, occlusion location as MCA vs. ICA, declared endovascular approach and site.
mRS = modified Rankin Scale;
NIHSS = National Institutes of Health Stroke Scale;
BI = modified Barthel Index;
RR = risk ratio;
CI₉₅ = 95% confidence interval

TABLE 3
Treatment Emergent Serious Adverse Events by MedDRA Preferred Term

| | Placebo | Nerinetide | RR* |
|---|---|---|---|
| | (n = 554) | (n = 547) | (95% CI) |
| Any serious | 198 (35.7%) | 181 (33.1%) | 0.92 (0.79-1.09) |
| adverse Event | | | |
| Stroke-in-evolution | 43 (7.8%) | 36 (6.6%) | 0.85 (0.55-1.30) |
| (progression) | | | |
| Ischaemic stroke (new | 20 (3.6%) | 18 (3.3%) | 0.91 (0.49-1.70) |
| onset/recurrent) | | | |
| Symptomatic ICH | 24 (4.3%) | 19 (3.5%) | 0.80 (0.44-1.45) |
| Pneumonia | 17 (3.1%) | 25 (4.6%) | 1.49 (0.81-2.73) |
| Congestive | 4 (0.7%) | 9 (1.6%) | 2.28 (0.71-7.36) |
| cardiac failure | | | |
| Hypotension** | 1 (0.2%) | 7 (1.3%) | 7.09 (0.88-57.4) |
| Urinary tract infection | 7 (1.3%) | 8 (1.5%) | 1.15 (0.42-3.17) |
| Deep vein thrombosis/ | 8 (1.4%) | 3 (0.5%) | 0.38 (0.1-1.42) |
| pulmonary embolism | | | |
| Angioedema | 1 (0.2%) | 1 (0.2%) | 1.01 (0.06-16.1) |
| Hives/Urticaria/Pruritis | 0 | 0 | — |

*Unadjusted
Notes:
The safety population includes only patients who received any dose of study drug (N = 1101);
RR = risk ratio.
Symptomatic intracranial hemorrhage (ICH) includes the MedDRA PT codes: vascular procedure complication, hemorrhagic transformation of stroke, hemorrhagic stroke, hemorrhage intracranial, cerebral hemorrhage, subarachnoid hemorrhage
Pneumonia includes the MedDRA PT codes: Pneumonia, Aspiration pneumonia, Bacterial pneumonia.
Urinary tract infection includes the MedDRA PT codes: Urinary tract infection and Urosepsis
**1 case in the nerinetide group occurred 11 days post dose, the remaining hypotension events occurred on the same day as dosing.

### Example 2

This example investigates cleavage of nerinetide by plasmin and describes variant active agents inhibiting PSD-95 resistant to plasmin cleavage.

### Results

### Nerinetide is Cleaved by Plasmin

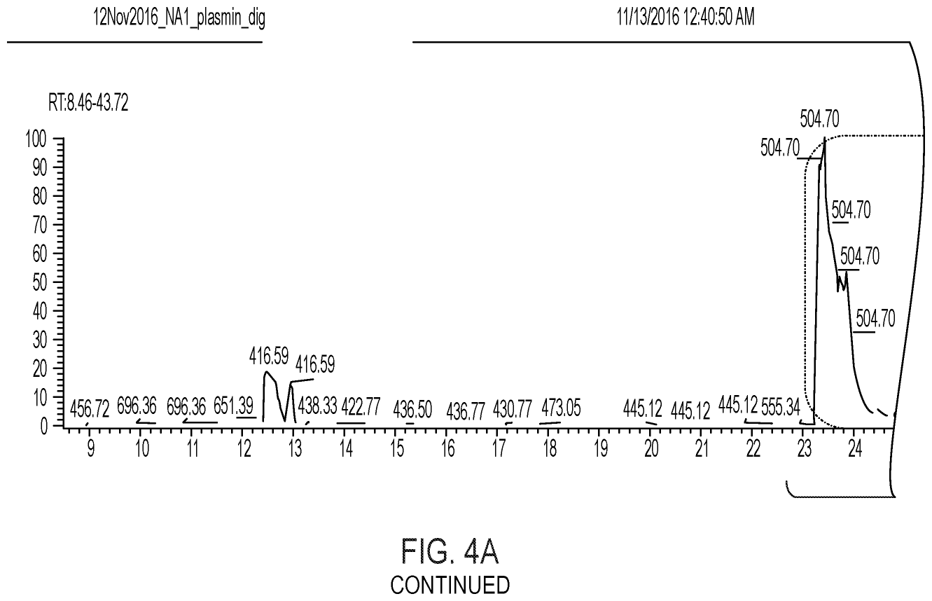

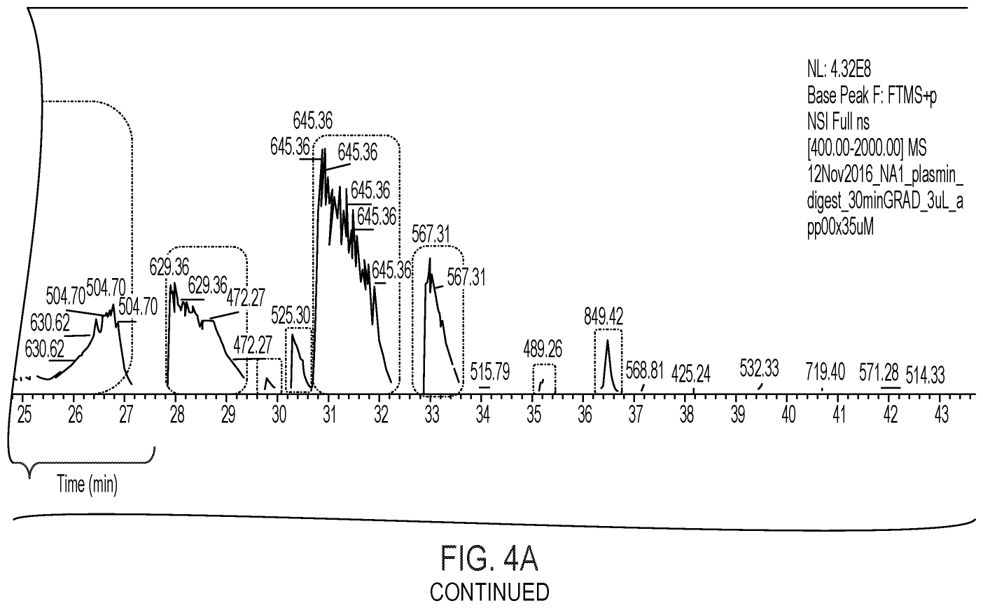

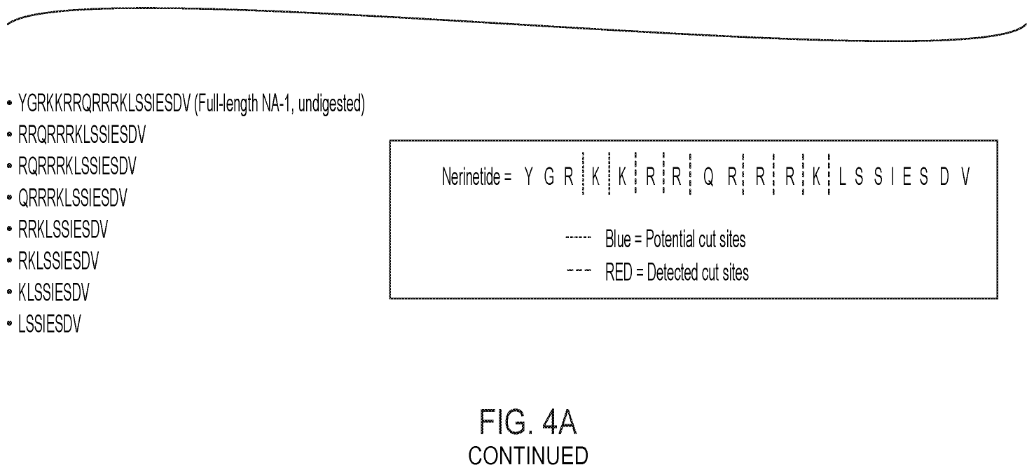

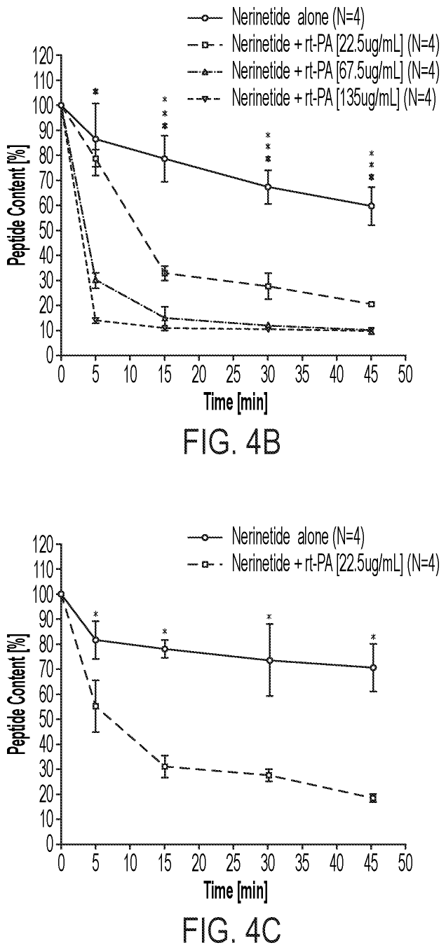

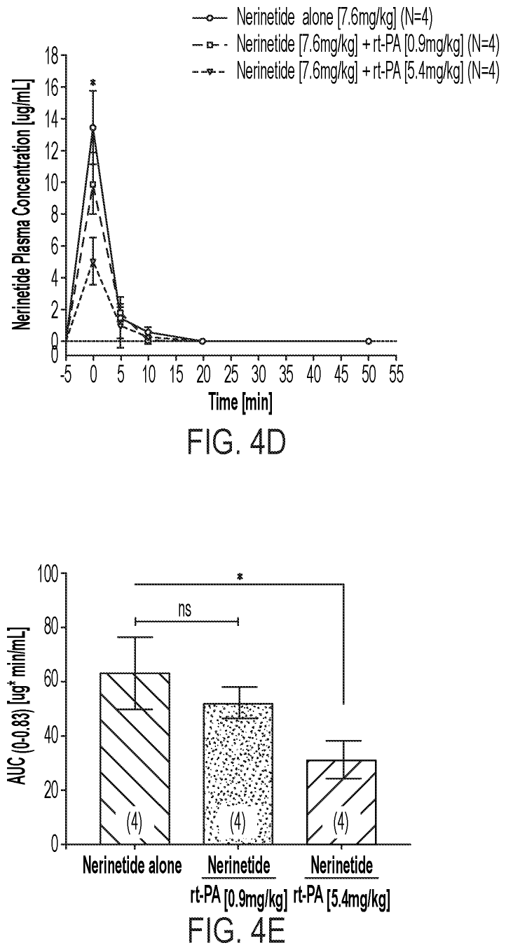

Nerinetide does not have any intrinsic fibrinolytic activity and does not affect the activity of thrombolytics such as alteplase or tenecteplase but the converse is different. Plasmin, a serine protease, is activated by thrombolytics to dissolve fibrin blood clots and persists for several hours( Chandler et al., Haemostasis 30, 204-218 (2000). Plasmin has a cleavage specificity on the C-terminal side of basic residues, and so may occur after residues 3, 4, 5, 6, 7, 9, 11 and 12 from the N-terminus of nerinetide. Cleavage products consistent with these sites of cleavage were observed after incubating nerinetide (18 mg/mL) with plasmin (1 mg/mL) in phosphate-buffered saline at 37° C. and analyzing the samples by LC/MS (FIG. 4A). We tested this directly in both rat and human plasma by incubating 65 ug/ml of nerinetide with alteplase in plasma at 37° C. and testing nerinetide levels by HPLC (FIG. 4B, C). The concentration of 65 ug/ml of nerinetide represents the theoretical peak concentration in a 75 kg person receiving 2.6 mg/kg dose as a bolus. Alteplase was added over 60 minutes to simulate the clinical dosing approach (Methods). Concentrations of alteplase (indicated in FIG. 4B [rat] and FIG. 4C [human]) were selected to simulate the peak concentrations anticipated in a person at the end of the initial 10% bolus of a 0.9 mg/kg dose (22.5 ug/ml), as well as 3 times and 6 times that dose in the rat, as the rat fibrinolytic system may be less sensitive to human recombinant tPA (Korninger, Thromb Haemost 46, 561-565 (1981)). The addition of alteplase reduced the nerinetide content in rat plasma in a concentration-dependent manner (FIG. 4B), and the effect of the “human equivalent” dose of 22.5 ug/ml alteplase was similar between the rat and human plasma (FIG. 4B, C).

Since the effect of nerinetide in the ESCAPE-NA1 trial was negated by alteplase, we next evaluated the effects of alteplase on pharmacokinetics (PK) of nerinetide in rats. Alteplase was administered at 0.9 mg/kg (human dose) and at 5.4 mg/kg (6 times the human dose) in an infusion that simulated the clinical protocol (10% bolus followed by a 60 min infusion of the remainder). Nerinetide was administered as an intravenous bolus at the start of the alteplase infusion at 7.6 mg/kg. This is the dose most commonly used in rats in prior stroke studies (5, 7, 15) and that leads to a Cₘₐₓ in rats similar to that produced in humans receiving 2.6 mg/kg, the dose used in ESCAPE-NA1. The co-administration of nerinetide with the human dose of alteplase resulted in a non-significant reduction of the Cmax and AUC of nerinetide (FIG. 4D, E). However, at six times the human dose (5.4 mg/kg) alteplase caused a significant lowering of the mean Cmax and AUC of nerinetide (49.5% and 44%, respectively). This finding in animals supports the PK data from the ESCAPE-NA1 trial in which alteplase-treated patients exhibited lower plasma levels of nerinetide.

The cleavage of full-length nerinetide by high dose alteplase was incomplete, raising the possibility that some active drug could still remain to achieve neuroprotection. This was supported in rats by a dose-response study of nerinetide in a model of transient middle cerebral artery occlusion (tMCAO). Nerinetide and lodoxamide was administered to rats intravenously as a bolus injection, 60 minutes after tMCAo. FIG. 11A shows hemispheric infarct volume measurements 24 hours after tMCAo. Bars in A and represent mean±SD, with all individual data points plotted. Asterisks in A indicate P<0.01 when compared to the vehicle group (one-way ANOVA post hoc Tukey's correction for multiple comparisons test) N=12-14 animals/group. FIG. 11B shows neurological scores 24 hours after tMCAo. Significant differences are indicated with an asterisk when compared to the vehicle group (Kruskal-Wallis analysis of variance on ranks with a post-hoc Dunn's correction for multiple comparisons test, *P<0.01). Vehicle: PBS alone. Scrambled: ADA peptide incapable of binding PSD-95. Doses as low as 0.25 mg/kg produced a significant reduction in infarct volume (P=0.01) and an improvement in neurological function. Doses of as little as 0.025 mg/kg were also effective. Doses up to at least 25 mg/kg were also effective with the highest efficacy being at about 15 mg/kg. The observed wide therapeutic range was attributable to nerinetide, and not to the mast cell degranulation inhibitor lodoxamide, which was present in all solutions to avoid potential hypotension due to histamine release.

### Dose Separation Restores the Treatment Benefit of Nerinetide

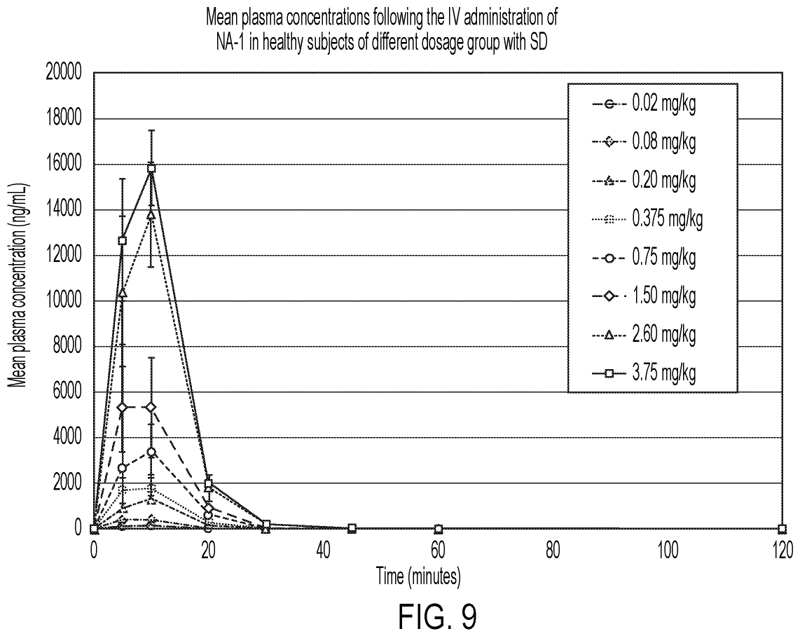

In both rat and human at the human equivalent concentrations, the half-life of nerinetide was approximately 5-10 minutes (FIG. 4D), which is similar to the half-life of nerinetide in healthy human volunteers (FIG. 9). The short half-life of nerinetide in rats and humans is not explained by degradation, because degradation in plasma is slow (compare FIGS. 4B and 4D). This suggests that nerinetide exits the intravascular compartment rapidly as it partitions into other tissues. If so, then administering nerinetide before alteplase is given could eliminate its cleavage in the blood stream and preserve its neuroprotective benefit.

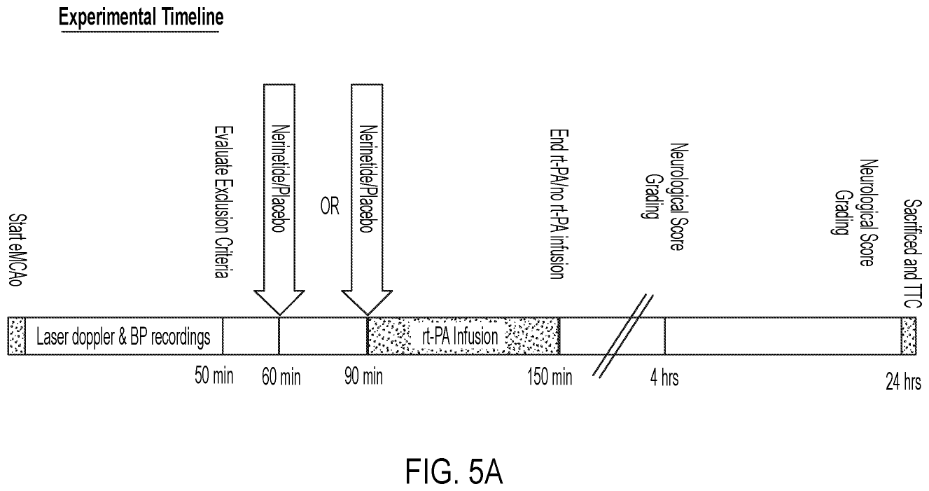

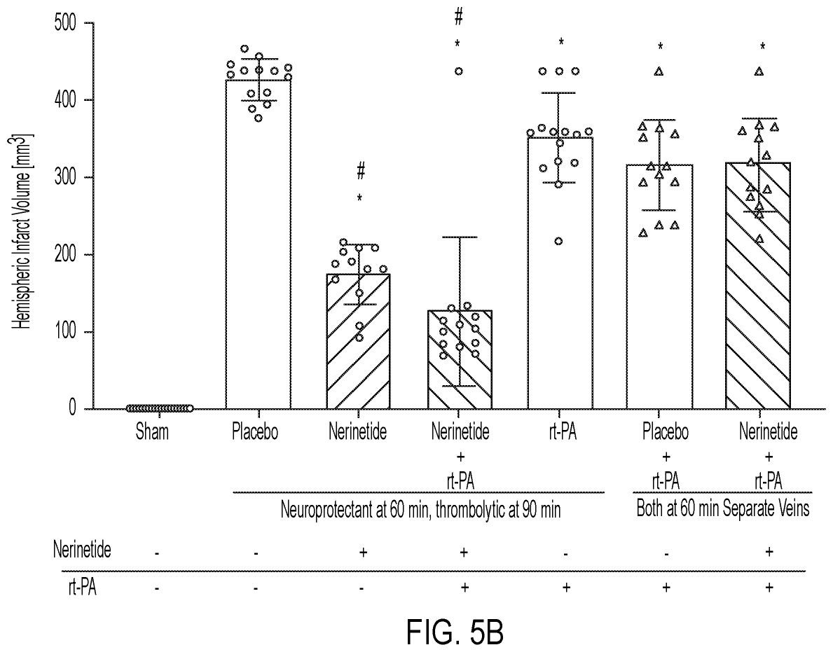

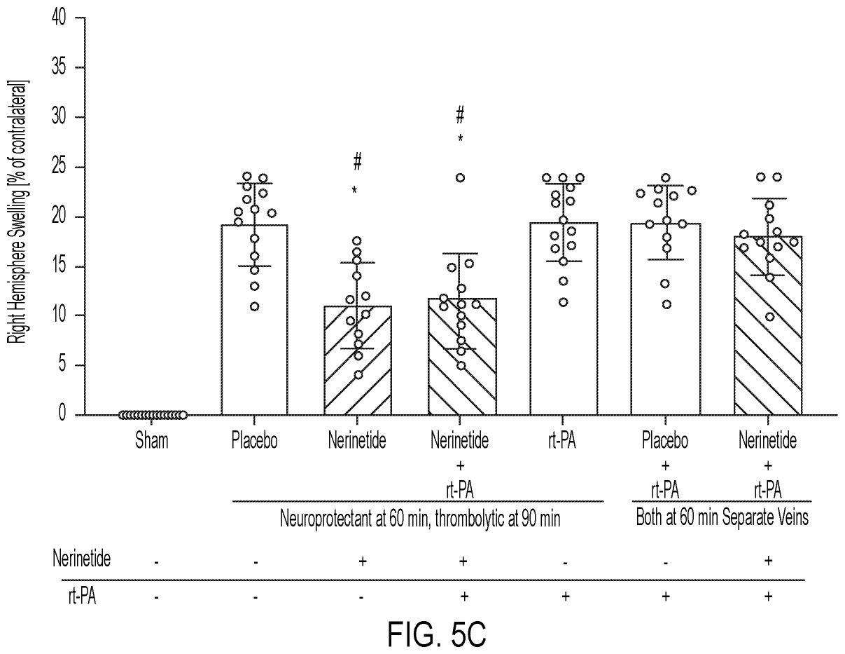

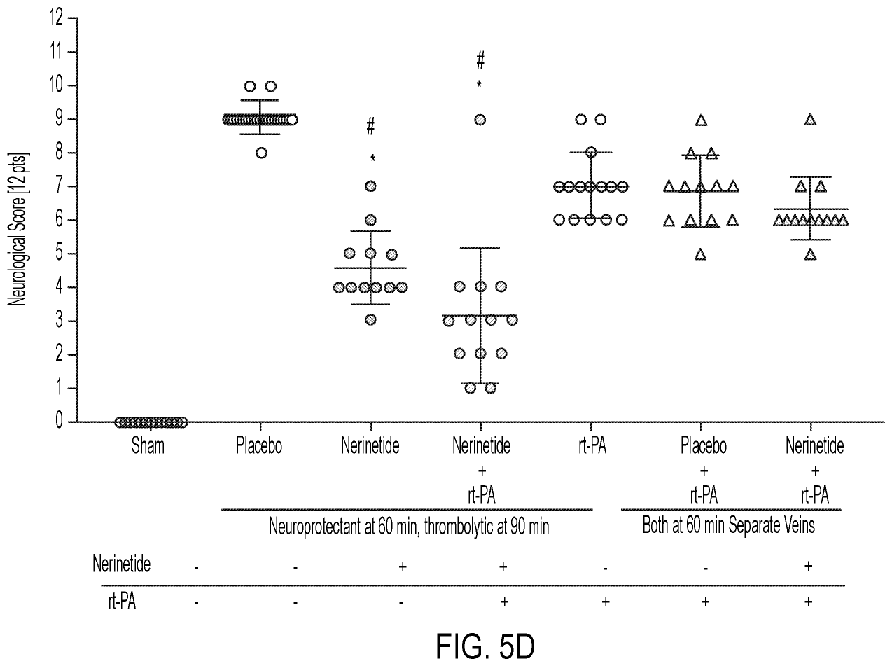

To test this, male Sprague-Dawley rats (10-12 weeks old; 270-310 g; Charles River, Montreal, QC, Canada) were subjected to embolic middle cerebral artery occlusion (eMCAO), produced by the introduction of an autologous blood thrombus into the middle cerebral artery. Reperfusion was achieved by treatment with intravenous alteplase at a total dose of 5.4 mg/kg beginning at 90 minutes after ischemia onset. Alteplase was administered using the human injection protocol in which 10% of the total dose is given as a bolus, with the remainder 90% of the dose being given over a 60-minute infusion. The dose of alteplase was 6 times the human dose, in anticipation that the rat fibrinolytic system may be less sensitive to human recombinant tPA. This dose was chosen because in pilot studies, higher doses of alteplase (10× human dose) produced unacceptable mortality rates due to hemorrhagic conversions of strokes. Nerinetide was administered either 30 minutes prior to, or concurrently with, the start of the alteplase administration (FIG. 5A) at a dose of 7.6 mg/kg. This dose results in PK parameters (Cₘₐₓ and AUC) similar to those achieved in humans receiving the clinically effective dose of 2.6 mg/kg (Compare FIG. 4D with FIG. 9). Infarct volumes, hemispheric swelling and neurological scores were evaluated at 24 hours.

Nerinetide alone, administered 60 minutes after eMCAO, reduced infarction volume by 59.2% (from 427±27 mm³ to 175±40 mm³) whereas alteplase alone reduced infarction volume by 26% when given at 60 min and 18% when given at 90 minutes after eMCAO (FIG. 5B). The beneficial effect of nerinetide was eliminated completely when it was administered concurrently with alteplase at 60 min after eMCAO. By contrast, nerinetide was highly effective when its administration at 60 minutes was followed by alteplase 30 minutes later (70% infarct volume reduction). This beneficial effect of a 30-minute dose separation between nerinetide and alteplase was similarly reflected in reducing hemispheric swelling (FIG. 5C) and in improving neurological scores (FIG. 5D) after eMCAO. There were no differences in physiological parameters, mortalities, or exclusions between the groups.

We conducted further PK studies to probe the necessary dose-separation interval to mitigate degradation. These studies were conducted in cynomolgus macaques (Macaca fascicularis) to maximize their relevance to humans. Nerinetide was given as a 10-minute intravenous infusion at a dose of 2.6 mg/kg. This dosing regimen was neuroprotective in macaques exposed to stroke by LVO (Cook et al., Nature 483, 213-217 (2012)) and was used in both the Phase 2 ENACT trial (Lancet Neurol 11, 942-950 (2012)) and the ESCAPE-NA1 trial (Lancet 395, 878-887 (2020)). We examined the scenarios in which alteplase administration was started simultaneously with the nerinetide infusion start, at the end of the 10-minute nerinetide infusion, or 10 minutes after the end of nerinetide infusion. Alteplase (1 mg/kg) was administered through a separate intravenous line as a 10% bolus, followed by an infusion of the remaining 90% over 1 hour, as per its clinical use.

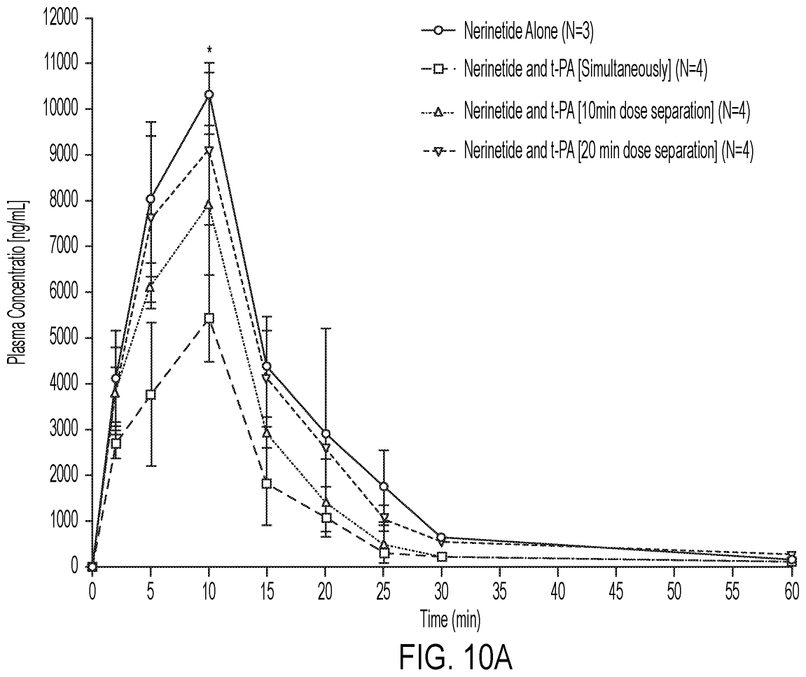

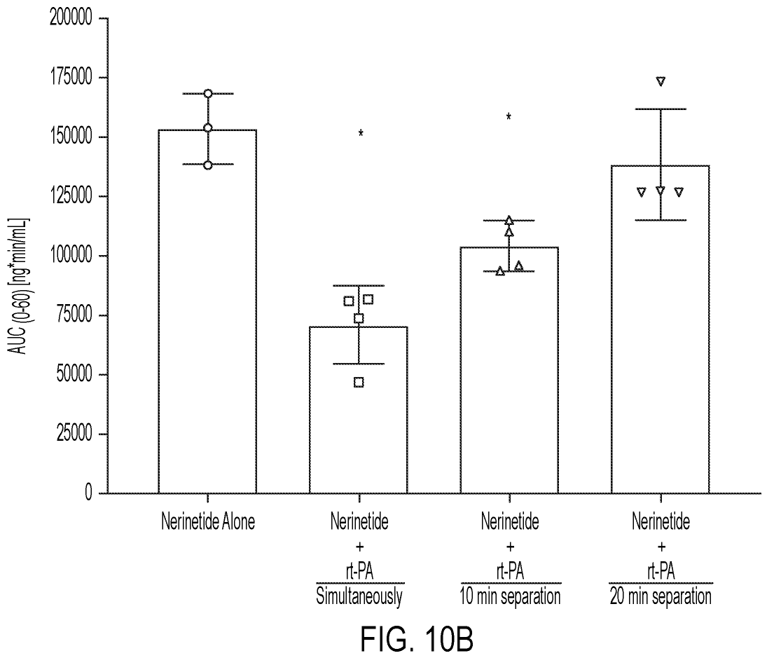

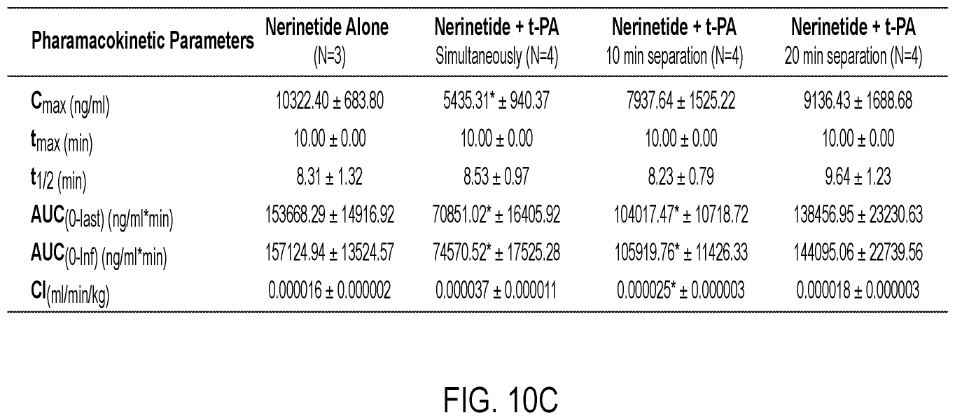

The co-administration of nerinetide with alteplase resulted in a 47.4% reduction of the Cₘₐₓ and 53.9% reduction in the AUC of nerinetide (FIGS. 10A-C). Starting alteplase at the end of the nerinetide infusion resulted in a modest 23.1% reduction of the Cmax and 32.3% reduction in the AUC but still achieved a plasma concentration likely to be effective based on animal models. Waiting 10 minutes following the end of the 10-min nerinetide infusion (or equivalently waiting 20 min from the start of the infusion) eliminated degradation of Cₘₐₓ or AUC by alteplase to within the margin of measurement error indicated by the error bars (FIGS. 10A-C).

Based on these results, a dose-separation approach is a practical strategy to preserve neuroprotection by nerinetide in animals treated with alteplase.

### D-amino Acids Render Nerinetide Insensitive to Cleavage by Thrombolytics

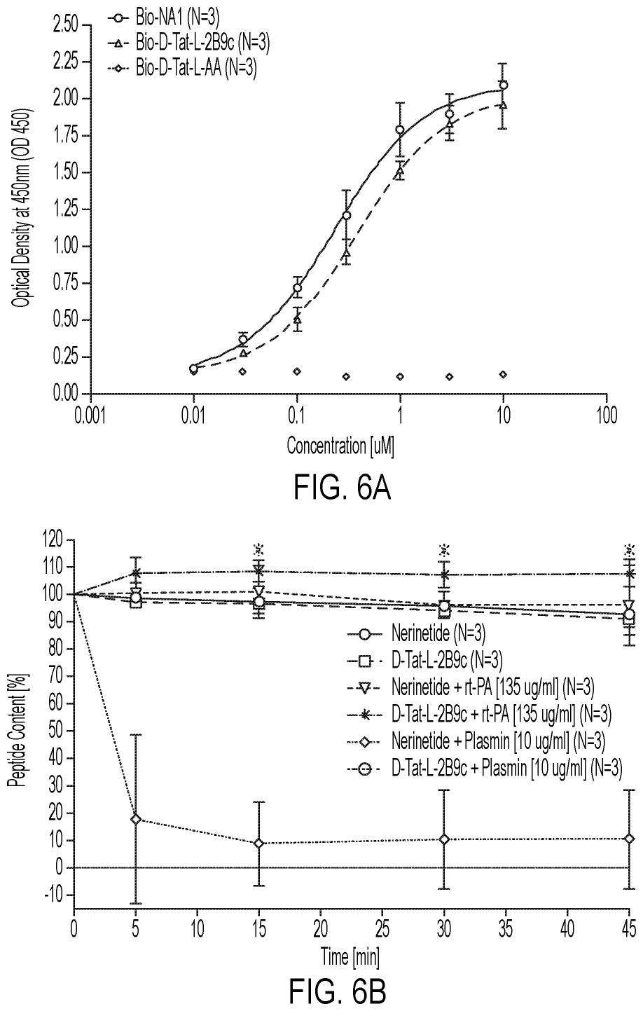

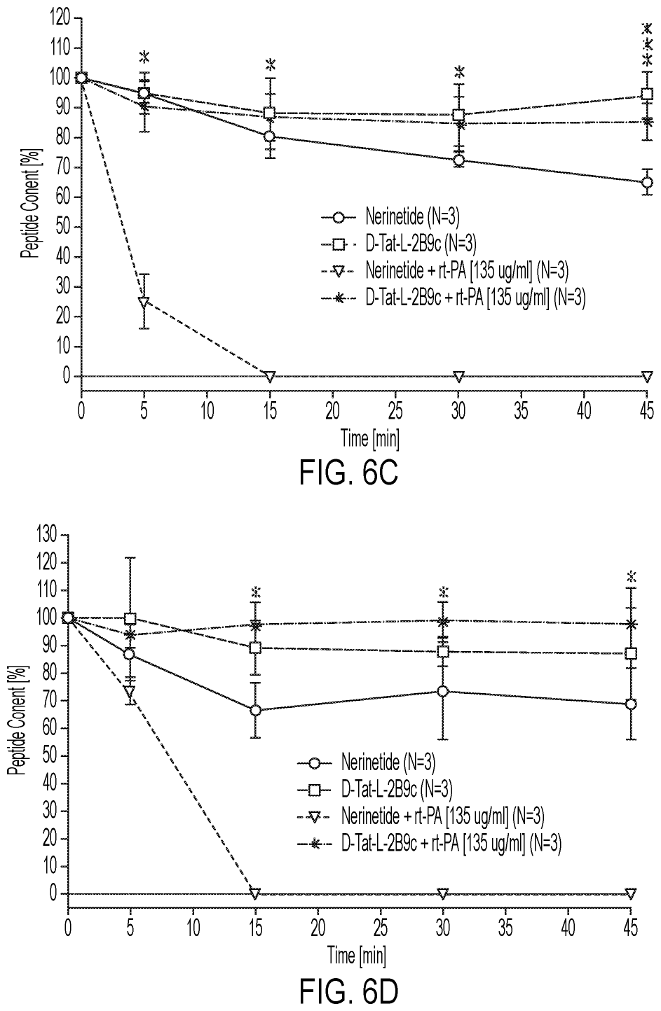

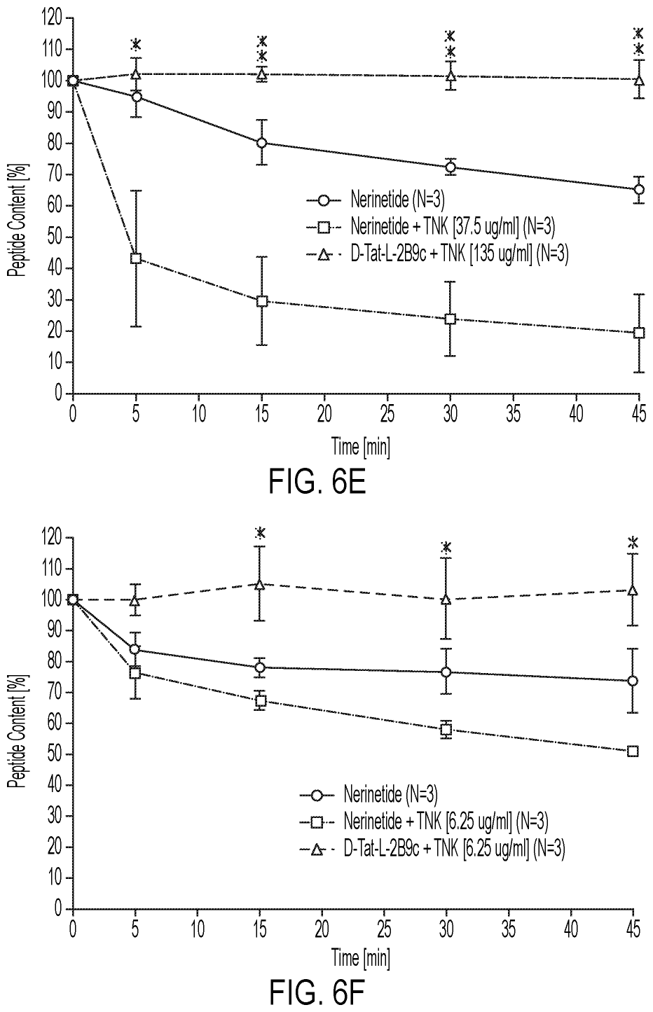

We reasoned that while specific binding to PSD-95 PDZ2 may require the L-enantiomeric configuration of the C-terminal amino acids, the Tat portion could be rendered resistant to protease degradation by substituting L- for D-amino acids. In so doing, we generated a peptide termed D-Tat-L-2B9c comprising 11 D-amino acids of Tat fused to the 9 L-amino acids of the GluN2B C-terminus (ygrkkrrqrrrKLSSIESDV SEQ ID NO:89). This peptide had substantially similar binding as nerinetide to the target PDZ2 domain of PSD95 in ELISA assays (FIG. 6A). The binding was specific, as the same D-Tat-L-2B9c construct containing a double point mutation in the last 3 C-terminal residues (Lys-Leu-Ser-Ser-Ile-Glu-Ala-Asp-Ala (SEQ ID NO:90); termed D-Tat-L-2B9c_{AA}) failed to bind.

Nerinetide or D-Tat-L-2B9c alone are stable in phosphate buffered saline at 37° C., but incubating nerinetide with plasmin resulted in its rapid degradation (FIG. 6B). By contrast, D-Tat-L-2B9c showed no significant degradation under the same conditions. Neither were affected by co-incubation with alteplase (FIG. 6B) because plasminogen, not nerinetide, is the direct substrate for alteplase. Similarly, both nerinetide and D-Tat-L-2B9c alone were stable in both rat and human plasma in the absence of alteplase (FIG. 6C, D). However, the addition of alteplase (rt-PA; 135 ug/ml) resulted in the rapid degradation of nerinetide, but not of D-Tat-L-2B9c (FIG. 6C, D). We also conducted similar experiments with tenecteplase (TNK), a tissue plasminogen activator currently in use for the treatment of acute myocardial infarction that may gain popularity for stroke. The addition of TNK to both rat and human plasma resulted in the rapid elimination of nerinetide, but not D-Tat-L-2B9c (FIG. 6E, F).

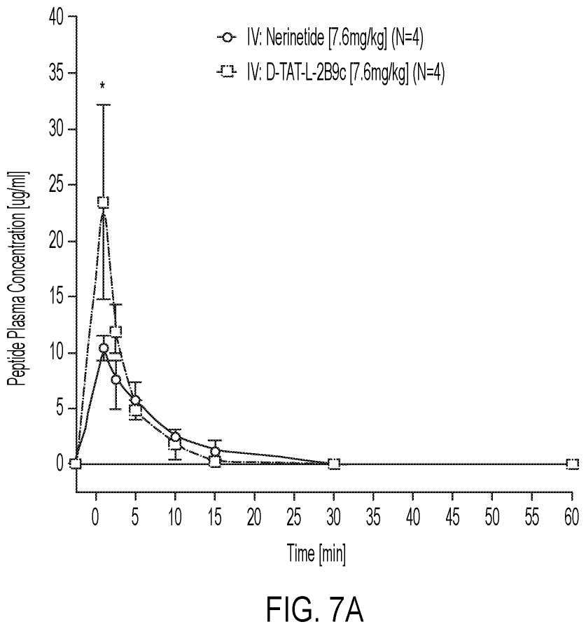

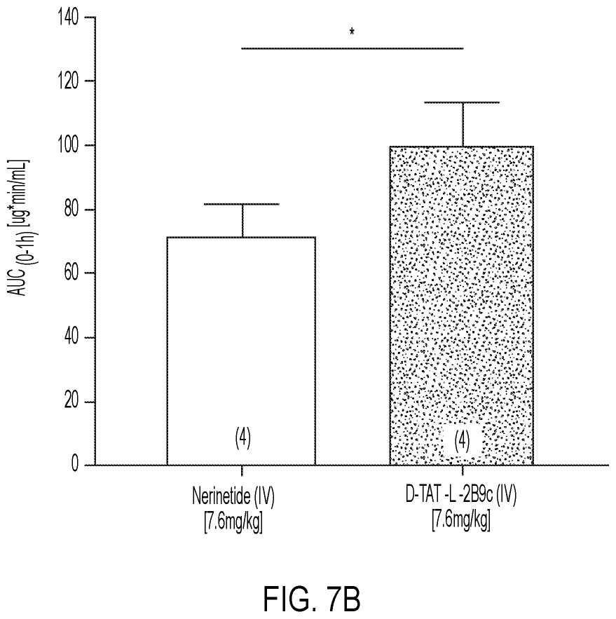

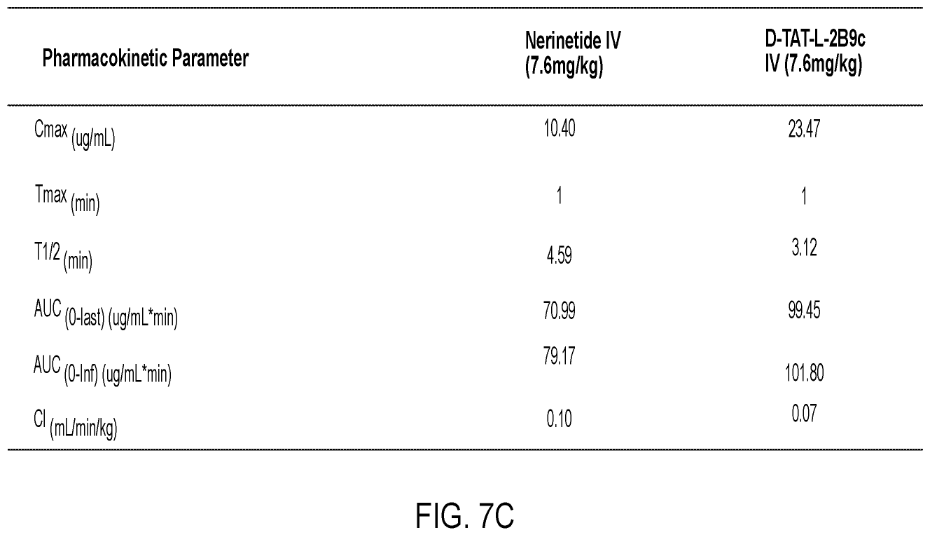

When administered as an intravenous bolus to rats, nerinetide and D-Tat-L-2B9c both exhibited substantially similar pharmacokinetic profiles, slightly favoring D-Tat-L-2B9c (higher cₘₐₓ and AUC). In the absence of thrombolytic agents, the rapid disappearance of both from the intravascular compartment (FIGS. 7A-C) despite their relative plasma stability (FIG. 6C-F) supports the hypothesis that the pharmacokinetics of both are governed more by a rapid distribution into tissues than by proteolytic breakdown.

D-Tat-L-2B9c is an Effective Neuroprotectant when Co-Administered with Alteplase

D-Tat-L-2B9c and nerinetide were equally effective in reducing infarction volume, reducing hemispheric swelling, and improving neurological scores in the rat model of tMCAO. We therefore examined whether the effectiveness of D-Tat-L-2B9c would be preserved with a concurrent administration of alteplase.

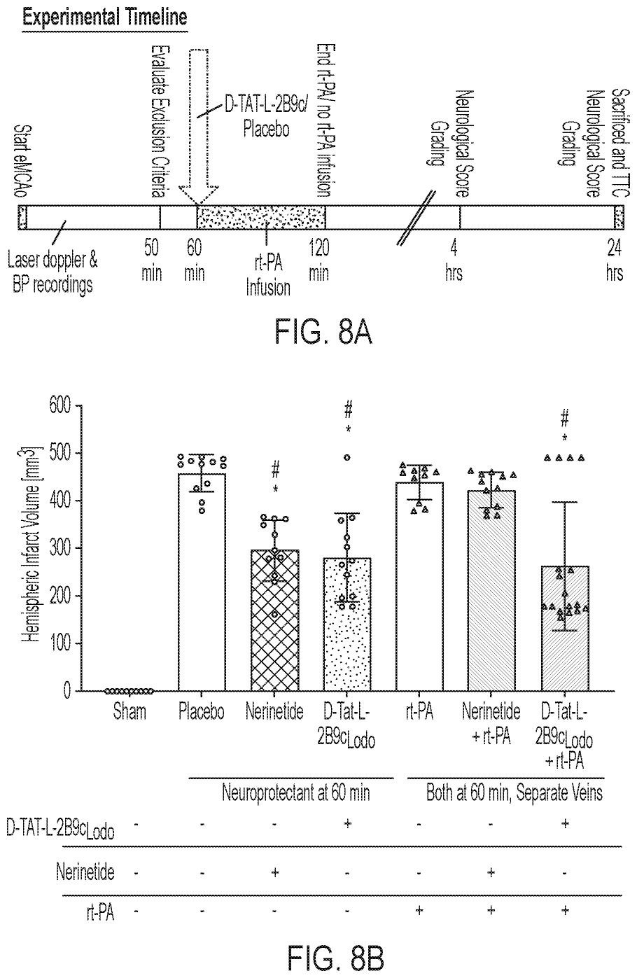

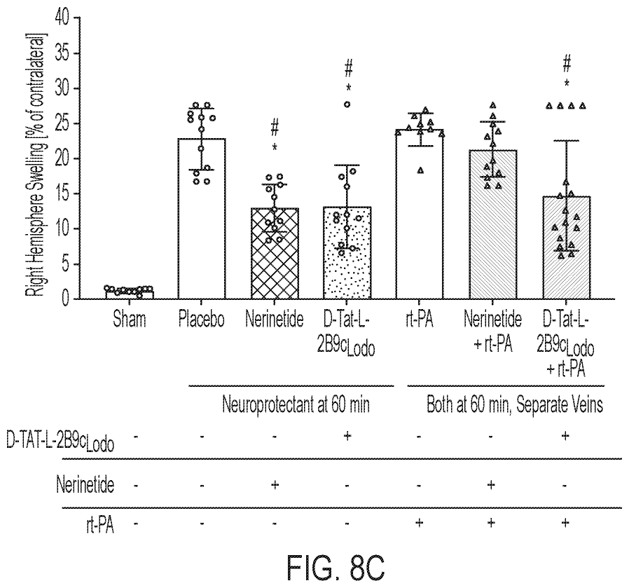

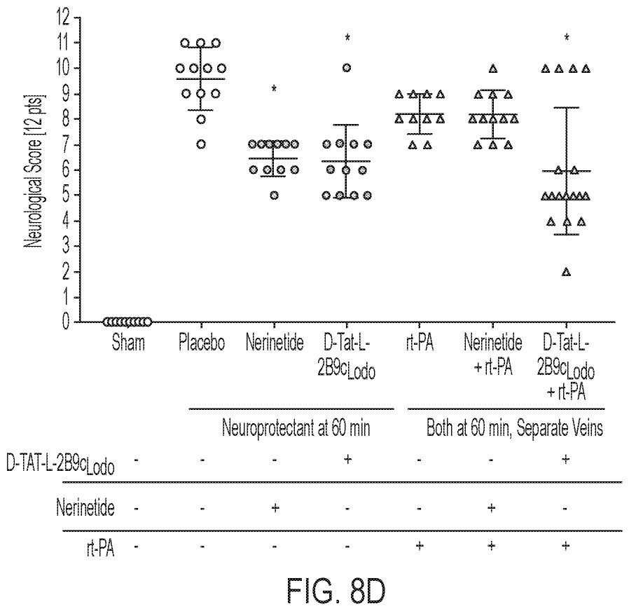

Male Sprague-Dawley rats were subjected to eMCAO as already described. Nerinetide (7.6 mg/kg) or D-Tat-L-2B9c (7.6 mg/kg) were given as bolus injections at 60 minutes. Alteplase (5.4 mg/kg over 60 min) was also started at 60 min after eMCAO, concurrently with the active agent that inhibits PSD-95. Neurological scoring, infarct volume, and hemispheric swelling were assessed at 24 hours (FIG. 8A).

Nerinetide alone, administered 60 minutes after eMCAO, reduced infarction volume substantially in the absence of alteplase (from 458±39 mm³ to 296±66 mm³). This effect was eliminated completely when both nerinetide and alteplase were given (FIG. 8B). By contrast, treatment with D-Tat-L-2B9c was as effective as nerinetide alone in the absence of alteplase, and this effect persisted when both D-Tat-L-2B9c and alteplase were given together (FIG. 8B). The beneficial effect of D-Tat-L-2B9c was evident when measuring infarct volumes (FIG. 8B), hemispheric swelling (FIG. 8C) and neurological scores (FIG. 8D). There were no differences in physiological parameters, mortalities, or exclusions between groups.

### Discussion

We have shown that administering nerinetide a short period of time prior to the initiation of alteplase treatment completely eliminates the inactivation of nerinetide by alteplase (FIGS. 6A-F). This approach is driven by PK considerations which are similar between humans and rats (FIG. 4D) and is agnostic to inter-species differences in fibrinolytic biology. Due to its short half-life in plasma, nerinetide exits the intravascular compartment and is no longer subject to substantial cleavage by alteplase when the latter is administered 30 minutes thereafter.

As an alternative to dose separation, the protein-protein interactions of PSD-95 could be addressed with a protease-insensitive inhibitor. We have shown that a practical approach to rendering nerinetide insensitive to cleavage by thrombolytics is to convert the plasmin-sensitive residues (i.e., at least the Tat protein transduction domain) into D-amino acids. The consensus sequence terminating with the PDZ-domain binding [T/S]-XV motif was preserved, resulting in both nerinetide and D-Tat-L-2B9c having equivalent binding to PSD-95 and neuroprotective efficacy.

An agent such as D-Tat-L-2B9c might be administrable as soon as a stroke is identified, even before arrival to hospital as is currently the case for nerinetide in the FRONTIER trial. It might also be administered at any other time in the care path of a stroke patient, before, concurrently with, or after the administration of a thrombolytic agent if this is deemed appropriate by the treating medical professional.

### Materials and Methods

### Animals

Experiments were conducted on anaesthetized, male Sprague-Dawley rats, 10-12 weeks old and weighing between 270-320 g (Charles River; Montreal, QC, Canada). The rats were housed in sterile cages and allowed free movement and access to food and water ad lib throughout the experiment.

### Study Drugs

Nerinetide was synthesized and formulated at 18 mg/ml by NoNO Inc. (Toronto, Canada. The placebo was comprised of phosphate-buffered saline supplied in visually identical vials. Lyophilized D-TAT-L-2B9c was synthesized by Genscript (China) and subjected to peptide hydrolysis and amino acid liquid chromatography analysis to obtain a precise measure of peptide content. Reconstituted peptides were stored at −20° C. until used. Human rt-PA (Alteplase/CathFlo; Roche, San Franscisco, U.S.A) was reconstituted to a final concentration of 1 mg/ml in sterile water for injection (USP 3 ml, AirLife, AL7023) and stored at 2 to 8° C. until used. TNK (50 mg powder for solution, Hoffmann-La Roche Limited) for the stability studies was reconstituted to a final concentration of 37.5 ug/ml or 6.25 ug/m in sterile water for injection (SWFI) and stored at 2 to 8° C. ° C. until used. In all animal experiments, nerinetide or D-Tat-L-2B9c was given as a bolus injection. The mast cell degranulation inhibitor lodoxamide was co-administered (0.1 mg/kg) with both to avoid potential hypotension due to histamine release, a potential effects of cationic peptides. rt-PA in all experiments was administered over 60 minutes (10% as a bolus followed by a 60 min infusion of the remaining 90%).

### Other Reagents

All were purchased from Sigma-Aldrich (Oakville, ON, Canada), unless specified otherwise. HPLC grade acetonitrile, trifluoroacetic acid and water were purchased from Fisher Scientific (Fair Lawn, N.J., USA). TRIS, perchloric acid, and phosphate buffered saline were obtained from Sigma-Aldrich (St. Louis, Mo., USA). Commercial rat plasma (Innovative Research Inc, Rat Sprague Dawley plasma with NA-EDTA [Catalog No: IRTSDPLANAE10 ML]) and human plasma (Innovative Research Inc, Pooled Human plasma with NA-EDTA [Catalog No: IPLANAE10 ML]) were used.

### Stroke Studies

The studies were designed to have 80% power to detect a 40% absolute difference between control and treatment groups at p=0.05. Animal randomization, drug allocation and treatment drug preparation were performed by a research associate not directly involved with surgical or outcome assessment. Nerinetide and D-Tat-L-2B9c were freshly prepared at a concentration of 7.6 mg/mL in 500 uL aliquots. Alteplase was prepared from lyophilized drug and, like matching placebo, stored in identical glass tubes. Drugs were kept at 4° C. until 10 minutes prior to use. The surgeon and the investigators responsible for the surgery, stroke volume measurement, behavioural assessment and statistical analysis were blinded to treatment allocation.

All animals subjected to surgery had their physiological parameters measured prior to MCA occlusion. PE-50 polyethylene tubing was inserted into the right femoral artery for invasive monitoring of mean arterial blood pressure and for obtaining blood samples to measure blood gases (pH, PaO2, and PaCO2), electrolytes (Na⁺, K⁺, iCa) and plasma glucose at baseline [Blood gas cartridge CG8+, VetScan i-STAT 1 Analyzer]. Body temperature was monitored continuously with a rectal probe and maintained at 37.0±0.7° C. with a heating lamp. tMCAO was performed as previously described (5, 7). eMCAO was achieved as described by Henninger et al., Stroke 37, 1283-1287 (2006). In brief, a 18-22 mm long autologous blood clot produced from whole blood withdrawn 24 hours before occlusion from the same rat was introduced into the middle cerebral artery by extrusion from PE tubing introduced into the internal carotid artery. Relative regional cerebral blood flow (rCBF) measurements with a laser Doppler monitor (Perimed, Järfälla, Stockholm, Sweden) were used to confirm successful eMCAO (>65% drop in rCBF) as well as reperfusion with alteplase.

Infarct volumes and hemispheric swelling were evaluated at 24 hours post-stroke from standard brain slices stained with 2% 2,3,5-triphenyltetrazolium chloride (Sigma Aldrich, St. Louis, Md., USA)(7). Neurologic scoring was conducted at 24 hours after stroke onset using forelimb-placing tests comprising of frontal visual placing, sideways visual placing, frontal tactile placing, sideways tactile placing, and vertical tactile placing (scores range from 0-2 in each component for a maximum of 12 indicating maximum impairment.

### In-Vitro Peptide Degradation Assay

To determine nerinetide stability in the presence of rt-PA in plasma, we use an in-vitro peptide content analysis by HPLC. In brief, Nerinetide or D-Tat-L-2B9c were spiked into rat or human plasma at a concentration of 65 ug/ml. rt-PA was added after the baseline time-point was collected, at the specified concentrations. rt-PA administration followed the clinical dosing approach [10% bolus dose followed by 60-mins infusion (90% of the dose)], using a Harvard apparatus pump. Sample collection following IV bolus was performed at 5 min, 15 min, 30 min and 45 min post-dose. At each time point, approximately 100 uL of plasma was collected from each vial using a fresh syringe. Plasma was then collected and stored at -80° C. until analyzed.

### In-Vivo Pharmacokinetic Analysis

The goal of these study was to evaluate nerinetide PK parameter changes when in the presence of circulating rt-PA and plasmin. Male naïve rats received an intravenous administration of either nerinetide alone, nerinetide plus rt-PA (0.9 mg/kg) or nerinetide plus rt-PA (5.4 mg/kg). Sample collection was performed pre-dose and 0 min, 5 min, 10 min, 20 min, 50 min post-dose. At each time point, approximately 300 uL of blood was sampled from each animal using a fresh syringe. Blood samples were collected in previously prepared Eppendorf tubes [30 ul of EDTA 2.5%] and centrifuged for 20 minutes to separate plasma and cell components. Plasma samples was then collected and stored at −80° C. until analyzed by HPLC.

### High Pressure Liquid Chromatography

Plasma samples were stored at −80° C. until analyzed. Nerinetide or D-Tat-L2B9c was extracted by precipitation with 1M perchloric acid. All analyses were performed on an Agilent 1260 Infinity Quaternary LC System (Agilent Technologies, Santa Clara, Calif., USA) and on a 25 cm [YMAA12S052546WT] C-18 RP-HPLC column (Agilent Technologies, Santa Clara, Calif., USA). The column was equilibrated with 10% acetonitrile with 0.1% TFA at 40° C. The eluent flow was 1.5 ml/min (gradient from 10% to 35% acetonitrile in 0.1% TFA) The UV trace was recorded at 220 nm. Concentrations of nerinetide or D-Tat-L-2B9c were derived from calibration standards obtained by spiking the agent into plasma.

### ELISA Assays

ELISA plates were coated with 1 ug/ml PSD95PDZ2 in 50 mM bicarbonate buffer overnight at 4 C. The plate was blocked in 2%BSA in PBST (0.05%) for 2 h at room temperature. It was then incubated with biotinylated ligand (nerinetide, D-tat-L2B9c or D-Tat-L-AA) at the indicated concentrations (FIG. 4A) and incubated overnight at 4 C. After washing with PBS-T, the plate was incubated for 30 min with (1:3000) SA-HRP, washed again, and incubated with TMB solution for 10 min. The reaction was stopped with 100 ul H2504. Absorbance was determined at 450 nm with the synergy H1 reader.

### Statistics

Changes in peptide concentration were analyzed using a two-way repeated measures ANOVA, followed by the Sidak correction for multiple comparisons. The pharmacokinetic (PK) parameters of peak plasma concentration (Cmax) and the area under the plasma concentration-time curve from 0 to last measured concentration (AUC) were obtained with PKsolver Software (USA) using a non-compartmental analysis and employing a linear interpolation. For stroke studies, differences between groups were tested using a One-way ANOVA with a Tukey's correction for multiple comparisons. Differences between groups on the neurological score assessment were analyzed using the non-parametric Kruskal-Wallis analysis of variance on ranks with a post-hoc Dunn's correction. Values for animals experiencing premature death due to any reason including subarachnoid hemorrhage or hemorrhagic transformation were imputed to reflect the worst neurological score and the maximum stroke volume achieved across all animals.

What is claimed is:

1. A method of treating a population of subjects having or at risk of ischemia comprising administering to the subjects an active agent that inhibits PSD-95, cleavable by plasmin, and reperfusion, wherein the population of subjects includes:
subjects administered the active agent that inhibits PSD-95 and mechanical reperfusion or a vasodilator agent or a hypertensive agent to effect reperfusion; and/or
subjects administered the active agent that inhibits PSD-95 and a thrombolytic agent to effect reperfusion, wherein the active agent that inhibits PSD-95 is administered at least 10 minutes before the thrombolytic agent,
and the population of subjects lacks:
subjects in which a thrombolytic agent is administered less than 3 hours before or less than 10 minutes after administering the active agent that inhibits PSD-95.

2. The method of claim 1, wherein the subjects have ischemic stroke.

3. The method of claim 1 or 2, in which the population lacks subjects in which the thrombolytic agent is administered less than four hours before the active agent that inhibits PSD-95 or less than 10 minutes after the active agent that inhibits PSD-95.

4. The method of claim 1 or 2, in which the population lacks subjects in which the thrombolytic agent is administered less than eight hours before the active agent that inhibits PSD-95 and less than 10 minutes after administering the active agent that inhibits PSD-95.

5. The method of claim 1 or 2, in which the population lacks subjects in which the thrombolytic agent is administered before the active agent that inhibits PSD-95 or less than ten minutes after administering the active agent that inhibits PSD-95.

6. The method of claim 1 or 2, in which the population lacks subjects in which the thrombolytic agent is administered before the active agent that inhibits PSD-95 or less than 20 minutes after administering the active agent that inhibits PSD-95.

7. The method of claim 1 or 2, in which the population lacks subjects in which the thrombolytic agent is administered before the active agent that inhibits PSD-95 or less than 30 minutes after administering the active agent that inhibits PSD-95.

8. The method of claim 1 or 2, in which the population lacks subjects in which the thrombolytic agent is administered before the active agent that inhibits PSD-95 or less than 60 minutes after administering the active agent that inhibits PSD-95.

9. The method of any preceding claim, wherein the population of subjects includes subjects administered the active agent that inhibits PSD-95 and mechanical reperfusion without receiving a thrombolytic agent.

10. The method of claim 1 or 2, wherein the population of treated subjects consists of:
(a) subjects administered the active agent that inhibits PSD-95 and mechanical reperfusion, a vasodilator agent or a hypertensive agent without a thrombolytic agent; and
(b) subjects administered the active agent that inhibits PSD-95 and a thrombolytic agent, wherein the thrombolytic agent is administered at least 10, 20, 30, 40, 50, 60, or 120 minutes after the active agent that inhibits PSD-95.

11. The method of claim 10, wherein at least some of the subjects according to item (b) also are administered mechanical reperfusion.

12. The method of claim 1 or 2, wherein the population includes subjects in which the thrombolytic agent is administered more than 3 or 4.5 hours after onset of stroke when the subjects were determined to be eligible for treatment with the thrombolytic agent less than 3 hours after onset of stroke.

13. The method of any preceding claim, wherein the population includes subjects administered the active agent that inhibits PSD-95 intranasally or intrathecally.

14. The method of any preceding claim, wherein the population includes at least 100 subjects.

15. The method of any preceding claim, wherein the population includes subjects in which the active agent that inhibits PSD-95 is administered over a ten minute period and the thrombolytic agent is administered at least 20 minutes from the start of administering the active agent.

16. The method of any preceding claim, wherein the active agent is a peptide of all L-amino acids.

17. The method of any preceding claim, wherein the active agent is nerinetide.

18. A method of treating a population of subjects receiving endovascular thrombectomy for ischemic stroke comprising:
administering both an active agent that inhibits PSD-95, cleavable by plasmin, and a thrombolytic agent to some of the subjects, wherein the active agent that inhibits PSD-95 is administered at least 10, 20, 30, 40, 50, 60 or 120 minutes before the thrombolytic agent, and
administering the active agent that inhibits PSD-95 or the thrombolytic agent but not both to the other subjects of the population.

19. The method of claim 18, wherein the subjects receiving the active agent that inhibits PSD-95 and the thrombolytic agent do so before the subjects receive endovascular thrombectomy.

20. The method of claim 18 or 19, wherein the subjects receiving the active agent that inhibits PSD-95 or the thrombolytic agent but not both do before the subjects receive endovascular thrombectomy.

21. The method of any one of claims 18-20, wherein in the subjects receiving both the active agent that inhibits PSD-95 and thrombolytic agent, the active agent that inhibits PSD-95 is administered at least 10 minutes before the thrombolytic agent, and the active agent that inhibits PSD-95 or the thrombolytic agent but not both is administered to the other subjects.

22. A method of treating a population of subjects having or at risk of ischemia, comprising administering to the subjects an active agent that inhibits PSD-95, and a thrombolytic agent, wherein the population of subjects includes:
subjects administered a first active agent that inhibits PSD-95 cleavable by plasmin and a thrombolytic agent, wherein the first active agent that inhibits PSD-95 is administered at an interval selected from at least 10, 20, 30, 40, 50, 60 or 120 minutes before the thrombolytic agent; and
subjects administered a second active agent that inhibits PSD-95 resistant to cleavage by plasmin and a thrombolytic agent, wherein the thrombolytic agent is administered before or within the interval after the active agent that inhibits PSD-95.

23. A method of treating a subject suspected of having ischemic stroke, comprising:
determining eligibility of the subject for treatment with a thrombolytic agent;
administering an active agent that inhibits PSD-95, cleavable by plasmin; and
at least 10, 20, 30, 40, 50, 60 or 120 minutes thereafter administering the thrombolytic agent.

24. The method of claim 23, wherein the active agent that inhibits PSD-95 is administered over a ten minute period and the thrombolytic agent is administered at least 20 minutes from the start of administering the active agent.

25. The method of claim 23 or 24, wherein the active agent is a peptide of all L-amino acids.

26. The method of claim 25, wherein the active agent is nerinetide.

27. The method of any one of claims 23-26, wherein imaging determines presence of ischemic stroke and absence of cerebral hemorrhage.

28. The method of any one of claims 23-27, wherein eligibility is determined less than hours after onset of ischemic stroke and the thrombolytic agent is administered more than 3 hours after onset of ischemic stroke.

29. The method of any one of claims 23-27, wherein eligibility is determined less than 4.5 hours after onset of ischemic stroke and the thrombolytic agent is administered more than 4.5 hours after onset of ischemic stroke.

30. The method of any one of claims 23-27, wherein eligibility is determined less than hours after onset of ischemic stroke and the thrombolytic agent is administered more than 4.5 hours after onset of ischemic stroke.

31. The method of any preceding claim, wherein the active agent that inhibits PSD-95 comprises a peptide comprising [E/D/N/Q]-[S/T]-[D/E/Q/N]-[V/L] (SEQ ID NO:1) at the C-terminus or X₁-[T/S]-X₂V (SEQ ID NO:2) at the C-terminus, wherein [T/S] are alternative amino acids, X₁ is selected from among E, Q, and A, or an analogue thereof, X₂ is selected from among A, Q, D, N, N-Me-A, N-Me-Q, N-Me-D, and N-Me-N or an analog thereof, and an internalized peptide linked to the N-terminus of the peptide.

32. The method of claim 31, wherein the active agent that inhibits PSD-95 is nerinetide.

33. The method of any preceding claim, wherein the thrombolytic agent is tPA.

34. A method of treating a subject who has had a stroke with an active agent that inhibits PSD-95, cleavable by plasmin, whereby the active agent is:
administered at least 10 minutes before a thrombolytic agent, or
administered at least 2, 3, 4 or more hours after administration of a thrombolytic agent, or
administered without a thrombolytic agent.

35. The method of claim 34, wherein the active agent that inhibits PSD-95 is administered over a ten minute period and the thrombolytic agent is administered at least 20 minutes from the start of administering the active agent.

36. The method of claim 34, wherein the active agent is a peptide of all L-amino acids.

37. The method of claim 35, wherein the active agent is nerinetide.

38. A method of minimizing degradation of an active agent that inhibits PSD-95, cleavable by plasmin, by a thrombolytic agent, comprising:
administering the active agent that inhibits PSD-95 at least 10 minutes before the thrombolytic agent, or
administering the active agent that inhibits PSD-95 at least 2, 3, 4 or more hours after administration of the thrombolytic agent, or
administering the active agent that inhibits PSD-95 without the thrombolytic agent, or
administering the active agent that inhibits PSD-95 by intranasal or intrathecal administration.

39. The method of claim 38, wherein the active agent that inhibits PSD-95 is administered over a ten minute period and the thrombolytic agent is administered at least 20 minutes from the start of administering the active agent.

40. The method of claim 38, wherein the active agent is a peptide of all L-amino acids.

41. The method of claim 38, wherein the active agent is nerinetide.

42. A method of treating ischemic stroke, comprising administering to a subject having ischemic stroke an active agent that inhibits PSD-95, cleavable by plasmin, and 20-40 minutes after initiating administration of the active agent administering a thrombolytic agent.

43. The method of claim 42, wherein the active agent that inhibits PSD-95 is inhibited over a period of ten minutes and the thrombolytic agent is administered 20-30 minutes after initiating administration of the active agent.
